(19) United States
(12) Patent Application Publication
(10) Pub. No.: US 2024/0252639 A1
(43) Pub. Date: Aug. 1, 2024

(54) COMPOSITIONS AND METHODS FOR PRODUCING AND USING CELL-BASED IMMUNOTHERAPIES TO TARGET TUMORS

(71) Applicant: THE REGENTS OF THE UNIVERSITY OF COLORADO, A BODY CORPORATE, Denver, CO (US)

(72) Inventors: Michael VERNERIS, Denver, CO (US); Jessica LAKE, Denver, CO (US)

(21) Appl. No.: 18/562,035

(22) PCT Filed: Apr. 14, 2022
(86) PCT No.: PCT/US2022/024917

§ 371 Date: Nov. 17, 2023

Related U.S. Application Data
(60) Provisional application No. 63/278,428, filed on Nov. 11, 2021.
(60) Provisional application No. 63/273,053, filed on Oct. 28, 2021.
(60) Provisional application No. 63/175,457, filed on Apr. 15, 2021.

(51) Int. Cl. A61K 39/00; A61K 35/17; A61P 35/00; C07K 14/715; C07K 16/28; C12N 5/0783; C12N 15/86

(52) U.S. Cl. CPC A61K 39/4631; A61K 35/17; A61K 39/4611; A61K 39/464421; A61K 39/464429; A61P 35/00; C07K 14/7158; C07K 16/2827; C12N 5/0636; C12N 15/86; A61K 2239/13; A61K 2239/46; C07K 2317/622; C12N 2501/2308; C12N 2510/00

(57) ABSTRACT

Embodiments relate to novel compositions and methods for treating solid tumors. Other embodiments relate to generating T cells able to home to and treat solid tumors. In accordance with these embodiments, T-cells expressing at least one C-X-C Motif Chemokine Receptor (CXCR) and chimeric antigen receptors (CARs) able to bind to B7H3 are disclosed. In certain embodiments, the present disclosure provides for polynucleotides and vectors encoding a CAR and a CXCR together or in separate constructs, where the CAR binds to B7H3 in solid tumor cells, other tumors, or malignancies. In other embodiments, methods of making T cells expressing a CAR that binds to B7H3 and expressing one or more CXCR are provided. In some embodiments, methods of preventing, treating, or ameliorating tumors or malignancies in a subject are disclosed including administering a composition of T cells expressing at least one CXCR and CARs that bind to B7H3.

### CROSS-REFERENCE

This application claims the benefit of U.S. Provisional Application No. 63/175,457, filed Apr. 15, 2021, U.S. Provisional Application No. 63/273,053, filed Oct. 28, 2021, and U.S. Provisional Application No. 63/278,428 filed Nov. 11, 2021, which applications are incorporated herein by reference.

### FIELD

Embodiments of the instant disclosure relate to novel compositions and methods for generating and using T cells. In some embodiments, T cells are created expressing at least one C-X-C Motif Chemokine Receptor (CXCR) and chimeric antigen receptors (CARs) that bind to B7H3 (B7 Homolog 3, CD276).

### BACKGROUND

Advances in oncology treatment have occurred for some forms of cancer, however, new therapies for sarcoma and other solid tumors have been limited. The five-year cancer-free survival rate is about 20-30% for patients with metastatic osteosarcoma and other conditions such as rhabdomyosarcoma at diagnosis or recurrent states. Therefore, a need exists for targeted and successful treatments for sarcomas and other solid tumors to improve survival.

### SUMMARY

Embodiments of the instant disclosure relate to novel compositions and methods for generating cell-based immunotherapies. In certain embodiment, compositions and methods include the cell-based immunotherapies of a modified CAR T cell therapy or NK cell therapies directed to treating solid tumors or other tumors or other malignancies capable of being treated by cell-based immunotherapies disclosed herein. In other embodiments, the present disclosure provides a single polynucleotide encoding a chimeric antigen receptor (CAR) and a C-X-C chemokine receptor. In some embodiments, polynucleotides can encode a CAR that can be a single-chain variable fragment (scFv) able to bind to or associate with B7H3. In other embodiments, compositions and methods disclosed herein include a CAR T cell capable of binding to B7H3 further transfected with a polynucleotide able to express one or more C-X-C chemokine receptor. In certain embodiments, inclusion of a transfected C-X-C chemokine receptor on CAR T cells disclosed herein improve homing to a solid tumor to improve therapeutic outcome of such a treatment. In other embodiments, inclusion of a transfected C-X-C chemokine receptor on CAR T or other T cells disclosed herein enhance T cell function and alters cell metabolism making the T cells or NK cells more suitable to function in the tumor microenvironment.

In other embodiments, the present disclosure provides cells having at least one single polynucleotide encoding a chimeric antigen receptor (CAR) and a chemokine receptor. In some embodiments, a chemokine receptor disclosed herein can include at least one polynucleotide sequence capable of expressing one or more of C-X-C chemokine receptor 1 (CXCR1), C-X-C chemokine receptor 2 (CXCR2), C-X-C chemokine receptor 3 (CXCR3), C-X-C chemokine receptor 4 (CXCR4), C-X-C chemokine receptor 5 (CXCR5), C-X-C chemokine receptor 6 (CXCR6), C-C chemokine receptor 1 (CCR1), C-C chemokine receptor 2 (CCR2), C-C chemokine receptor 3 (CCR3), C-C chemokine receptor 4 (CCR4), C-C chemokine receptor 5 (CCR5), C-C chemokine receptor 6 (CCR6), C-C chemokine receptor 7 (CCR7), C-C chemokine receptor 8 (CCR8), C-C chemokine receptor 9 (CCR9), C-C chemokine receptor 10 (CCR10), C-C chemokine receptor 11 (CCR11), C chemokine receptor (XCR1), CX₃C chemokine receptor (CX3CR1), or a combination thereof or biologically active fragment thereof.

In certain embodiments, polynucleotides of use herein to express one or more chemokine receptor in a cell encode one or more C-X-C chemokine receptor having a polynucleotide sequence encoding a C-X-C chemokine receptor 1 (CXCR1), a C-X-C chemokine receptor 2 (CXCR2), or a combination thereof or biologically active fragment thereof. In other embodiments, polynucleotides can encode one or more CC (chemokine) receptors (e.g., CX3C or other C receptors). In some embodiments, polynucleotides of use herein to express one or more chemokine receptor in a cell (e.g., CAR T cell) can include a polynucleotide expressing CXCR1 or biologically active fragment thereof. In other embodiments, polynucleotides of use herein to express one or more chemokine receptor in a cell (e.g., CAR T cell) can include a polynucleotide expressing CXCR2 or biologically active fragment thereof. In certain embodiments, polynucleotides for transfection and expression in a cell contemplated herein can encode a CXCR2 having at least 75%, at least 80%, at least 85%, or at least 90%, or at least 95% or up to 100% identity in sequence to a nucleic acid sequence represented by SEQ ID NO: 2 (CXCR2) or biologically active fragment thereof. In some embodiments, polynucleotides for transfection and expression in a cell contemplated herein can encode a CAR having at least 75%, at least 80%, at least 85%, or at least 90%, or at least 95% or up to 100% identity in sequence to a polynucleotide represented by SEQ ID NO: 1 (B7H3 CAR) or biologically active fragment thereof.

In certain embodiments, constructs for delivering any of the polynucleotides disclosed herein include a vector. In some embodiments, vector constructs can be used for creating CAR T cells with honing capabilities to solid tumors. In other embodiments, vectors can be viral vectors. In some embodiments, viral vectors can include but are not limited to, retroviral, lentiviral, baboon pseudotyped vectors or adenoviral vectors or other applicable viral or other vector. In some embodiments, constructs can be delivered to a cell of use herein for example, by electroporation, Crispr and transposon, transpose or other technology known in the art.

In other embodiments, the present disclosure provides cells having or expressing any of the polynucleotides and/or vectors disclosed herein. In some embodiments, cells herein can further include proteins (e.g., receptors) encoded by any of the polynucleotides and/or vector disclosed herein. In some embodiments, cells of use for creating a targeted or specialized CAR T with solid tumor honing properties can include several different types of T cells. In accordance with these embodiments, T cells of use herein can be a CD8+ T cell, a CD4+ T cell, CD8+ and a CD4+ T cell, NK cell, or any combination thereof. In some embodiments, T cells contemplated herein can express αβ TCRs. In other embodiments, T-cells contemplated herein can express γδ TCRs, where these cells have varying phenotype, function, and homing characteristics. In other embodiments, cells disclosed herein can be naive CD8+ T cells, central memory CD8+ T cells, effector memory CD8+ T cell, a bulk CD8+ T cell, or any combination thereof. In some embodiments, cells herein can be a naive CD4+ T cell, a central memory CD4+ T cell, an effector memory CD4+ T cell, a bulk CD4+ T cell, T-cell memory, effector memory RA, NK cells, any T cells, or any combination thereof. In some embodiments, cells herein can be a precursor T cell. In some embodiments, cells herein can be a hematopoietic stem cell.

In some embodiments, pharmaceutical compositions are contemplated which can include any one or more agents including, but not limited to, polynucleotides, polypeptides, vectors, and/or T cells including or housing constructs disclosed herein and at least one pharmaceutically acceptable excipient or carrier as appropriate for the agent of interest. In certain embodiments, methods of administering a composition such as a pharmaceutical composition to a subject having a solid tumor are disclosed.

In other embodiments, methods for preparing cells (e.g., CAR T cells) are described herein. In some embodiments, methods for preparing cells can include any or some of the following procedures such as: (i) introducing a vector disclosed herein into a lymphocyte cell; (ii) culturing the T lymphocyte cell containing the vector in media to expand the culture and (iii) enriching for vector-containing T lymphocyte cells and (iv) adding cytokines or chemokines (e.g. CXCL8 or others) to the culture media to enhance expansion of the T cell populations. In some embodiments, the modified T lymphocytes can be harvested and stored for later use. In other embodiments, the T lymphocytes can be harvested and prepared for use in a subject in need thereof. In some embodiments, media for culturing the T lymphocyte cells can include, but is not limited to, anti-CD3 antibody, an anti-CD28 antibody, cytokines (e.g., cytokines known in the art in induce expansion of T lymphocytes), or any combination thereof. In some embodiments, cytokines of use here can include, but are not limited to, IL-15, IL-7, IL-2, IL-12, IL-18, IL-21, or a combination thereof. In some embodiments, enriching the vector-containing T lymphocyte population can include contacting the T lymphocyte cells with a selection reagent. In certain embodiments, a selection reagent can be methotrexate or other suitable selection reagent. In some embodiments, a T lymphocyte for use in the methods herein can be a CD8+ T lymphocyte, a CD4+ T lymphocyte, precursor T cell or a mixed population thereof. In other embodiments, a media disclosed herein can include, but is not limited to, L-glutamine, streptomycin sulfate, gentamicin sulfate and other agents. In other embodiments, media can include fetal bovine serum or can be serum-free media, HEPES buffer, and Glutamax.

In certain embodiments, methods of treating, reducing growth or expansion, or ameliorating, reducing the tumor size, or inducing apoptosis of solid tumors and solid tumor cells in a subject are disclosed. In some embodiments, methods of treating reducing growth or expansion, or ameliorating, reducing the tumor size, or inducing apoptosis of solid tumors and solid tumor cells can include administering an effective number of modified T-cells having any of the polynucleotide vectors, or expressing the disclosed polypeptides contemplated herein, or pharmaceutical compositions thereof to a subject having a tumor.

In some embodiments, these methods can include combination treatments. In other embodiments, a combination treatment can include irradiating the tumor prior to, simultaneously, or after administering an effective amount of polynucleotides, polypeptides, vectors expressing polypeptides disclosed herein, cells expressing polypeptides disclosed herein, or pharmaceutical compositions disclosed herein to the subject. In other embodiments, methods for treating the subject can include treating a subject having or suspected of developing a tumor can include irradiating the tumor before, during or after administering treatments disclosed herein. In accordance with these embodiments, radiation dosage can range from about 1 Gy to about 100 Gy, or about 2 Gy to about 60 Gy or other suitable dose of radiation one time or more to the subject. Radiation can be administered to the subject prior to, simultaneously, or after administering an effective concentration of polynucleotides, polypeptides, vectors expressing polypeptides disclosed herein, cells expressing polypeptides disclosed herein, or pharmaceutical compositions disclosed herein to the subject. In some embodiments, administration of the polynucleotides, polypeptides, vectors expressing polypeptides disclosed herein, cells expressing polypeptides disclosed herein, or pharmaceutical compositions disclosed herein to a subject can be by intravenous, bolus, by catheter or other method known in the art. In some embodiments, administration of the polynucleotides, polypeptides, vectors containing constructs disclosed herein, cells, or pharmaceutical compositions disclosed herein to a subject can be systemic, localized or directly applied to a tumor site or introduced to the tumor(s) in the subject.

In some embodiments, tumors in a subject can be targeted treated by the methods herein that can include but is not limited to, exposing the tumors to cells expressing at least one of B7H3⁺, IL-8+ or a combination thereof. In accordance with these embodiments, tumors treated by the methods disclosed herein can be a solid tumor or soft tumor, or other tumor or malignancies. In accordance with these embodiments, a solid tumor can include, but is not limited to, breast, lung, brain, head and neck, prostate, stomach or other gastrointestinal tumor, colon, liver, kidney, eye, skin, or other solid tumor or leukemia (e.g., acute myeloid leukemia (AML)). In other embodiments, a solid tumor can be a sarcoma.

In other embodiments, tumors treated by the methods disclosed herein can include, but are not limited to, a bone sarcoma, a soft-tissue sarcoma, or a combination thereof. In other embodiments, adenocarcinomas are contemplated. In yet other embodiments, GI tract carcinomas are contemplated for treatment herein. In other embodiments, tumors treated by compositions and methods disclosed herein can be, but are not limited to, osteosarcoma, chondrosarcoma, poorly differentiated round/spindle cell tumors, Ewing sarcoma, hemangioendothelioma, angiosarcoma, fibrosarcoma/myofibrosarcoma, chordoma, adamantinoma, liposarcoma, leiomyosarcoma, malignant peripheral nerve sheath tumor, rhabdomyosarcoma, synovial sarcoma, malignant solitary fibrous tumor, or any combination thereof. In some embodiments, tumors treated by compositions and methods disclosed herein can be, but are not limited to, liposarcoma, atypical lipomatous tumor, dermatofibrosarcoma protuberans, malignant solitary fibrous tumor, inflammatory myofibroblastic tumor, low-grade myofibroblastic sarcoma, fibrosarcoma, myxofibrosarcoma, low-grade fibromyxoid sarcoma, giant cell tumor of soft tissues, leiomyosarcoma, malignant glomus tumor, rhabdomyosarcoma, hemangioendothelioma, angiosarcoma of soft tissue, extraskeletal osteosarcoma, gastrointestinal stromal tumor, malignant, malignant peripheral nerve sheath tumor, malignant Triton tumor, malignant granular cell tumor, malignant ossifying fibromyxoid tumor, stromal sarcoma not otherwise specified, myoepithelial carcinoma, malignant phosphaturic mesenchymal tumor, synovial sarcoma, epithelioid sarcoma, alveolar soft part sarcoma, clear cell sarcoma of soft tissue, extraskeletal myxoid chondrosarcoma, extraskeletal Ewing sarcoma, desmoplastic small round cell tumor, extrarenal rhabdoid tumor, perivascular epithelioid cell tumor, intimal sarcoma, undifferentiated spindle cell sarcoma, undifferentiated pleomorphic sarcoma, undifferentiated round cell sarcoma, undifferentiated epithelioid sarcoma, undifferentiated sarcoma, not otherwise specified, or any combination thereof.

In certain embodiments, kits are provided for storage, transport and use in treating, eliminating, or reducing the size of, or reducing expansion of, one or more tumor in a subject. In some embodiments, compositions containing cells expressing a construct disclosed herein can be stored in a container, frozen for later use or transported for immediate use. In some embodiments, a kit can contain at least one container of cells disclosed herein and stored for short or prolonged periods at an appropriate temperature. In certain embodiments, the present disclosure provides kits for preparing T cells disclosed herein for use in in compositions and methods.

### BRIEF DESCRIPTION OF THE DRAWINGS

The following drawings form part of the present specification and are included to further demonstrate certain embodiments of the present disclosure. Certain embodiments can be better understood by reference to one or more of these drawings in combination with the detailed description of specific embodiments presented herein.

FIGS. 1A-IC represent an exemplary experiment illustrating IL-8 expression in human sarcoma cells without radiation (FIG. 1A) and after irradiation (FIG. 1B), where FIG. 1C represents a bar graph illustrating various expressions of IL-8 in difference solid tumor cell lines in accordance with certain embodiments of the present disclosure.

FIG. 2 represents an exemplary experiment illustrating a lack of IL-8 expression in an IL-8 knockout rhabdomyosarcoma (RMS) cell line in accordance with certain embodiments of the present disclosure.

FIG. 3 represents an exemplary experiment illustrating IL-8 gene expression in osteosarcoma tumors on day 3 and day 7 after tumor irradiation is demonstrated in accordance with certain embodiments of the present disclosure.

FIG. 4 represents an exemplary experiment illustrating B7H3 (CD276) expression in human rhabdomyosarcoma (RMS) cell lines in accordance with certain embodiments of the present disclosure.

FIG. 5 represents an exemplary experiment illustrating B7H3 (CD276) expression in a human osteosarcoma (OS) cell line in accordance with certain embodiments of the present disclosure.

FIG. 6 represents an exemplary experiment illustrating a vector map of an exemplary construct of a polynucleotide plasmid including B7H3 and an IL-8 receptor (CXCR2) encoding regions in accordance with certain embodiments of the present disclosure.

FIGS. 7A-7F represent an exemplary experiment illustrating flow cytometry of T cells gated for lymphocytes (FIG. 7A), single cells (FIG. 7B), live cells (FIG. 7C), cells expressing CD56-CD3+ (FIG. 7D), B7H3 CAR+ T cells (FIG. 7E), and CXCR2+B7H3 CAR+ T cells (FIG. 7F) in accordance with certain embodiments of the present disclosure.

FIGS. 8A-8G represent an exemplary experiment illustrating flow cytometry of T cells that were non-transduced controls, transduced with B7H3 CAR, or transduced with CXCR2-T2A-B7H3 CAR in accordance with certain embodiments of the present disclosure.

FIG. 9 represents an exemplary experiment illustrating representing transwell migration toward an IL-8 gradient by various cells including neutrophils, non-transduced T cell controls, B7H3 CAR-expressing T cells, and B7H3 CAR-T2A-CXCR2 CAR-expressing T cells in accordance with certain embodiments of the present disclosure.

FIG. 10 represents an exemplary experiment illustrating cell killing of human sarcoma cells by non-transduced T cell controls, B7H3 CAR-expressing T cells, and B7H3 CAR-T2A-CXCR2 CAR-expressing T cells in the presence or absence of irradiation in accordance with certain embodiments of the present disclosure.

FIG. 11 represents an exemplary experiment illustrating migration of B7H3 CAR T cells and B7H3-CXCR2 CAR T cells using a live-cell imaging chemotaxis assay in accordance with certain embodiments of the present disclosure.

FIG. 12 represents an exemplary experiment illustrating interferon-γ (INF-γ) and interleukin-2 (IL-2) levels in B7H3-CXCR2 CAR T cells and B7H3 CAR T cells after co-culturing with irradiated rhabdomyosarcoma in accordance with certain embodiments of the present disclosure.

FIG. 13 represents an exemplary experiment illustrating caspase-3 activity in tumor spheroids cultured alone, co-cultured with B7H3 CAR T cells, or co-cultured with B7H3-CXCR2 CAR T cells for 2 hours and 48 hours in accordance with certain embodiments of the present disclosure.

FIG. 14 represents an exemplary experiment illustrating the kinetics of caspase-3 activity (killing) over time in tumor spheroids when co-cultured with B7H3 CAR T cells or B7H3-CXCR2 CAR T cells in accordance with certain embodiments of the present disclosure.

FIG. 15 represents an exemplary experiment illustrating a Western blot analysis of phosphorylated AKT and total AKT in B7H3 CAR T cells and B7H3-CXCR2 CAR T cells with and without IL-8 stimulation in accordance with certain embodiments of the present disclosure.

FIG. 16 represents an exemplary experiment illustrating bioluminescent imaging of mice injected with IL-8 overexpressing rhabdomyosarcoma tumor cells prior to CAR T cell infusion (Day 0) and after CAR T cell infusion and a Kaplan Meier (survival) curve representative of mice survival in accordance with certain embodiments of the present disclosure.

FIG. 17 represents an exemplary experiment illustrating mitochondrial respiration and glycolytic activity in unstimulated or IL-8 stimulated T cells, B7H3 CAR T cells and B7H3-CXCR2 CAR T cells in accordance with certain embodiments of the present disclosure.

FIG. 18 represents an exemplary experiment illustrating increased metabolic potential of B7H3-CXCR2 CAR T cells compared to B7H3 CAR T cells. A and B) T-cell bioenergetic phenotype at basal and in response to IL-8 (50 ng/ml) was assessed using a seahorse mitochondrial stress test (n=3). Open square: Basal OCR vs ECAR. Closed square Stressed OCR vs ECAR. Dotted line indicates metabolic potential. (C) ATP production from stress test, resting values depicted in squares and IL-8 stimulated values in circles. (D) Basal mitochondrial mass as determined by mitotracker staining. Representative of n=4.

FIG. 19 represents an exemplary experiment illustrating mitochondrial stress in B7H3 CAR T cells (white) compared to B7H3-CXCR2 CAR T cells (gray). A-B), CAR T cell OXPHOS at basal (squares) and in response to IL-8 (50 ng/ml, circles), C) RH30 and RH30-IL-8 co-culture for 48 hrs was assessed by a seahorse mitochondrial stress test. Squares: No IL-8 Circles: +IL-8 (n=3). D) mean fluorescent intensity (MFI) of CD36 on B7H3 CAR T vs B7H3-CXCR2 CAR T (n=4).

FIG. 20 represents an exemplary experiment illustrating flow cytometry of T cells gated for lymphocytes (FIG. 20A), single cells (FIG. 20B), and live cells (FIG. 20C).

FIG. 21 represents an exemplary experiment illustrating flow cytometry of non-transduced T cells demonstrating no expression of both B7H3 (FIG. 21A) and CXCR1 (FIG. 21B).

FIG. 22 represents an exemplary experiment illustrating flow cytometry of T cells demonstrating expression of B7H3 (FIG. 22A) and CXCR1 (FIG. 22B).

FIG. 23 represents an exemplary experiment illustrating flow cytometry of T cells demonstrating expression of B7H3 (FIG. 23A) and CXCR2 (FIG. 23B).

FIG. 24 represents an exemplary experiment illustrating flow cytometry of T cells demonstrating expression of B7H3 (FIG. 24A), CXCR1, and CXCR2 (FIG. 24B).

FIG. 25 represents an exemplary experiment illustrating flow cytometry of T cells demonstrating expression of B7H3 (FIG. 25A) and CXCR1 (FIG. 25B).

FIG. 26 represents an exemplary experiment illustrating flow cytometry of T cells demonstrating expression of B7H3 (FIG. 26A) and CXCR2 (FIG. 26B).

FIG. 27 represents an exemplary experiment illustrating flow cytometry of T cells demonstrating expression of B7H3 (FIG. 27A), CXCR1, and CXCR2 (FIG. 27B).

FIG. 28 represents an exemplary experiment illustrating migration of B7H3 CAR T cells and B7H3-CXCR2 CAR T cells using a live-cell imaging chemotaxis assay in accordance with certain embodiments of the present disclosure.

### DEFINITIONS

Terms, unless defined herein, have meanings as commonly understood by a person of ordinary skill in the art relevant to certain embodiments disclosed herein or as applicable.

As used herein “about” unless otherwise indicated, applies to all numbers expressing quantities of agents and/or compounds, properties such as molecular weights, reaction conditions, and as disclosed herein are contemplated as being modified in all instances by this term. Accordingly, unless indicated to the contrary, the numerical parameters in the specification and claims are approximations that can vary from about 10% to about 15% plus and/or minus depending upon the desired properties sought as disclosed herein. Numerical values as represented herein inherently contain standard deviations that necessarily result from the errors found in the numerical value's testing measurements.

As used herein, “individual”, “subject”, “host”, and “patient” can be used interchangeably herein and refer to any mammalian subject for whom diagnosis, treatment, prophylaxis, or therapy is desired, for example, humans, pets, livestock, horses, or other animals.

As used herein, “treat,” “treating” or “treatment” can refer to reversing, ameliorating, or inhibiting onset or inhibiting progression of a health condition or disease or a symptom of the health condition or disease.

As used herein, “polynucleotide,” “nucleic acid” or “nucleic acid molecule” or “nucleic acid sequence” include deoxyribonucleic acid (DNA) or ribonucleic acid (RNA), mRNA, oligonucleotides, and the like.

As used herein, “vector”, “expression vector” or “construct” refers to nucleic acid components used to introduce polynucleotides into a cell having regulatory elements to provide expression of the heterologous nucleic acids in the cell; for example, to express an encoded polypeptide. Vectors include but are not limited to plasmid, minicircles, yeast, and/or viral genomes. In some alternatives, the vectors are plasmid, minicircles, or viral genomes. In some alternatives, the vector is a viral vector. In some embodiments, the viral vector is a lentivirus. In some alternatives, the vector is a lentiviral vector. In some embodiments, the vector is a foamy viral vector, adenoviral vectors, retroviral vectors, or lentiviral vectors. In some embodiments, the construct comprises a donor template, wherein the donor template comprises homology arms for recombination using any suitable genomic editing technology, for example CRISPR-cas.

As used herein, “chimeric antigen receptor” or “CAR” or “chimeric T cell receptor” refers herein to a synthetically designed receptor having a ligand binding domain of an antibody or another peptide sequence that binds to a molecule associated with the disease or disorder and is linked via a spacer domain to one or more intracellular signaling domains of a T cell or other receptors, such as a costimulatory domain. Chimeric receptor can also be referred to as artificial T cell receptors, chimeric T cell receptors, chimeric immunoreceptors, and chimeric antigen receptors (CARs).

### DETAILED DESCRIPTION

In the following sections, certain exemplary compositions and methods are described in order to detail certain embodiments of the invention. It will be obvious to one skilled in the art that practicing the certain embodiments does not require the employment of all or even some of the specific details outlined herein, but rather that concentrations, times, and other specific details can be modified through routine experimentation. In some cases, well known methods, or components have not been included in the description.

In some embodiments, compositions and methods disclosed herein are designed to treat cancer, such as solid tumors or other tumors disclosed herein or other malignancies. Prior to this disclosure, cell-based immunotherapies using chimeric antigen receptor (CAR) T cells have had benefits in leukemia and other blood-related cancers but have been unsuccessful in treating solid tumors. Further, it is known that in most instances, CAR T cell therapies have been unsuccessful at treating solid tumors such as sarcomas. One of the major limitations has been the inability of CAR T cells to find, home to or localize to a solid tumor once administered to a subject. As such, there is a need in the art for improved cell-based immunotherapies using CAR T cells having improved cell homing to solid tumors to treat a subject having a solid tumor.

Certain embodiments of the present disclosure relate to novel constructs, compositions, and methods for generating and using polynucleotides encoding one or more chimeric antigen receptors (CAR) or biologically active fragments thereof and at least one chemokine receptors, such as a C-C Motif and/or C-X-C Motif Chemokine Receptor, or biologically active fragment thereof for targeting solid tumors. In accordance with these embodiments, Interleukin 8 ((IL-8) or chemokine (C-X-C motif) ligand 8, CXCL8)) is a chemokine that binds to its cognate receptors, C-X-C chemokine receptor 1 (CXCR1) and C-X-C chemokine receptor 2 (CXCR2).

The polynucleotide constructs can encode any suitable number of CARs, such as at least 1, 2, 3, 4, 5, 6, 7, 8, or 9 and/or no more than 10, 9, 8, 7, 6, 5, 4, 3, or 2 CARs, for example 1-10, preferably 1-8, more preferably 1-6, even more preferably 1-4, still more preferably 1-2. In certain embodiments, the polynucleotide construct encodes one CAR. In certain embodiments, the CAR is a B7H3 CAR. In certain embodiments, the polynucleotide construct encodes a B7H3 CAR and at least 1, 2, 3, 4, 5, 6, 7, or 8 and/or no more than 9, 8, 7, 6, 5, 4, 3, or 2 additional CARs. In preferred embodiments, the polynucleotide construct encodes a B7H3 CAR and 1-7 additional CARs, more preferred embodiments 1-5 additional CARs, even more preferred embodiments, 1-3 additional CARs, still more preferred embodiments 1 additional CAR.

The polynucleotide constructs can encode any suitable number of chemokine receptors, such as at least 1, 2, 3, 4, 5, 6, 7, 8, or 9 and/or no more than 10, 9, 8, 7, 6, 5, 4, 3, or 2 chemokine receptors, for example 1-10, preferably 1-8, more preferably 1-6, even more preferably 1-4, still more preferably 1-2. In certain embodiments, the polynucleotide constructs encode one or more CARs and/or one or more chemokine receptors, such as a CCR and/or CXCR, for example CXCR1 or CXCR2.

In certain embodiments, the polynucleotide constructs encode a single CAR and at least 1, 2, 3, 4, 5, 6, 7, 8, or 9 and/or no more than 10, 9, 8, 7, 6, 5, 4, 3, or 2 chemokine receptors, for example 1-10, preferably 1-8, more preferably 1-6, even more preferably 1-4, still more preferably 1-2. In certain embodiments, the CAR is a B7H3 CAR. In certain embodiments, the polynucleotide construct encodes a single B7H3 CAR and CXCR1 and/or CXCR2; preferably a B7H3 CAR and CXCR1; more preferably a B7H3 CAR and CXCR2; even more preferably a B7H3 CAR, CXCR1, and CXCR2.

In certain embodiments, the polynucleotide constructs encode two or more CARs and at least 1, 2, 3, 4, 5, 6, 7, 8, or 9 and/or no more than 10, 9, 8, 7, 6, 5, 4, 3, or 2 chemokine receptors, for example 1-10, preferably 1-8, more preferably 1-6, even more preferably 1-4, still more preferably 1-2. In certain embodiments, the polynucleotide construct encodes a first CAR and a second CAR, wherein the first CAR is a B7H3 CAR and the second CAR is any suitable CAR for the intended application. In certain embodiments, the polynucleotide construct encodes a first B7H3 CAR, a second CAR, and CXCR1 and/or CXCR2; preferably a first B7H3 CAR, a second CAR, and CXCR1; more preferably a first B7H3 CAR, a second CAR, and CXCR2; even more preferably a first B7H3 CAR, a second CAR, CXCR1, and CXCR2.

The polynucleotide constructs can comprise a single polynucleotide, wherein the single polynucleotide comprises a sequence for each encoded component, for example a CAR and a CXCR. Additionally or alternatively, the polynucleotide constructs can comprise one or more polynucleotides, wherein the plurality of polynucleotides together comprise the sequences for each encoded component, for example a first polynucleotide encoding for a CAR, e.g., a CAR polynucleotide, and a second polynucleotide encoding for a CXCR, e.g., a CXCR polynucleotide.

In certain embodiments, the polynucleotide constructs are delivered to a cell, at least a portion of the polynucleotide constructs are optionally integrated into a genome of the cell using any suitable technique, wherein the one or more polypeptides encoded by the polynucleotide constructs are expressed on a surface of the cell, whereby the cell demonstrates utility for targeting solid tumors. In accordance with these embodiments, any of the polynucleotide constructs disclosed herein can be used to express the corresponding polypeptides in a suitable transfected cell, such as a lymphocyte cell (e.g., T cell, NK cell, or other lymphocyte cell, or precursor cell) and/or a stem cell (e.g., hematopoietic stem cell, iPSC, or other stem cell).

In certain embodiments, provided herein are polynucleotide constructs encoding a CAR or biologically active fragment thereof and one or more chemokine receptors, such as CCR and/or CXCR, or a biologically active fragment thereof. In certain embodiments, the polynucleotide construct comprises a first polynucleotide encoding a CAR or biologically active fragment thereof and a second polynucleotide encoding one or more chemokine receptors or a biologically active fragment thereof. In certain embodiments, the polynucleotide constructs disclosed herein comprise a first polynucleotide encoding a CAR and second polynucleotide encoding CXCR1 or biologically active fragment thereof. In certain embodiments, polynucleotide constructs disclosed herein comprise a first polynucleotide encoding a CAR and second polynucleotide encoding CXCR2 or biologically active fragment thereof. In certain embodiments, the first and second polynucleotides are separate polynucleotides. In preferred embodiments, the first and second polynucleotides are the same polynucleotide.

The polynucleotide constructs can comprise any suitable number of polynucleotides encoding chemokine receptors depending on the application, wherein each additional chemokine receptor is different from the prior chemokine receptors. In other embodiments, compositions disclosed herein comprise a first polynucleotide encoding a CAR, a second polynucleotide encoding CXCR1, and a third polynucleotide encoding CXCR2 or biologically active fragment thereof. Without being bound by theory, it is contemplated that CXCR1 and CXCR2 can activate different intracellular signal cascade networks resulting in different cellular responses, and the combination of both CXCR1 and CXCR2 receptor provide enhanced cellular function in an intended microenvironment. In certain embodiments, the first, second, and/or third polynucleotides are separate polynucleotides. In certain embodiments, the first, second, and/or third polynucleotides are the same polynucleotide.

The CAR can comprise a binding domain, e.g., a scFv, capable of binding to or associating with any suitable binding partner, e.g., antigen, such as a B7H3 binding partner. In certain embodiments, the binding domain comprises a polypeptide that binds to B7H3, e.g., a B7H3 CAR. In certain embodiments, the B7H3 CAR can be encoded by a polynucleotide having at least 75%, at least 80%, at least 85% identity (e.g., at least about 85%, at least about 90%, at least about 95%, at least about 99%, up to 100% identity) to a polynucleotide represented by SEQ ID NO: 1, as inserted below. In certain embodiments, the B7H3 CAR can be encoded by a polynucleotide comprising a sequence at least 75, 80, 85, 90, 95, 96, 97, 98, 99, or 100% identical to SEQ ID NO: 1, preferably 80-100% identical, more preferably 90-100% identical, more preferably 95-100% identical.

The chemokine receptor can comprise any suitable receptor, such as a CCR or a CXCR, such as a C-X-C chemokine receptor 1 (CXCR1), C-X-C chemokine receptor 2 (CXCR2), C-X-C chemokine receptor 3 (CXCR3), C-X-C chemokine receptor 4 (CXCR4), C-X-C chemokine receptor 5 (CXCR5), C-X-C chemokine receptor 6 (CXCR6), C-C chemokine receptor 1 (CCR1), C-C chemokine receptor 2 (CCR2), C-C chemokine receptor 3 (CCR3), C-C chemokine receptor 4 (CCR4), C-C chemokine receptor 5 (CCR5), C-C chemokine receptor 6 (CCR6), C-C chemokine receptor 7 (CCR7), C-C chemokine receptor 8 (CCR8), C-C chemokine receptor 9 (CCR9), C-C chemokine receptor 10 (CCR10), C-C chemokine receptor 11 (CCR11), C chemokine receptor (XCR1), CX₃C chemokine receptor (CX3CR1), or a combination thereof or biologically active fragment thereof, preferably CXCR1 and/or CXCR2. In preferred embodiments, the chemokine receptor is capable of binding to or associated with Interleukin 8 (IL-8). In certain embodiments, CXCR2 is encoded by a polynucleotide that shares at least 75%, at least 80%, at least 85% identity (e.g., at least about 85%, at least about 90%, at least about 95%, at least about 99%, up to 100% identity) to a polynucleotide represented by SEQ ID NO: 2, as provided below. In certain embodiments, CXCR2 is encoded by a polynucleotide comprising a sequence at least 75, 80, 85, 90, 95, 96, 97, 98, 99, or 100% identical to SEQ ID NO: 2, preferably 80-100% identical, more preferably 90-100% identical, more preferably 95-100% identical. In certain embodiments, CXCR1 is encoded by a polynucleotide that shares at least 75%, at least 80%, at least 85% identity (e.g., at least about 85%, at least about 90%, at least about 95%, at least about 99%, up to 100% identity) to a polynucleotide represented by SEQ ID NO: 11, as provided below. In certain embodiments, CXCR1 is encoded by a polynucleotide comprising a sequence at least 75, 80, 85, 90, 95, 96, 97, 98, 99, or 100% identical to SEQ ID NO: 11, preferably 80-100% identical, more preferably 90-100% identical, more preferably 95-100% identical.

In certain embodiments, provided herein are polynucleotide constructs comprising a first polynucleotide encoding a CAR and a second polynucleotide encoding a CXCR2 wherein the CXCR2 the second polynucleotide shares at least 75%, at least 80%, at least 85% identity (e.g., at least about 85%, at least about 90%, at least about 95%, at least about 99%, up to 100% identity) to a polynucleotide represented by SEQ ID NO: 2, as provided below. In certain embodiments, the second polynucleotide comprises a sequence at least 75, 80, 85, 90, 95, 96, 97, 98, 99, or 100% identical to SEQ ID NO: 2, preferably 80-100% identical, more preferably 90-100% identical, more preferably 95-100% identical. In certain embodiments, the first and the second polynucleotides are the same polynucleotide. As used herein, the phrase “shares X % identity with” is synonymous with and can be used interchangeably with the phrase “is X % identical to”.

(CXCR2)
SEQ ID NO: 2
ATGGAAGATTTTAACATGGAGAGTGACAGCTTTGAAGATTTCTGG
AAAGGTGAAGATCTTAGTAATTACAGTTACAGCTCTACCCTGCCC
CCTTTTCTACTAGATGCCGCCCCATGTGAACCAGAATCCCTGGAA
ATCAACAAGTATTTTGTGGTCATTATCTATGCCCTGGTATTCCTG
CTGAGCCTGCTGGGAAACTCCCTCGTGATGCTGGTCATCTTATAC
AGCAGGGTCGGCCGCTCCGTCACTGATGTCTACCTGCTGAACCTA
GCCTTGGCCGACCTACTCTTTGCCCTGACCTTGCCCATCTGGGCC
GCCTCCAAGGTGAATGGCTGGATTTTTGGCACATTCCTGTGCAAG
GTGGTCTCACTCCTGAAGGAAGTCAACTTCTATAGTGGCATCCTG
CTACTGGCCTGCATCAGTGTGGACCGTTACCTGGCCATTGTCCAT
GCCACACGCACACTGACCCAGAAGCGCTACTTGGTCAAATTCATA
TGTCTCAGCATCTGGGGTCTGTCCTTGCTCCTGGCCCTGCCTGTC
TTACTTTTCCGAAGGACCGTCTACTCATCCAATGTTAGCCCAGCC
TGCTATGAGGACATGGGCAACAATACAGCAAACTGGCGGATGCTG
TTACGGATCCTGCCCCAGTCCTTTGGCTTCATCGTGCCACTGCTG
ATCATGCTGTTCTGCTACGGATTCACCCTGCGTACGCTGTTTAAG
GCCCACATGGGGCAGAAGCACCGGGCCATGCGGGTCATCTTTGCT
GTCGTCCTCATCTTCCTGCTCTGCTGGCTGCCCTACAACCTGGTC
CTGCTGGCAGACACCCTCATGAGGACCCAGGTGATCCAGGAGACC
TGTGAGCGCCGCAATCACATCGACCGGGCTCTGGATGCCACCGAG
ATTCTGGGCATCCTTCACAGCTGCCTCAACCCCCTCATCTACGCC
TTCATTGGCCAGAAGTTTCGCCATGGACTCCTCAAGATTCTAGCT
ATACATGGCTTGATCAGCAAGGACTCCCTGCCCAAAGACAGCAGG
CCTTCCTTTGTTGGCTCTTCTTCAGGGCACACTTCCACTACTCTC

In certain embodiments, provided herein are polynucleotide constructs comprising a first polynucleotide encoding a CAR and a second polynucleotide encoding at least one chemokine receptor, wherein the CAR specifically binds to a B7 Homolog 3 (B7H3) binding partner or biologically active fragments thereof. In some embodiments, the first polynucleotide encodes a CAR that specifically binds to human B7 Homolog 3 (B7H3) and the second polynucleotide encodes at least one CXCR. In certain embodiments, the second polynucleotide encodes for CXCR1, CXCR2, or both.

In other embodiments, polynucleotides provided herein are polynucleotide constructs comprising a first polynucleotide encoding a CAR that associates with or binds to a B7H3 binding partner or biologically active fragments thereof and a second polynucleotide encoding at least one chemokine receptor, such as CXCR1, CXCR2, or both. In some embodiments, the first polynucleotide encodes a CAR that associates with or binds to B7H3 or biologically active fragments thereof and the second polynucleotide encodes CXCR2. In some embodiments, the first polynucleotide encodes a CAR that associates with or binds to B7H3 or biologically active fragments thereof and the second polynucleotide encodes CXCR1. In certain embodiments, the composition further comprises a third polynucleotide comprising a CXCR different from the first CXCR. For example, the second polynucleotide comprises CXCR1, and the third polynucleotide comprises CXCR2. In some embodiments, polynucleotides constructs disclosed herein can encode a CAR that associates with, or binds to, B7H3 and CXCR2 or biologically active fragment thereof wherein the CXCR2 can be encoded by a polynucleotide having at least 75%, at least 80%, at least 85% identity (e.g., at least about 85%, at least about 90%, at least about 95%, at least about 99%, up to 100% identity) to a polynucleotide represented by SEQ ID NO: 2 or encoded polypeptide thereof. In certain embodiments, polynucleotide constructs for transfection and expression in a cell contemplated herein can encode a CXCR2 having at least 85%, or at least 90%, or at least 95% identity, or at least 99% identity in sequence to a polynucleotide represented by SEQ ID NO: 2 (CXCR2) or biologically active fragment thereof or encoded polypeptide thereof. In other embodiments, polynucleotide constructs for transfection and expression in a cell contemplated herein can encode a CAR having at least 75%, at least 80%, at least 85% identity (e.g., at least about 85%, at least about 90%, at least about 95%, at least about 99%, up to 100% identity) in sequence to a polynucleotide represented by SEQ ID NO: 1 (B7H3 CAR) or biologically active fragment thereof.

In other embodiments, inclusion of a transfected chemokine receptor, such as a CXCR, on CAR T or other T cells disclosed herein enhance T cell function and alters cell metabolism making the T cells or NK cells more suitable to function in the tumor microenvironment. In accordance with these embodiments, lymphocytes transfected or edited to express or over express one or more chemokine receptor are contemplated of use to generate a population of cells to enhance tumor killing of the lymphocytes. In some embodiments, one or more CXCR can include at least one of CXCR1 and/or CXCR2. In certain embodiments, a cell comprising one or more CXCR demonstrate increased mitochondrial mass. In certain embodiments, the cell comprises at least 1.2, 1.4, 1.6, 1.8, 2, 4, 5, 6, 7, 8, or 9-fold and/or no more than 10, 9, 8, 7, 6, 5, 4, 2, 1.8, 1.6, or 1.4-fold increased mitochondrial mass as compared to a corresponding cell lacking a chemokine receptor, for example 1.2 to 10-fold increased mitochondrial mass, preferably at least 2-fold increased mitochondrial mass as compared to a corresponding cell lacking a chemokine receptor. In certain embodiments, a cell comprising one or more CXCR demonstrate increased ATP production. In certain embodiments, the cell comprises at least 1.2, 1.4, 1.6, 1.8, 2, 4, 5, 6, 7, 8, or 9-fold and/or no more than 10, 9, 8, 7, 6, 5, 4, 2, 1.8, 1.6, or 1.4-fold increased ATP production as compared to a corresponding cell lacking a chemokine receptor, for example 1.2 to 10-fold increased ATP production, preferably at least 2-fold increased ATP production as compared to a corresponding cell lacking a chemokine receptor.

In some embodiments, CAR T-cells disclosed herein expressing one or more CXCR can target an antigen expressed on tumor cells such as one or more T-cell antigens including, but not limited to, one or more of CD7, CD2 and CD3. In certain embodiments, a control CAR-T cell (e.g., without expressing constructs disclosed herein) can bind to a CD19 antigen. In some embodiments, a control CAR T-cell expressing only a B7H3 receptor can target B7H3 but does not target solid tumor cells as provided herein. In other embodiments, a CAR T cell that can specifically bind to B7H3 includes an antigen binding domain that specifically binds to a B7H3 antigen. In certain embodiments, an antigen binding domain for use herein can be an antibody, an antigen-binding fragment of an antibody, or a fusion protein derived from such an antibody, such as a single-chain variable fragment (scFv). In accordance with these embodiments herein, a scFv can include a single chain Fv antibody in which the variable domains of the heavy chain and of the light chain of a traditional two chain antibody have been joined to form a single polypeptide chain. In some embodiments, single chain antibodies contemplated herein can be derived from any species including human or animal (e.g., mice, rabbit, pig, dog, cow, horse, goat, camel, or other animal). In some embodiments, the intracellular signaling domain can include a signaling domain and a co-stimulatory domain. In other embodiments, a CAR T cells that can specifically bind to B7H3 as disclosed herein can include a single-chain variable fragment (scFv) that specifically binds to B7H3 on a tumor. In some embodiments, the antigen binding domain of a CAR T cell that specifically binds to B7H3 can include a scFv derived from an antibody which specifically binds to B7H3. It is contemplated herein that CAR T cells disclosed herein can further include one or more of these additional features.

In other embodiments, a polynucleotide construct disclosed herein encoding a B7H3 CAR can include a spacer domain which links the antigen binding domain to a transmembrane domain. In some embodiments, a spacer domain of appropriate length can improve mobility of an antigen binding domain to allow for optimal binding to a target antigen and improve flexibility. In certain embodiments, a spacer domain can be derived from at least a portion or segment of a hinge region of an IgG1, IgG2, IgG3, or IgG4. In some embodiments, a spacer domain can be derived from a CH2 region and/or CH3 region of an IgG1, IgG2, IgG3, or IgG4. In other embodiments, a spacer domain can include upper hinge amino acids found between the variable heavy chain and the core, and the core hinge amino acids including a polyproline region. In other embodiments, a spacer region disclosed herein can be derived from at least a portion of a hinge region of a human IgG4 hinge spacer. In some embodiments, a spacer region for use herein can be a human IgG4 hinge-CH3 spacer. It is contemplated that any of these B7H3 CAR constructs can be used in a single construct in combination with one or more CXCRs (e.g., CXCR1, CXCR2) disclosed herein for use in T cell transfection and use in therapeutic applications.

In some embodiments, a polynucleotide construct disclosed herein encoding a B7H3 CAR and at least one CXCR can include a transmembrane domain. A transmembrane domain can provide anchoring of a CAR in a cell membrane. In some embodiments, a transmembrane domain can be derived from a membrane-bound or transmembrane protein. In other embodiments, a transmembrane domain can include a transmembrane region of an alpha, beta, or zeta chain of a T-cell receptor, such as CD28, CD3, CD45, CD4, CD 8, CD9, CD 16, CD22, CD33, CD37, CD64, CD80, CD86, CD134, CD137 or CD154. In some embodiments, the transmembrane domain can be derived from a CD28 transmembrane domain (CD28tm).

In other embodiments, a polynucleotide herein encoding for a B7H3 CAR can include an intracellular signaling domain linked to a transmembrane domain. In accordance with these embodiments, an intracellular signaling domain can activate a function of a cell when the antigen binding domain binds to a target antigen. In some embodiments, an intracellular signaling domain can activate a function of a cell expressing a CAR, such as a T cell expressing the CAR. In other embodiments, an intracellular signaling domain can contain one or more intracellular signaling domains. In some embodiments, an intracellular signaling domain can include a functional domain of a primary cytoplasmic signaling protein. In some embodiments, an intracellular signaling domain can include a functional domain of a primary cytoplasmic signaling protein, and at least one functional domain of one or more secondary cytoplasmic signaling proteins. In certain embodiments, a primary cytoplasmic signaling protein that acts in a stimulatory manner can contain signaling motifs which are known as intracellular receptor tyrosine-based activation motifs (ITAMs). In accordance with these embodiments, examples of ITAMs containing primary cytoplasmic signaling domains for use herein include, but are not limited to, those derived from CD3 zeta, FcR gamma, CD3 gamma, CD3 delta, CD3 epsilon, CD5, CD22, CD79a, CD79b, or CD66d. In some embodiments, an intracellular signaling domain and/or the co-stimulatory domain herein can include all or a biologically active fragment of CD27, CD28, 41BB, OX40, CD30, CD40, ICOS, lymphocyte function-associated antigen-1 (LFA-1), CD2, CD7, LIGHT, NKG2C, or B7H3, and/or a ligand that specifically binds with CD83. In some embodiments, the intracellular signaling domain herein can include all or a biologically relevant segment of the signaling domain of CD3-zeta or variant thereof and all or a portion of the signaling domain of 4-1BB or variant thereof.

In certain embodiments, provided herein are polynucleotide constructs comprising a first polynucleotide encoding a B7H3 CAR and a second polynucleotide encoding a chemokine receptor. In certain embodiments, the first and second polynucleotides are separate polynucleotides. In certain embodiments, the first and second polynucleotides are the same polynucleotide. In some embodiments, polynucleotide constructs disclosed herein can encode at least one C-X-C chemokine receptor and a B7H3 CAR or a biologically active fragment thereof, wherein the B7H3 CAR can be encoded by a polynucleotide having at least 75%, at least 80%, at least 85% identity (e.g., at least about 85%, at least about 90%, at least about 95%, at least about 99%, up to 100% identity) to a polynucleotide represented by SEQ ID NO: 1, as inserted below. In certain embodiments, the first polynucleotide comprises a sequence at least 75, 80, 85, 90, 95, 96, 97, 98, 99, or 100% identical to SEQ ID NO: 1, preferably 80-100% identical, more preferably 90-100% identical, more preferably 95-100% identical.

(B7H3 CAR)
SEQ ID NO: 1
GCCCGCCACCATGCTGCTGCTGGTGACATCCCTGCTGCTGTGCGA
ACTGCCTCATCCCGCTTTCCTGCTGATTCCTGAAGTCCAGCTGGT
CGAGAGCGGAGGAGGACTGGTGCAGCCTGGAGGATCACTGAGACT
GAGCTGCGCCGCTTCCGGATTCACCTTTAGCTCCTTCGGCATGCA
CTGGGTGAGGCAGGCACCAGGAAAAGGCCTGGAGTGGGTCGCTTA
CATCTCTAGTGACTCAAGCGCCATCTACTATGCAGATACCGTGAA
AGGCAGGTTTACAATCAGTCGCGACAACGCTAAGAATTCCCTGTA
TCTGCAGATGAACTCTCTGCGCGACGAGGATACAGCAGTCTACTA
TTGCGGGGGGGGAAGAGAAAATATCTACTATGGAAGCCGACTGGA
CTACTGGGGACAGGGAACCACAGTGACAGTCTCCTCTGGAGGAGG
AGGAAGCGGAGGAGGAGGATCCGGAGGAGGCGGGTCTGATATCCA
GCTGACTCAGAGCCCCTCCTTCCTGTCTGCCAGTGTGGGCGACAG
GGTCACTATTACCTGTAAGGCATCCCAGAACGTGGATACCAATGT
CGCCTGGTACCAGCAGAAGCCCGGGAAAGCACCTAAGGCCCTGAT
CTATTCAGCCAGCTACCGATATTCTGGCGTGCCAAGTCGGTTCTC
CGGATCTGGCAGTGGGACTGACTTTACACTGACTATTAGTTCACT
GCAGCCCGAAGATTTTGCTACCTACTATTGTCAGCAGTACAATAA
CTACCCATTCACCTTCGGACAGGGGACAAAACTGGAAATCAAAGA
AAGCAAGTACGGACCGCCCTGCCCCCCTTGCCCTGGCCAGCCTAG
AGAACCCCAGGTGTACACCCTGCCTCCCAGCCAGGAAGAGATGAC
CAAGAACCAGGTGTCCCTGACCTGCCTGGTCAAAGGCTTCTACCC
CAGCGATATCGCCGTGGAATGGGAGAGCAACGGCCAGCCCGAGAA
CAACTACAAGACCACCCCCCCTGTGCTGGACAGCGACGGCAGCTT
CTTCCTGTACTCCCGGCTGACCGTGGACAAGAGCCGGTGGCAGGA
AGGCAACGTCTTCAGCTGCAGCGTGATGCACGAGGCCCTGCACAA
CCACTACACCCAGAAGTCCCTGAGCCTGAGCCTGGGCAAGATGTT
CTGGGTGCTGGTGGTGGTCGGAGGCGTGCTGGCCTGCTACAGCCT
GCTGGTCACCGTGGCCTTCATCATCTTTTGGGTGAAACGGGGCAG
AAAGAAACTCCTGTATATATTCAAACAACCATTTATGAGACCAGT
ACAAACTACTCAAGAGGAAGATGGCTGTAGCTGCCGATTTCCAGA
AGAAGAAGAAGGAGGATGTGAACTGCGGGTGAAGTTCAGCAGAAG
CGCCGACGCCCCTGCCTACCAGCAGGGCCAGAATCAGCTGTACAA
CGAGCTGAACCTGGGCAGAAGGGAAGAGTACGACGTCCTGGATAA
GCGGAGAGGCCGGGACCCTGAGATGGGCGGCAAGCCTCGGCGGAA
GAACCCCCAGGAAGGCCTGTATAACGAACTGCAGAAAGACAAGAT
GGCCGAGGCCTACAGCGAGATCGGCATGAAGGGCGAGCGGAGGCG
GGGCAAGGGCCACGACGGCCTGTATCAGGGCCTGTCCACCGCCAC
CAAGGATACCTACGACGCCCTGCACATGCAGGCCCTGCCCCCAAG
G

In some embodiments, polynucleotide constructs disclosed herein can encode at least CXCR1 and a B7H3 CAR where the B7H3 CAR can be encoded by a polynucleotide having at least 75%, at least 80%, at least 85% identity (e.g., at least about 85%, at least about 90%, at least about 95%, at least about 99%, up to 100% identity) to the polynucleotide represented by SEQ ID NO: 1. In some embodiments, polynucleotides disclosed herein can encode CXCR2 and a B7H3 CAR where the B7H3 CAR can be encoded by a polynucleotide having at least 75%, at least 80%, at least 85% identity (e.g., at least about 85%, at least about 90%, at least about 95%, at least about 99%, up to 100% identity) to a polynucleotide represented by SEQ ID NO: 1. In some embodiments, polynucleotides disclosed herein can encode at least CXCR1 and CXCR2 or biologically active fragment thereof, and a B7H3 CAR where the B7H3 CAR can be encoded by a polynucleotide having at least 75%, at least 80%, at least 85% identity (e.g., at least about 85%, at least about 90%, at least about 95%, at least about 99%, up to 100% identity) to the polynucleotide represented by SEQ ID NO: 1. In some embodiments, polynucleotide constructs disclosed herein can encode CXCR2 wherein the CXCR2 can be encoded by a polynucleotide having at least 75%, at least 80%, at least 85% identity (e.g., at least about 85%, at least about 90%, at least about 95%, at least about 99%, up to 100% identity) to a polynucleotide represented by SEQ ID NO: 2 and a B7H3 CAR wherein the B7H3 CAR can be encoded by a polynucleotide having at least 75%, at least 80%, at least 85% identity (e.g., at least about 85%, at least about 90%, at least about 95%, at least about 99%, up to 100% identity) to a polynucleotide represented by SEQ ID NO: 1. In certain embodiments, T cells expressing constructs encoding at least one CXCR and B7H3 CAR having increased honing capabilities to solid tumors than B7H3 CAR T cells not expressing at least one CXCR.

In certain embodiments, polynucleotide constructs contemplated herein or encoded polypeptides contemplated herein (e.g., for transfecting a T cell or NK cell or other lymphocyte cell population) have at least 75%, at least 80%, at least 90%, at least 95% and up to 100% identity to one or more of the following sequences illustrated in Table 1 such as SEQ ID NO. 1, SEQ ID NO. 2, SEQ ID NO. 3, SEQ ID NO. 4, SEQ ID NO. 5, SEQ ID NO. 6. SEQ ID NO. 9, SEQ ID NO. 10, SEQ ID NO. 11. SEQ ID NO. 12, or SEQ ID NO. 13-SEQ ID NO. 16. In certain embodiments, constructs contemplated herein or encoded polypeptides contemplated herein (e.g. for transfecting a T cell or NK cell or other lymphocyte cell population) have at least 75%, at least 80%, at least 90%, at least 95% and up to 100% identity to one or more of SEQ ID NO. 1, SEQ ID NO. 2, SEQ ID NO. 3, SEQ ID NO. 4, SEQ ID NO. 5, SEQ ID NO. 6, SEQ ID NO. 9, SEQ ID NO. 10, SEQ ID NO. 11, and/or SEQ ID NO. 12.

TABLE 1

| | | SEQ |
|---|---|---|
| Name | Sequence | ID NO |
| B7H3 CAR | GCCCGCCACCATGCTGCTGCTGGTGACATCCCTGCTGCTGTGCGA | 1 |
| | ACTGCCTCATCCCGCTTTCCTGCTGATTCCTGAAGTCCAGCTGGT | |
| | CGAGAGCGGAGGAGGACTGGTGCAGCCTGGAGGATCACTGAGACT | |
| | GAGCTGCGCCGCTTCCGGATTCACCTTTAGCTCCTTCGGCATGCA | |
| | CTGGGTGAGGCAGGCACCAGGAAAAGGCCTGGAGTGGGTCGCTTA | |
| | CATCTCTAGTGACTCAAGCGCCATCTACTATGCAGATACCGTGAA | |
| | AGGCAGGTTTACAATCAGTCGCGACAACGCTAAGAATTCCCTGTA | |
| | TCTGCAGATGAACTCTCTGCGCGACGAGGATACAGCAGTCTACTA | |
| | TTGCGGGGGGGGAAGAGAAAATATCTACTATGGAAGCCGACTGGA | |
| | CTACTGGGGACAGGGAACCACAGTGACAGTCTCCTCTGGAGGAGG | |
| | AGGAAGCGGAGGAGGAGGATCCGGAGGAGGCGGGTCTGATATCCA | |
| | GCTGACTCAGAGCCCCTCCTTCCTGTCTGCCAGTGTGGGCGACAG | |
| | GGTCACTATTACCTGTAAGGCATCCCAGAACGTGGATACCAATGT | |
| | CGCCTGGTACCAGCAGAAGCCCGGGAAAGCACCTAAGGCCCTGAT | |
| | CTATTCAGCCAGCTACCGATATTCTGGCGTGCCAAGTCGGTTCTC | |
| | CGGATCTGGCAGTGGGACTGACTTTACACTGACTATTAGTTCACT | |
| | GCAGCCCGAAGATTTTGCTACCTACTATTGTCAGCAGTACAATAA | |
| | CTACCCATTCACCTTCGGACAGGGGACAAAACTGGAAATCAAAGA | |
| | AAGCAAGTACGGACCGCCCTGCCCCCCTTGCCCTGGCCAGCCTAG | |
| | AGAACCCCAGGTGTACACCCTGCCTCCCAGCCAGGAAGAGATGAC | |
| | CAAGAACCAGGTGTCCCTGACCTGCCTGGTCAAAGGCTTCTACCC | |
| | CAGCGATATCGCCGTGGAATGGGAGAGCAACGGCCAGCCCGAGAA | |
| | CAACTACAAGACCACCCCCCCTGTGCTGGACAGCGACGGCAGCTT | |
| | CTTCCTGTACTCCCGGCTGACCGTGGACAAGAGCCGGTGGCAGGA | |
| | AGGCAACGTCTTCAGCTGCAGCGTGATGCACGAGGCCCTGCACAA | |
| | CCACTACACCCAGAAGTCCCTGAGCCTGAGCCTGGGCAAGATGTT | |
| | CTGGGTGCTGGTGGTGGTCGGAGGCGTGCTGGCCTGCTACAGCCT | |
| | GCTGGTCACCGTGGCCTTCATCATCTTTTGGGTGAAACGGGGCAG | |
| | AAAGAAACTCCTGTATATATTCAAACAACCATTTATGAGACCAGT | |
| | ACAAACTACTCAAGAGGAAGATGGCTGTAGCTGCCGATTTCCAGA | |
| | AGAAGAAGAAGGAGGATGTGAACTGCGGGTGAAGTTCAGCAGAAG | |
| | CGCCGACGCCCCTGCCTACCAGCAGGGCCAGAATCAGCTGTACAA | |
| | CGAGCTGAACCTGGGCAGAAGGGAAGAGTACGACGTCCTGGATAA | |
| | GCGGAGAGGCCGGGACCCTGAGATGGGCGGCAAGCCTCGGCGGAA | |
| | GAACCCCCAGGAAGGCCTGTATAACGAACTGCAGAAAGACAAGAT | |
| | GGCCGAGGCCTACAGCGAGATCGGCATGAAGGGCGAGCGGAGGCG | |
| | GGGCAAGGGCCACGACGGCCTGTATCAGGGCCTGTCCACCGCCAC | |
| | CAAGGATACCTACGACGCCCTGCACATGCAGGCCCTGCCCCCAAG | |
| | G | |
| CXCR2 | ATGGAAGATTTTAACATGGAGAGTGACAGCTTTGAAGATTTCTGGA | 2 |
| | AAGGTGAAGATCTTAGTAATTACAGTTACAGCTCTACCCTGCCCCC | |
| | TTTTCTACTAGATGCCGCCCCATGTGAACCAGAATCCCTGGAAATC | |
| | AACAAGTATTTTGTGGTCATTATCTATGCCCTGGTATTCCTGCTGA | |
| | GCCTGCTGGGAAACTCCCTCGTGATGCTGGTCATCTTATACAGCAG | |
| | GGTCGGCCGCTCCGTCACTGATGTCTACCTGCTGAACCTAGCCTTG | |
| | GCCGACCTACTCTTTGCCCTGACCTTGCCCATCTGGGCCGCCTCCA | |
| | AGGTGAATGGCTGGATTTTTGGCACATTCCTGTGCAAGGTGGTCTC | |
| | ACTCCTGAAGGAAGTCAACTTCTATAGTGGCATCCTGCTACTGGCC | |
| | TGCATCAGTGTGGACCGTTACCTGGCCATTGTCCATGCCACACGCA | |
| | CACTGACCCAGAAGCGCTACTTGGTCAAATTCATATGTCTCAGCAT | |
| | CTGGGGTCTGTCCTTGCTCCTGGCCCTGCCTGTCTTACTTTTCCGA | |
| | AGGACCGTCTACTCATCCAATGTTAGCCCAGCCTGCTATGAGGACA | |
| | TGGGCAACAATACAGCAAACTGGCGGATGCTGTTACGGATCCTGCC | |
| | CCAGTCCTTTGGCTTCATCGTGCCACTGCTGATCATGCTGTTCTGC | |
| | TACGGATTCACCCTGCGTACGCTGTTTAAGGCCCACATGGGGCAGA | |
| | AGCACCGGGCCATGCGGGTCATCTTTGCTGTCGTCCTCATCTTCCT | |
| | GCTCTGCTGGCTGCCCTACAACCTGGTCCTGCTGGCAGACACCCTC | |
| | ATGAGGACCCAGGTGATCCAGGAGACCTGTGAGCGCCGCAATCACA | |
| | TCGACCGGGCTCTGGATGCCACCGAGATTCTGGGCATCCTTCACAG | |
| | CTGCCTCAACCCCCTCATCTACGCCTTCATTGGCCAGAAGTTTCGC | |
| | CATGGACTCCTCAAGATTCTAGCTATACATGGCTTGATCAGCAAGG | |
| | ACTCCCTGCCCAAAGACAGCAGGCCTTCCTTTGTTGGCTCTTCTTC | |
| | AGGGCACACTTCCACTACTCTC | |
| T2A peptide | EGRGSLLTCGDVEENPGP | 3 |
| P2A peptide | ATNFSLLKQAGDVEENPGP | 4 |
| E2A peptide | QCTNYALLKLAGDVESNPGP | 5 |
| F2A peptide | VKQTLNFDLLKLAGDVESNPGP | 6 |
| B7H3 R K | CCCTCTCCACTGCCGCTAGCCCTTGGGGGCAGGGCCTG | 7 |
| W (primer) | | |
| RetroF | CTGCCGACCCCGGGGGTGGACCATCCTCTAGACTGCTCGAGGCCCG | 8 |
| (primer) | CCACCATGCTGCTG | |
| CXCR2 | TGCAGGCCCTGCCCCCAAGGGCTAGCGGCAGTGGAGAGGGCAGAGG | 9 |
| gblock | AAGTCTGCTAACATGCGGTGACGTCGAGGAGAATCCTGGCCCAATG | |
| | GAAGATTTTAACATGGAGAGTGACAGCTTTGAAGATTTCTGGAAAG | |
| | GTGAAGATCTTAGTAATTACAGTTACAGCTCTACCCTGCCCCCTTT | |
| | TCTACTAGATGCCGCCCCATGTGAACCAGAATCCCTGGAAATCAAC | |
| | AAGTATTTTGTGGTCATTATCTATGCCCTGGTATTCCTGCTGAGCC | |
| | TGCTGGGAAACTCCCTCGTGATGCTGGTCATCTTATACAGCAGGGT | |
| | CGGCCGCTCCGTCACTGATGTCTACCTGCTGAACCTAGCCTTGGCC | |
| | GACCTACTCTTTGCCCTGACCTTGCCCATCTGGGCCGCCTCCAAGG | |
| | TGAATGGCTGGATTTTTGGCACATTCCTGTGCAAGGTGGTCTCACT | |
| | CCTGAAGGAAGTCAACTTCTATAGTGGCATCCTGCTACTGGCCTGC | |
| | ATCAGTGTGGACCGTTACCTGGCCATTGTCCATGCCACACGCACAC | |
| | TGACCCAGAAGCGCTACTTGGTCAAATTCATATGTCTCAGCATCTG | |
| | GGGTCTGTCCTTGCTCCTGGCCCTGCCTGTCTTACTTTTCCGAAGG | |
| | ACCGTCTACTCATCCAATGTTAGCCCAGCCTGCTATGAGGACATGG | |
| | GCAACAATACAGCAAACTGGCGGATGCTGTTACGGATCCTGCCCCA | |
| | GTCCTTTGGCTTCATCGTGCCACTGCTGATCATGCTGTTCTGCTAC | |
| | GGATTCACCCTGCGTACGCTGTTTAAGGCCCACATGGGGCAGAAGC | |
| | ACCGGGCCATGCGGGTCATCTTTGCTGTCGTCCTCATCTTCCTGCT | |
| | CTGCTGGCTGCCCTACAACCTGGTCCTGCTGGCAGACACCCTCATG | |
| | AGGACCCAGGTGATCCAGGAGACCTGTGAGCGCCGCAATCACATCG | |
| | ACCGGGCTCTGGATGCCACCGAGATTCTGGGCATCCTTCACAGCTG | |
| | CCTCAACCCCCTCATCTACGCCTTCATTGGCCAGAAGTTTCGCCAT | |
| | GGACTCCTCAAGATTCTAGCTATACATGGCTTGATCAGCAAGGACT | |
| | CCCTGCCCAAAGACAGCAGGCCTTCCTTTGTTGGCTCTTCTTCAGG | |
| | GCACACTTCCACTACTCTCTAAGGATCCGATAAAATAAAAGATTTT | |
| | ATTTAGTCTCCAGAAAAAGGG | |
| CXCR1 | MSNITDPQMWDEDDLNFTGMPPADEDYSPCMLETETLNKYVVIIAY | 10 |
| | ALVFLLSLLGNSLVMLVILYSRVGRSVTDVYLLNLALADLLFALTL | |
| | PIWAASKVNGWIFGTFLCKVVSLLKEVNFYSGILLLACISVDRYLA | |
| | IVHATRTLTQKRHLVKFVCLGCWGLSMNLSLPFFLERQAYHPNNSS | |
| | PVCYEVLGNDTAKWRMVLRILPHTFGFIVPLFVMLFCYGFTLRTLF | |
| | KAHMGQKHRAMRVIFAVVLIFLLCWLPYNLVLLADTLMRTQVIQES | |
| | CERRNNIGRALDATEILGFLHSCLNPIIYAFIGQNERHGELKILAM | |
| | HGLVSKEFLARHRVTSYTSSSVNVSSNL | |
| CXCR1 | ATGTCAAATATTACAGATCCACAGATGTGGGATTTTGATGATCTAA | 11 |
| | ATTTCACTGGCATGCCACCTGCAGATGAAGATTACAGCCCCTGTAT | |
| | GCTAGAAACTGAGACACTCAACAAGTATGTTGTGATCATCGCCTAT | |
| | GCCCTAGTGTTCCTGCTGAGCCTGCTGGGAAACTCCCTGGTGATGC | |
| | TGGTCATCTTATACAGCAGGGTCGGCCGCTCCGTCACTGATGTCTA | |
| | CCTGCTGAACCTGGCCTTGGCCGACCTACTCTTTGCCCTGACCTTG | |
| | CCCATCTGGGCCGCCTCCAAGGTGAATGGCTGGATTTTTGGCACAT | |
| | TCCTGTGCAAGGTGGTCTCACTCCTGAAGGAAGTCAACTTCTACAG | |
| | TGGCATCCTGCTGTTGGCCTGCATCAGTGTGGACCGTTACCTGGCC | |
| | ATTGTCCATGCCACACGCACACTGACCCAGAAGCGTCACTTGGTCA | |
| | AGTTTGTTTGTCTTGGCTGCTGGGGACTGTCTATGAATCTGTCCCT | |
| | GCCCTTCTTCCTTTTCCGCCAGGCTTACCATCCAAACAATTCCAGT | |
| | CCAGTTTGCTATGAGGTCCTGGGAAATGACACAGCAAAATGGCGGA | |
| | TGGTGTTGCGGATCCTGCCTCACACCTTTGGCTTCATCGTGCCGCT | |
| | GTTTGTCATGCTGTTCTGCTATGGATTCACCCTGCGTACACTGTTT | |
| | AAGGCCCACATGGGGCAGAAGCACCGAGCCATGAGGGTCATCTTTG | |
| | CTGTCGTCCTCATCTTCCTGCTTTGCTGGCTGCCCTACAACCTGGT | |
| | CCTGCTGGCAGACACCCTCATGAGGACCCAGGTGATCCAGGAGASC | |
| | TGTGAGCGCCGCAACAACATCGGCCGGGCCCTGGATGCCACTGAGA | |
| | TTCTGGGATTTCTCCATAGCTGCCTCAACCCCATCATCTACGCCTT | |
| | CATCGGCCAAAATTTTCGCCATGGATTCCTCAAGATCCTGGCTATG | |
| | CATGGCCTGGTCAGCAAGGAGTTCTTGGCACGTCATCGTGTTACCT | |
| | CCTACACTTCTTCGTCTGTCAATGTCTCTTCCAACCTCTGA | |
| CXCR2 | MEDENMESDSFEDEWKGEDLSNYSYSSTLPPELLDAAPCEPESLEI | 12 |
| | NKYFVVIIYALVFLLSLLGNSLVMLVILYSRVGRSVTDVYLLNLAL | |
| | ADLLFALTLPIWAASKVNGWIFGTFLCKVVSLLKEVNFYSGILLLA | |
| | CISVDRYLAIVHATRTLTQKRYLVKFICLSIWGLSLLLALPVLLER | |
| | RTVYSSNVSPACYEDMGNNTANWRMLLRILPQSFGFIVPLLIMLFC | |
| | YGFTLRTLFKAHMGQKHRAMRVIFAVVLIFLLCWLPYNLVLLADTL | |
| | MRTQVIQETCERRNHIDRALDATEILGILHSCLNPLIYAFIGQKER | |
| | HGLLKILAIHGLISKDSLPKDSRPSFVGSSSGHTSTTL | |
| CXCR1 | ACTCTGATCTCTGACTGCAGCTCCTACTGTTGGACACACCTGGCCG | 13 |
| (nucleotide) | GTGCTTCAGTTAGATCAAACCATTGCTGAAACTGAAGAGGACATGT | |
| | CAAATATTACAGATCCACAGATGTGGGATTTTGATGATCTAAATTT | |
| | CACTGGCATGCCACCTGCAGATGAAGATTACAGCCCCTGTATGCTA | |
| | GAAACTGAGACACTCAACAAGTATGTTGTGATCATCGCCTATGCCC | |
| | TAGTGTTCCTGCTGAGCCTGCTGGGAAACTCCCTGGTGATGCTGGT | |
| | CATCTTATACAGCAGGGTCGGCCGCTCCGTCACTGATGTCTACCTG | |
| | CTGAACCTGGCCTTGGCCGACCTACTCTTTGCCCTGACCTTGCCCA | |
| | TCTGGGCCGCCTCCAAGGTGAATGGCTGGATTTTTGGCACATTCCT | |
| | GTGCAAGGTGGTCTCACTCCTGAAGGAAGTCAACTTCTACAGTGGC | |
| | ATCCTGCTGTTGGCCTGCATCAGTGTGGACCGTTACCTGGCCATTG | |
| | TCCATGCCACACGCACACTGACCCAGAAGCGTCACTTGGTCAAGTT | |
| | TGTTTGTCTTGGCTGCTGGGGACTGTCTATGAATCTGTCCCTGCCC | |
| | TTCTTCCTTTTCCGCCAGGCTTACCATCCAAACAATTCCAGTCCAG | |
| | TTTGCTATGAGGTCCTGGGAAATGACACAGCAAAATGGCGGATGGT | |
| | GTTGCGGATCCTGCCTCACACCTTTGGCTTCATCGTGCCGCTGTTT | |
| | GTCATGCTGTTCTGCTATGGATTCACCCTGCGTACACTGTTTAAGG | |
| | CCCACATGGGGCAGAAGCACCGAGCCATGAGGGTCATCTTTGCTGT | |
| | CGTCCTCATCTTCCTGCTTTGCTGGCTGCCCTACAACCTGGTCCTG | |
| | CTGGCAGACACCCTCATGAGGACCCAGGTGATCCAGGAGAGCTGTG | |
| | AGCGCCGCAACAACATCGGCCGGGCCCTGGATGCCACTGAGATTCT | |
| | GGGATTTCTCCATAGCTGCCTCAACCCCATCATCTACGCCTTCATC | |
| | GGCCAAAATTTTCGCCATGGATTCCTCAAGATCCTGGCTATGCATG | |
| | GCCTGGTCAGCAAGGAGTTCTTGGCACGTCATCGTGTTACCTCCTA | |
| | CACTTCTTCGTCTGTCAATGTCTCTTCCAACCTCTGAAAACCATCG | |
| | ATGAAGGAATATCTCTTCTCAGAAGGAAAGAATAACCAACACCCTG | |
| | AGGTTGTGTGTGGAAGGTGATCTGGCTCTGGACAGGCACTATCTGG | |
| | GTTTTGGGGGGACGCTATAGGATGTGGGGAAGTTAGGAACTGGTGT | |
| | CTTCAGGGGCCACACCAACCTTCTGAGGAGCTGTTGAGGTACCTCC | |
| | AAGGACCGGCCTTTGCACCTCCATGGAAACGAAGCACCATCATTCC | |
| | CGTTGAACGTCACATCTTTAACCCACTAACTGGCTAATTAGCATGG | |
| | CCACATCTGAGCCCCGAATCTGACATTAGATGAGAGAACAGGGCTG | |
| | AAGCTGTGTCCTCATGAGGGCTGGATGCTCTCGTTGACCCTCACAG | |
| | GAGCATCTCCTCAACTCTGAGTGTTAAGCGTTGAGCCACCAAGCTG | |
| | GTGGCTCTGTGTGCTCTGATCCGAGCTCAGGGGGGTGGTTTTCCCA | |
| | TCTCAGGTGTGTTGCAGTGTCTGCTGGAGACATTGAGGCAGGCACT | |
| | GCCAAAACATCAACCTGCCAGCTGGCCTTGTGAGGAGCTGGAAACA | |
| | CATGTTCCCCTTGGGGGTGGTGGATGAACAAAGAGAAAGAGGGTTT | |
| | GGAAGCCAGATCTATGCCACAAGAACCCCCTTTACCCCCATGACCA | |
| | ACATCGCAGACACATGTGCTGGCCACCTGCTGAGCCCCAAGTGGAA | |
| | CGAGACAAGCAGCCCTTAGCCCTTCCCCTCTGCAGCTTCCAGGCTG | |
| | GCGTGCAGCATCAGCATCCCTAGAAAGCCATGTGCAGCCACCAGTC | |
| | CATTGGGCAGGCAGATGTTCCTAATAAAGCTTCTGTTCCGTGCTTG | |
| | TCCCTGTGGAAGTATCTTGGTTGTGACAGAGTCAAGGGTGTGTGCA | |
| | GCATTGTTGGCTGTTCCTGCAGTAGAATGGGGGCAGCACCTCCTAA | |
| | GAAGGCACCTCTCTGGGTTGAAGGGCAGTGTTCCCTGGGGCTTTAA | |
| | CTCCTGCTAGAACAGTCTCTTGAGGCACAGAAACTCCTGTTCATGC | |
| | CCATACCCCTGGCCAAGGAAGATCCCTTTGTCCACAAGTAAAAGGA | |
| | AATGCTCCTCCAGGGAGTCTCAGCTTCACCCTGAGGTGAGCATCAT | |
| | CTTCTGGGTTAGGCCTTGCCTAGGCATAGCCCTGCCTCAAGCTATG | |
| | TGAGCTCACCAGTCCCTCCCCAAATGCTTTCCATGAGTTGCAGTTT | |
| | TTTCCTAGTCTGTTTTCCCTCCTTGGAGACAGGGCCCTGTCGGTTT | |
| | ATTCACTGTATGTCCTTGGTGCCTGGAGCCTACTAAATGCTCAATA | |
| | AATAATGATCACAGGAATGAA | |
| CXCR2 | CAATGTTAGCCCAGCCTGCTATGAGGACATGGGCAACAATACAGCA | 14 |
| (partial | AACTGGCGGATGCTGTTACGGATCCTGCCCCAGTCCTTTGGCTTCA | |
| nucleotide) | TCGTGCCACTGCTGATCATGCTGTTCTGCTACGGATTCACCCTGCG | |
| | TACGCTGTTTAAGGCCCACATGGGGCAGAAGCACCGGGCCATGCGG | |
| | GTCATCTTTGCTGTTGTCCTCATCTTCCTGCTCTGCTGGCTGCCCT | |
| | ACAACCTGGTCCTGCTGGCAGACACCCTCATGAGGACCCAGGTGAT | |
| | CCAGGAGACCTGTGAGCGCCGCAATCACATCGACCGGGCTCTGGAT | |
| | GCCACCGAGATTC | |
| CXCR2 | GGTCATTATCTATGCCCTGGTATTCCTGCTGAGCCTGCTGGGAAAC | 15 |
| (partial | TCCCTCGTGATGCTGGTCATCTTATACAGCAGGGTCGGCTGCTCCG | |
| nucleotide) | TCACTGATGTCTACCTGCTGAACCTAGCCTTGGCCGACCTACTCTT | |
| | TGCCCTGACCTTGCCCATCTGGGCCGCCTCCAAGGTGAATGGCTGG | |
| | ATTTTTGGCACATTCCTGTGCAAGGTGGTCTCACTCCTGAAGGAAG | |
| | TCAACTTCTATAGTGGCATCCTGCTACTGGCCTGCATCAGTGTGGA | |
| | CCGTTACCTGGCCATTGTCCATGCCACACGCACACTGACCCAGAAG | |
| | CGCTACTTGGTCAA | |
| CXCR2 | AGAGACAGAAGGTGGATAGACAAATCTCCACCTTCAGACTGGTAGG | 16 |
| (nucleotide) | CTCCTCCAGAAGCCATCAGACAGGAAGATGTGAAAATCCCCAGCAC | |
| | TCATCCCAGAATCACTAAGTGGCACCTGTCCTGGGCCAAAGTCCCA | |
| | GGACAGACCTCATTGTTCCTCTGTGGGAATACCTCCCCAGGAGGGC | |
| | ATCCTGGATTTCCCCCTTGCAACCCAGGTCAGAAGTTTCATCGTCA | |
| | AGGTTGTTTCATCTTTTTTTTCCTGTCTAACAGCTCTGACTACCAC | |
| | CCAACCTTGAGGCACAGTGAAGACATCGGTGGCCACTCCAATAACA | |
| | GCAGGTCAGGATTTAAGTTTACCTCAAAAATGGAAGATTTTAACAT | |
| | GGAGAGTGACAGCTTTGAAGATTTCTGGAAAGGTGAAGATCTTAGT | |
| | AATTACAGTTACAGCTCTACCCTGCCCCCTTTTCTACTAGATGCCG | |
| | CCCCATGTGAACCAGAATCCCTGGAAATCAACAAGTATTTTGTGGT | |
| | CATTATCTATGCCCTGGTATTCCTGCTGAGCCTGCTGGGAAACTCC | |
| | CTCGTGATGCTGGTCATCTTATACAGCAGGGTCGGCCGCTCCGTCA | |
| | CTGATGTCTACCTGCTGAACCTAGCCTTGGCCGACCTACTCTTTGC | |
| | CCTGACCTTGCCCATCTGGGCCGCCTCCAAGGTGAATGGCTGGATT | |
| | TTTGGCACATTCCTGTGCAAGGTGGTCTCACTCCTGAAGGAAGTCA | |
| | ACTTCTATAGTGGCATCCTGCTACTGGCCTGCATCAGTGTGGACCG | |
| | TTACCTGGCCATTGTCCATGCCACACGCACACTGACCCAGAAGCGC | |
| | TACTTGGTCAAATTCATATGTCTCAGCATCTGGGGTCTGTCCTTGC | |
| | TCCTGGCCCTGCCTGTCTTACTTTTCCGAAGGACCGTCTACTCATC | |
| | CAATGTTAGCCCAGCCTGCTATGAGGACATGGGCAACAATACAGCA | |
| | AACTGGCGGATGCTGTTACGGATCCTGCCCCAGTCCTTTGGCTTCA | |
| | TCGTGCCACTGCTGATCATGCTGTTCTGCTACGGATTCACCCTGCG | |
| | TACGCTGTTTAAGGCCCACATGGGGCAGAAGCACCGGGCCATGCGG | |
| | GTCATCTTTGCTGTCGTCCTCATCTTCCTGCTCTGCTGGCTGCCCT | |
| | ACAACCTGGTCCTGCTGGCAGACACCCTCATGAGGACCCAGGTGAT | |
| | CCAGGAGACCTGTGAGCGCCGCAATCACATCGACCGGGCTCTGGAT | |
| | GCCACCGAGATTCTGGGCATCCTTCACAGCTGCCTCAACCCCCTCA | |
| | TCTACGCCTTCATTGGCCAGAAGTTTCGCCATGGACTCCTCAAGAT | |
| | TCTAGCTATACATGGCTTGATCAGCAAGGACTCCCTGCCCAAAGAC | |
| | AGCAGGCCTTCCTTTGTTGGCTCTTCTTCAGGGCACACTTCCACTA | |
| | CTCTCTAAGACCTCCTGCCTAAGTGCAGCCCCGTGGGGTTCCTCCC | |
| | TTCTCTTCACAGTCACATTCCAAGCCTCATGTCCACTGGTTCTTCT | |
| | TGGTCTCAGTGTCAATGCAGCCCCCATTGTGGTCACAGGAAGTAGA | |
| | GGAGGCCACGTTCTTACTAGTTTCCCTTGCATGGTTTAGAAAGCTT | |
| | GCCCTGGTGCCTCACCCCTTGCCATAATTACTATGTCATTTGCTGG | |
| | AGCTCTGCCCATCCTGCCCCTGAGCCCATGGCACTCTATGTTCTAA | |
| | GAAGTGAAAATCTACACTCCAGTGAGACAGCTCTGCATACTCATTA | |
| | GGATGGCTAGTATCAAAAGAAAGAAAATCAGGCTGGCCAACGGGGT | |
| | GAAACCCTGTCTCTACTAAAAATACAAAAAAAAAAAAAAATTAGCC | |
| | GGGCGTGGTGGTGAGTGCCTGTAATCACAGCTACTTGGGAGGCTGA | |
| | GATGGGAGAATCACTTGAACCCGGGAGGCAGAGGTTGCAGTGAGCC | |
| | GAGATTGTGCCCCTGCACTCCAGCCTGAGCGACAGTGAGACTCTGT | |
| | CTCAGTCCATGAAGATGTAGAGGAGAAACTGGAACTCTCGAGCGTT | |
| | GCTGGGGGGGATTGTAAAATGGTGTGACCACTGCAGAAGACAGTAT | |
| | GGCAGCTTTCCTCAAAACTTCAGACATAGAATTAACACATGATCCT | |
| | GCAATTCCACTTATAGGAATTGACCCACAAGAAATGAAAGCAGGGA | |
| | CTTGAACCCATATTTGTACACCAATATTCATAGCAGCTTATTCACA | |
| | AGACCCAAAAGGCAGAAGCAACCCAAATGTTCATCAATGAATGAAT | |
| | GAATGGCTAAGCAAAATGTGATATGTACCTAACGAAGTATCCTTCA | |
| | GCCTGAAAGAGGAATGAAGTACTCATACATGTTACAACACGGACGA | |
| | ACCTTGAAAACTTTATGCTAAGTGAAATAAGCCAGACATCAACAGA | |
| | TAAATAGTTTATGATTCCACCTACATGAGGTACTGAGAGTGAACAA | |
| | ATTTACAGAGACAGAAAGCAGAACAGTGATTACCAGGGACTGAGGG | |
| | GAGGGGAGCATGGGAAGTGACGGTTTAATGGGCACAGGGTTTATGT | |
| | TTAGGATGTTGAAAAAGTTCTGCAGATAAACAGTAGTGATAGTTGT | |
| | ACCGCAATGTGACTTAATGCCACTAAATTGACACTTAAAAATGGTT | |
| | TAAATGGTCAATTTTGTTATGTATATTTTATATCAATTTAAAAAAA | |
| | AACCTGAGCCCCAAAAGGTATTTTAATCACCAAGGCTGATTAAACC | |
| | AAGGCTAGAACCACCTGCCTATATTTTTTGTTAAATGATTTCATTC | |
| | AATATCTTTTTTTTAATAAACCATTTTTACTTGGGTGTTTATA | |

In certain embodiments, polynucleotide constructs disclosed herein can encode a CAR and CXCR1 wherein the CXCR1 can be encoded by a polynucleotide having at least 75%, at least 80%, at least 90%, at least 95% and up to 100% identity to a polynucleotide represented by SEQ ID NO: 11.

In some embodiments, polynucleotides disclosed herein can encode a CAR that associates with, or binds to, B7H3 and/or CXCR1 or biologically active fragment thereof wherein the CXCR1 can be encoded by a polynucleotide having at least 75%, at least 80%, at least 90%, at least 95% and up to 100% identity to a polynucleotide represented by SEQ ID NO: 11. In certain embodiments, polynucleotide construct for transfection and expression in a cell contemplated herein can encode a CXCR1 having at least 75%, at least 80%, at least 90%, at least 95% and up to 100% identity to a polynucleotide represented by SEQ ID NO: 11 (CXCR1) or biologically active fragment thereof.

In certain embodiments, polynucleotide constructs disclosed herein can encode a CAR and CXCR2 (and CXCR1) where the CXCR2 polypeptide has at least 75%, at least 80%, at least 90%, at least 95% and up to 100% identity to a polypeptide represented by SEQ ID NO: 12. In certain embodiments, polynucleotide constructs disclosed herein can encode a CAR and CXCR1 (and a CXCR2) where the CXCR1 polypeptide has at least 75%, at least 80%, at least 90%, at least 95% and up to 100% identity to a polypeptide represented by SEQ ID NO: 10. It is contemplated that a polynucleotide disclosed herein can encode a polypeptide having at least 75%, at least 80%, at least 90%, at least 95% and up to 100% identity to a polypeptide represented by any CXCR1 and CXCR2 polypeptide alone or in combination with a B7H3 polypeptide. In accordance with these embodiments, a construct can encode a polypeptide having at least 75%, at least 80%, at least 90%, at least 95% and up to 100% identity to a polypeptide SEQ ID NO: 10 and SEQ ID NO: 12.

In some embodiments, polynucleotides can be multicistronic or chimeric polynucleotides and encode more than one polypeptide or peptide fragment thereof (e.g., a B7H3 CAR and a CXCR1 and/or CXCR2). In other embodiments, polynucleotides disclosed herein can include an element to permit translation of multiples genes or gene fragments from a single polynucleotide. In some embodiments, an element can include an internal ribosome entry site (IRES), or a ribosome skip sequence. In some embodiments, an element can include “self-cleaving”2A peptide. In other embodiments, a self-cleaving 2A peptide for use herein can be a T2A peptide (EGRGSLLTCGDVEENPGP; SEQ ID NO: 3), a P2A peptide (ATNFSLLKQAGDVEENPGP; SEQ ID NO: 4), a E2A peptide (QCTNYALLKLAGDVESNPGP; SEQ ID NO: 5), and/or a F2A peptide (VKQTLNFDLLKLAGDVESNPGP; SEQ ID NO: 6) or active fragment thereof. In other embodiments, a self-cleaving 2A peptide represented by at least one of SEQ ID NO: 3-6 can further include GSG residues added to the 5′ end of the peptide to improve cleavage efficiency.

Certain embodiments of the present disclosure relate to polypeptides comprising chimeric antigen receptors (CAR) or biologically active fragments thereof and chemokine receptors, such as a C-C motif and/or C-X-C motif chemokine receptor. In certain embodiments, a polypeptide comprises a CAR and a chemokine receptor linked via a polypeptide linker. The polypeptide can comprise any suitable CAR. In certain embodiments, the CAR is a B7H3 CAR. In certain embodiments, the CAR polypeptide comprises a sequence at least 70, 75, 80, 85, 90, 95, 96, 97, 98, 99, 99.5, or 100% identical to a polypeptide encoded by SEQ ID NO: 1. The polypeptide can comprise any suitable chemokine receptor. In certain embodiments, the chemokine receptor is a CCR or a CCR receptor, for example CXCR1 or CXC2. In certain embodiments, the CXCR1 polypeptide comprises a sequence at least 70, 75, 80, 85, 90, 95, 96, 97, 98, 99, 99.5, or 100% identical to a polypeptide encoded by SEQ ID NO: 10. In certain embodiments, the CXCR2 polypeptide comprises a sequence at least 70, 75, 80, 85, 90, 95, 96, 97, 98, 99, 99.5, or 100% identical to a polypeptide encoded by SEQ ID NO: 12. In certain embodiments, the polypeptide peptide comprises a B7H3 CAR and CXCR1 linked via a polypeptide linker. In certain embodiments, the polypeptide comprises a B7H3 CAR and a CXCR2 linked via a polypeptide linker. In certain embodiments, the polypeptide linker comprises a self-cleaving peptide. Any suitable self-cleaving peptide can be used, such as a 2A peptide, for example a T2A peptide (EGRGSLLTCGDVEENPGP; SEQ ID NO: 3), a P2A peptide (ATNFSLLKQAGDVEENPGP; SEQ ID NO: 4), a E2A peptide (QCTNYALLKLAGDVESNPGP; SEQ ID NO: 5), and/or a F2A peptide (VKQTLNFDLLKLAGDVESNPGP; SEQ ID NO: 6) or active fragment thereof. In other embodiments, a self-cleaving 2A peptide represented by at least one of SEQ ID NO: 3-6 can further include GSG residues added to the 5′ end of the peptide to improve cleavage efficiency.

In certain embodiments, compositions and methods provided herein can include a vector containing one or more polynucleotides disclosed herein. In some embodiments, a vector for use herein can be a viral vector or a baboon pseudotyped vector or other vector.

As used herein, the term “viral vector” can refer to a nucleic acid vector construct that includes at least one element of viral origin and has the capacity to be packaged into a viral vector particle and encodes at least an exogenous polynucleotide. In certain embodiments, the vector and/or particle can be utilized for the purpose of transferring any nucleic acids into cells either in vitro or in vivo. Numerous viral vectors are known in the art. The term virion can refer to a single infective viral particle. “Viral vector”, “viral vector particle” and “viral particle” also refer to a complete virus particle with its DNA or RNA core and protein coat as it exists outside the cell. Non-limiting examples of viral vectors for use herein can include adenoviruses, adeno-associated viruses (AAV), herpesviruses, retroviruses, lentiviruses, integrase defective lentiviruses (IDLV), and the like. In some embodiments, a viral vector disclosed herein can be a lentiviral vector. Examples of lentiviruses include, but are not limited to, human lentiviruses such as HIV (in particular HIV-1 or HIV-2), simian immunodeficiency virus (SIV), equine infectious anemia virus (EIAV), feline immunodeficiency virus (FIV), Caprine Arthritis Encephalitis Virus (CAEV), visna and progressive pneumonia viruses of sheep, baboon pseudotyped viruses, bovine immunodeficiency virus (BIV), and the like.

In some embodiments, polynucleotides and/or vectors described herein can be prepared by conventional recombinant technology known to one of skill in the art. In other embodiments, polynucleotides and/or vectors described herein can be prepared by a gene editing methods known in the art (e.g., by CRISPR).

In certain embodiments, methods provided herein can include generating a cell to express any of the polynucleotides and/or vectors described herein. In some embodiments, cells for use herein can be one or more immune cells. As used herein an “immune cell” can refer to a cell of the immune system. Immune cells can be categorized as lymphocytes, neutrophils, granulocytes, mast cells, monocytes/macrophages, and dendritic cells. In some embodiments, cells for use herein can be one or more lymphocytes. In some embodiments, lymphocytes can include T-cells (CD4 T cells and/or CD8 T cells), B-cells, and/or natural killer (NK) cells and transfection thereof. In some embodiments, cells for use herein can be one or more cytotoxic lymphocytes. As used herein, a “cytotoxic lymphocyte” refers to a lymphocyte capable cytolysis. For example, but not limited to, a cytotoxic lymphocyte can be capable of killing cancer cells, cells that are infected (particularly with viruses), and cells that are damaged in one or more other ways.

In certain embodiments, the cell comprises a eukaryotic cell. In certain embodiments, the cell comprises a human cell. In certain embodiments, the human cell comprises a human immune cell or a human stem cell. The human immune cell can be any suitable human immune cell such as a neutrophil, eosinophil, basophil, mast cell, monocyte, macrophage, dendritic cell, natural killer cell, or a lymphocyte, preferably a natural killer (NK) cell, more preferably a T cell, even more preferably a CD8+ T cell, a CD4+ T cell, a CD8+/CD4+ double positive T cell, or a combination thereof. In certain embodiments, the CD8+ T cell comprises a naive CD8+ T cell, a central memory CD8+ T cell, an effector memory CD8+ T cell, a bulk CD8+ T cell, or a combination thereof. In certain embodiments, the CD4+ T cell comprises a naive CD4+ T cell, a central memory CD4+ T cell, an effector memory CD4+ T cell, a bulk CD4+ T cell, or a combination thereof. In certain embodiments, the T cell comprises a precursor T cell. The human stem cell can be any suitable human stem cell such as a human pluripotent, multipotent stem cell, embryonic stem cell, induced pluripotent stem cell, hematopoietic stem cell, CD34+ cell preferably a hematopoietic stem cell, more preferably an induced pluripotent stem cell.

In certain embodiments, a polynucleotide or a polynucleotide construct as described herein can be introduced into the genome of a cell. Any suitable genome editing technology can be used, such as a CRISPR-cas system, for example a Class 1 or a Class 2 system, e.g., Cas9 or Cas12a. In certain embodiments, the CRISPR-cas system comprises a cas nuclease and/or a guide nucleic acid, wherein the cas nuclease and the guide nucleic acid for a nucleic acid-guided nuclease complex, i.e., a ribonucleoprotein (RNP), wherein the nucleic acid-guided nuclease system is capable at binding to a site at least partially complementary to the spacer sequence of the guide nucleic acid and generate one or more strand breaks in a target polynucleotide. The nucleic acid-guided nuclease complex can be delivered to the cell as a fully formed complex, as individual components, and/or as one or more polynucleotides encoding for one or more components of the nucleic acid-guided nuclease complex. Upon delivery to the cell with a suitable polynucleotide or polynucleotide construct as described above, the nucleic acid-guided nuclease complex can generate one or more strand breaks at or near a target site in the genome of a cell, wherein at least a portion of the polynucleotide or polynucleotide construct can be introduced at or near the strand break by an innate repair mechanism, such as homology directed repair (HDR).

In some embodiments, cells for use herein can be isolated from a subject. In some embodiments, cells for use herein can be isolated from peripheral blood, umbilical cord blood, and/or bone marrow. In some embodiments, cells for use herein can be isolated from peripheral blood mononuclear cells (PBMCs). In some embodiments, cells for use herein can be isolated from a leukapheresis sample. In some embodiments, cells for use herein can be isolated from tumor-infiltrated lymphocytes, tissue-infiltrated lymphocytes, lymph nodes, thymus, and/or secondary lymphoid organs. In some embodiments, cells for use herein can be isolated from autologous peripheral blood, umbilical cord blood, bone marrow, PBMCs, leukapheresis sample, tumor-infiltrated lymphocytes, tissue-infiltrated lymphocytes, lymph nodes, thymus, and/or secondary lymphoid organs. As used herein, the term “autologous” refers to peripheral blood, umbilical cord blood, bone marrow, PBMCs, leukapheresis sample, tumor-infiltrated lymphocytes, tissue-infiltrated lymphocytes, lymph nodes, thymus, and/or secondary lymphoid organs obtained from the same subject to be treated with the compositions disclosed herein. In some embodiments, cells for use herein can be isolated from allogeneic peripheral blood, umbilical cord blood, bone marrow, PBMCs, leukapheresis sample, tumor-infiltrated lymphocytes, tissue-infiltrated lymphocytes, lymph nodes, thymus, and/or secondary lymphoid organs. As used herein, the term “allogeneic” refers to peripheral blood, umbilical cord blood, bone marrow, PBMCs, leukapheresis sample, tumor-infiltrated lymphocytes, tissue-infiltrated lymphocytes, lymph nodes, thymus, and/or secondary lymphoid organs obtained from a different subject of the same species as the subject to be treated with the compositions disclosed herein. In some embodiments, cells for use herein can be isolated from haploidentical allogeneic peripheral blood, umbilical cord blood, bone marrow, PBMCs, leukapheresis sample, tumor-infiltrated lymphocytes, tissue-infiltrated lymphocytes, lymph nodes, thymus, and/or secondary lymphoid organs.

In other embodiments, methods and compositions provided herein can include a cell having a vector and/or a polynucleotide encoding a B7H3 CAR and at least one CXCR (e.g., CXCR1 and/or CXCR2). In some embodiments, methods herein can include introducing any vector and/or a polynucleotide disclosed herein into a lymphocyte, culturing the lymphocyte encompassing the vector and/or a polynucleotide disclosed herein in the presence of an agent in the culture medium that promotes expansion of a cell population expressing a vector and/or a polynucleotide disclosed herein, and selectively enriching for the lymphocyte expressing vector and/or a polynucleotide disclosed herein. Non-limiting examples of such agents for use in a culture medium as disclosed herein can include an anti-CD3 antibody, an anti-CD28 antibody, a cytokine, and the like. In some embodiments, cytokines for use herein can be IL-15, IL-7, IL-2, IL-12, IL-18, IL-21, or a combination thereof. In some embodiments, selectively enriching can be performed by contacting a lymphocyte expressing vector and/or a polynucleotide disclosed herein with one or more a selection reagent. In some embodiments, a selection reagent can be methotrexate. In some embodiments, lymphocytes for use herein can have a CD45RA-, CD45RO+, and CD62L+ phenotype. In some embodiments, lymphocytes for use herein can be CD8+ lymphocytes or CD4+ lymphocytes.

In certain embodiments, cells expressing any of the polynucleotides and/or vectors described herein can include one or more T lymphocyte populations. In some embodiments, cells herein can be selected for and/or sorted into one or more T lymphocyte populations of use in methods for transforming the population of cells for therapeutic use. In certain embodiments selection and sorting of T lymphocyte populations, T lymphocytes can be collected in accordance with known techniques in the art. In certain embodiments, T cells can be selected that display one or more markers such as CD4 or CD8 or a combination thereof.

In some embodiments, a T cell population or subpopulation of cells disclosed herein can be expanded for example, alone or by introducing an initial T lymphocyte population to a culture medium in vitro, and providing culture medium feeder cells, such as non-dividing peripheral blood mononuclear cells (PBMC). In some embodiments, PBMC feeder cells can be irradiated with gamma rays. In other embodiments, an expansion method, for example for NK cells or transfected NK cells, can include adding non-dividing EBV-transformed lymphoblastoid cells (LCL) as feeder cells. In certain embodiments, LCL feeder cells can be irradiated with gamma rays. In other embodiments, an expansion method can include adding anti-CD3 and/or anti CD28 antibody to a culture medium. In some embodiments, an expansion method can include adding one or more of IL-15, IL-7, IL-2, IL-12, IL-18, IL-21, or a combination thereof to a culture medium (e.g., wherein the concentration is at least about 1 to about 30 units/ml or about 10 units/ml). In some embodiments, the expanded T lymphocytes can include CD8+ cytotoxic T lymphocytes (CTL) and CD4+ helper T lymphocytes that can be specific for an antigen present on a human tumor or a pathogen. After initial isolation of T lymphocytes both cytotoxic and helper T lymphocytes can be sorted into naive, memory, and effector T cell subpopulations either before or after expansion. In some embodiments, a T cell population or subpopulation of cells herein can be CD8+ T cells. In accordance with certain embodiments disclosed herein, CD8+ T cells can be naive CD8+ T cells, central memory CD8+ T cells, effector memory CD8+ T cells, bulk CD8+ T cells, or a combination thereof. In some embodiments, a T cell population or subpopulation of T cells disclosed herein can be CD4+ T cells. In other embodiments, CD4+ T cells can be naive CD4+ T cells, central memory CD4+ T cells, effector memory CD4+ T cells, bulk CD4+ T cells, or a combination thereof. In some embodiments, cells expressing any of the polynucleotides and/or vectors disclosed herein can be precursor T cells. As used herein, T cell precursors can be used for transforming, expanding, or developing into a population of T cells for use for in treating solid tumors, other tumors, or other malignancies. In some embodiments, lymphoid precursor cells that can migrate to the thymus and become T cell precursors, which do not express a T cell receptor can be used. T cells originate from hematopoietic stem cells in the bone marrow. Hematopoietic progenitors (lymphoid progenitor cells) from hematopoietic stem cells populate the thymus and expand by cell division to generate a population of immature thymocytes. The earliest thymocytes express neither CD4 nor CD8 and are therefore classed as double-negative (CD4⁻CD8⁻) cells. As they progress through their development, they become double-positive thymocytes (CD4⁺CD8⁺), and finally mature to single-positive (CD4⁺CD8⁻ or CD4⁻CD8⁺) thymocytes that are then released from the thymus to peripheral tissues. In some embodiments, cells expressing any of the polynucleotides and/or vectors described herein can be hematopoietic stem cells able to mature into T cells of use herein. The precursor can be any suitable stem cell, for example an induced pluripotent stem cell or a hematopoietic stem cell.

In certain embodiments, pharmaceutical compositions are contemplated of use herein. In accordance with these embodiments, pharmaceutical compositions can include one or more of the polynucleotides, polypeptides, vectors-containing constructs disclosed herein, and/or T cells (e.g., CAR T cells) expressing constructs disclosed herein. In some embodiments, pharmaceutical compositions can include one or more of the polynucleotides, polypeptides, vectors-containing constructs disclosed herein, and/or T cells (e.g., CAR T cells) expressing constructs disclosed herein and at least one pharmaceutically acceptable excipient or carrier. As used herein, the term “pharmaceutically acceptable” refers to those compounds, materials, agents, compositions, and/or dosage forms which are, within the scope of sound medical judgment, suitable for use in contact with the tissues, organs, and/or bodily fluids of a subject without excessive toxicity, irritation, allergic response, or other problems or complications commensurate with a reasonable benefit/risk ratio. As used herein, the term “pharmaceutically acceptable carrier” can refer to solvents, dispersion media, coatings, antibacterial agents, antifungal agents, isotonic and absorption delaying agents, or the like that are physiologically compatible. Pharmaceutically acceptable carriers suitable for use herein, can include, but are not limited to, buffers that are well known in the art, and can be phosphate, citrate, tris, histidine or other amino acid buffer or other organic acids. In other embodiments, buffers can include, but are not limited to, antioxidants including ascorbic acid and methionine; preservatives; low molecular weight polypeptides; proteins, such as serum albumin, gelatin, or immunoglobulins; amino acids; hydrophobic polymers; monosaccharides; disaccharides; and other carbohydrates; metal complexes; and/or non-ionic surfactants.

In some embodiments, pharmaceutical compositions of use disclosed herein can be formulated for parenteral administration, such as intravenous or intravascular, bolus infusion, intrarenal introduction, intracerebroventricular injection, intra-cisterna magna injection, intra-parenchymal injection, direct introduction to a tumor or tumors or a combination thereof. In some embodiments, pharmaceutical compositions for use herein can be formulated for local delivery to one or more tumors. In some embodiments, pharmaceutical compositions for use herein be formulated for parenteral administration can include pharmaceutically acceptable carriers including sterile liquids, such as water and oil, including those of petroleum, animal, vegetable, or synthetic origin, such as peanut oil, soybean oil, mineral oil, and the like. Saline solutions and aqueous dextrose, polyethylene glycol (PEG) and glycerol solutions can also be employed as liquid carriers, particularly for injectable solutions. In some embodiments, pharmaceutical compositions for use herein can further include additional ingredients, for example preservatives, buffers, tonicity agents, antioxidants and stabilizers, nonionic wetting or clarifying agents, viscosity-increasing agents, and the like. In some embodiments, pharmaceutical compositions described herein can be packaged in single unit dosages or in multi-dosage forms.

In some embodiments, formulations suitable for parenteral administration include aqueous and non-aqueous sterile injection solutions which can contain antioxidants, buffers, bacteriostats and solutes which render the formulation isotonic with the blood of the intended recipient; and aqueous and non-aqueous sterile suspensions which can include suspending agents and thickening agents. In accordance with some embodiments herein, aqueous solutions can be suitably buffered (e.g., a pH of about 3.0 to 9.0 or above). In some embodiments, pharmaceutical compositions disclosed herein containing CAR T cells contemplated herein include conditions suitable for cell preservation to reduce apoptosis, degradation, or other negative affect on the CAR T cell populations. Preparation of suitable parenteral formulations for use herein under sterile conditions can be readily accomplished by standard pharmaceutical techniques well known to those skilled in the art.

In some embodiments, pharmaceutical compositions herein can further include one or more pharmaceutically acceptable salts. Non-limiting examples of pharmaceutically acceptable salts include acid addition salts (formed from a free amino group of a polypeptide with an inorganic acid, or an organic acid. In some embodiments, the salt formed with the free carboxyl groups is derived from an inorganic base, or an organic base. In some embodiments, pharmaceutical compositions disclosed herein can include a population of the genetically engineered or transfected CAR-T cells disclosed herein (e.g., CAR-T cells expressing B7H3 and CXCR1/CXCR2, CXCR1 or CXCR2 alone, or other CXCR or other construct) suspended in a cryopreservation solution (e.g., CryoStor C55), frozen in a cryopreservation solution or thawed in a cryopreservation solution for later therapeutic use. In some embodiments, any of the pharmaceutical compositions herein can be used in therapeutic applications, for example, to treat one or more of, a solid tumor, other tumor, or malignancies in a subject, which are disclosed herein.

In certain embodiments, methods of treating or ameliorating cancer, a tumor, other malignancy, or a combination thereof in a subject are disclosed. In some embodiments, methods of treating or ameliorating a solid tumor, other tumor or malignancy in a subject include, but are not limited to, administration of an effective amount of any the polynucleotides, polypeptides, vectors-containing constructs disclosed herein, and/or cells containing constructs or expressing constructs and/or pharmaceutical compositions (e.g., CAR T cell therapy) containing these agents thereof described herein. “An effective amount” as used herein refers to a dose of CAR T cell therapy or number of CAR T cells needed that is sufficient to confer a therapeutic effect on a subject having or suspected of having cancer, a tumor, or any combination thereof and further treating the cancer. In certain embodiments, a therapeutic effect for a subject having or suspected of having a tumor can include reducing the symptoms or consequences of the cancer, such as reducing expansion of, shrinking of a tumor, killing tumor cells, preventing the occurrence of metastases from a primary tumor, reducing the number of tumor cells of a tumor, primary tumor and/or a metastatic tumor, inhibiting the growth of tumor cells of a primary tumor and/or a metastatic tumor, eliminating tumor cells in a subject by killing the cells or preventing propagation or expansion of the solid tumor cells and the like.

In some embodiments, methods of administering a CAR T cell therapy as disclosed herein can include placement (e.g., transplantation or implantation) of any the polynucleotides, polypeptides, vectors-containing constructs disclosed herein, and/or T cells (e.g. CAR T cells) expressing constructs disclosed herein or pharmaceutical compositions containing these agents into a subject, by a method or route that results in at least partial localization of the introduced cells at a desired site, such as tumor, such that a desired effect(s) is produced. In certain embodiments, CAR T cell therapy disclosed herein can be administered by any appropriate route that results in delivery to a desired location in the subject where at least a population of the implanted cells or components of the cells remain viable and directed to a targeted solid tumor, other tumor, or other malignancy. In certain embodiments, a bolus of CAR T cells or NK cells administered to a subject can be from about 1-100×10⁶/kg; about 1-50×10⁶/kg or about 1-10×10⁶/kg or other suitable number considering the condition of the subject and the subject to be treated, for example.

In some embodiments, a subject can be transfused with therapeutic T cells disclosed herein over the course of a day, for a few hours, daily, every other day, 2 times per week, weekly, every other week, monthly, or other appropriate treatment regimen. In accordance with the embodiments herein, the period of viability of the cells herein after administration to a subject can be a few hours (e.g., about 2 hours, about 6 hours, about 12 hours, about 24 hours), a few days (e.g., about 1 day, about 2 days, about 3 days, about 4 days about 5 days, about 6 days, about 7 days), weeks (e.g., about 2 weeks, about 4 weeks, about 6 weeks, about 12 weeks, about 40 weeks, about 52 weeks), to as long as several years (e.g., about 2 years, about 5 years), or even the life time of the subject, i.e., long-term engraftment. In some embodiments, an effective amount of the therapeutic T cells herein can be administered via a systemic route of administration, such as an intraperitoneal or intravenous route. In other embodiments, these regimens can be combined with standard cancer therapies to reduce or eliminate solid tumors in a subject (e.g., radiation, chemotherapy, or surgery).

In some embodiments, a subject of any of the methods herein can be any subject for whom treatment or therapy is desired or projected to be effective. In some embodiments, a subject can have or can be suspected of developing cancer or have a solid tumor or have another tumor or have another malignancy or other health condition in need of treatment. In some embodiments, a subject can have or can be suspected of having one or more primary tumors, one or more metastatic tumors such as solid tumors, other tumors, other malignancy, or a combination thereof. In some embodiments, a subject can be a mammal. In other embodiments, a subject can be a pet, livestock, a horse, or a human patient. In some embodiments, a human patient such as an adult, child, adolescent, toddler, young adult or infant or fetus who is in need of the methods disclosed herein can be identified by routine medical examination, e.g., laboratory tests, biopsy, magnetic resonance imaging (MRI) scans, ultrasound exams, and the like.

In other embodiments, a subject to be treated by the methods described herein can be a human patient having, suspected of having, or a risk for developing a solid tumor, other tumor, or other malignancy. In some embodiments, a subject to be treated by the methods described herein can have a tumor or other malignancy where the tumor has at least one B7H3⁺ expressing cell. In other embodiments, a subject to be treated by the methods described herein can have a tumor or other malignancy where the tumor or other malignancy has at least one IL-8⁺ expressing cell. In other embodiments, a subject to be treated by the methods described herein can have a tumor or other malignancy where the tumor has at least one IL-8⁺ cell and at least one B7H3⁺ cell. In some embodiments, a subject to be treated by compositions and methods described herein can have a tumor or malignancy where the tumor has at least one IL-8⁺ cell+/B7H3⁺ cell.

In some embodiments, a subject to be treated by compositions and methods disclosed herein can have or be suspected of developing a sarcoma. In accordance with these embodiments, a sarcoma to be treated by compositions and methods disclosed herein (e.g., transfected CAR T cells) can be a bone sarcoma, a soft-tissue sarcoma, or other sarcoma, or a combination thereof. In some embodiments, bone sarcoma suitable for treatments disclosed herein include, but are not limited to, osteosarcoma, chondrosarcoma, poorly differentiated round/spindle cell tumors, Ewing sarcoma, hemangioendothelioma, angiosarcoma, fibrosarcoma/myofibrosarcoma, chordoma, adamantinoma, liposarcoma, leiomyosarcoma, malignant peripheral nerve sheath tumor, rhabdomyosarcoma, synovial sarcoma, malignant solitary fibrous tumor, or any combination thereof. In some embodiments, a subject to be treated by the therapeutic compositions and methods disclosed herein can have or be suspected of having osteosarcoma. Non-limiting examples of soft-tissue sarcoma suitable for methods herein include fibrous tumor, inflammatory myofibroblastic tumor, low-grade myofibroblastic sarcoma, fibrosarcoma, myxofibrosarcoma, low-grade fibromyxoid sarcoma, giant cell tumor of soft tissues, leiomyosarcoma, malignant glomus tumor, rhabdomyosarcoma, hemangioendothelioma, angiosarcoma of soft tissue, extraskeletal osteosarcoma, gastrointestinal stromal tumor, malignant, malignant peripheral nerve sheath tumor, malignant Triton tumor, malignant granular cell tumor, malignant ossifying fibromyxoid tumor, stromal sarcoma not otherwise specified, myoepithelial carcinoma, malignant phosphaturic mesenchymal tumor, synovial sarcoma, epithelioid sarcoma, alveolar soft part sarcoma, clear cell sarcoma of soft tissue, extraskeletal myxoid chondrosarcoma, extraskeletal Ewing sarcoma, desmoplastic small round cell tumor, extrarenal rhabdoid tumor, perivascular epithelioid cell tumor, intimal sarcoma, undifferentiated spindle cell sarcoma, undifferentiated pleomorphic sarcoma, undifferentiated round cell sarcoma, undifferentiated epithelioid sarcoma, undifferentiated sarcoma, not otherwise specified, or any combination thereof. In some embodiments, a subject to be treated by the compositions and methods disclosed herein can have or be suspected of having rhabdomyosarcoma.

In some embodiments, combination therapies are contemplated and can include administering any polynucleotide construct, vector containing one or more of the polynucleotide constructs, T cells transfected with one or more polynucleotide construct and/or pharmaceutical compositions (e.g., CAR T cell therapy) described herein in combination with one or more of radiation therapy, before, at the same time or after compositions disclosed herein are delivered to a subject. In some embodiments, a subject can be treated by radiation therapy to condition the subject for a CAR T cell therapy disclosed herein where the subject can be irradiated and then treated with CAR T therapy disclosed herein. In other embodiments, a subject can be treated by radiation therapy to increase IL-8 expression on the tumor or malignancy before administration of a CAR T cell therapy disclosed herein. In some embodiments, a subject can be treated by radiation therapy to increase IL-8 expression by at least about 2-fold, about 5-fold, or about 10-fold before administration of the CAR T cell therapy disclosed herein. In some embodiments, a subject can be treated by radiation therapy at least 12 hours, about 24 hours, about 2 days, about 3 days, about 4 days, about 5 days, about 6 days, about 1 week, or about 2 weeks before administration of a CAR T cell therapy disclosed herein. In other embodiments, a subject can be treated by radiation therapy using ionizing radiation. In some embodiments, a subject can be treated by radiation therapy delivered by a linear accelerator. In certain embodiments, a subject can be treated by radiation therapy delivered directly to a tumor. In some embodiments, radiation therapy can be delivered directly to a tumor (e.g., a solid tumor) at a dose of radiation ranging from about 2 Gy to about 50 Gy (e.g., about 2, about 5, about 10, about 20, about 30, about 40, about 50 Gy or other suitable radiation dose).

In other embodiments, combination treatments with transfected CAR Ts and/or NK cells disclosed herein can include radiation therapy, chemotherapy and/or surgery. In certain embodiments, a subject can undergo surgery to remove a portion of the tumor and then be treated with CAR T and/or NK cells expressing one or more construct contemplated herein. In certain embodiments, treatments of a tumor or other malignancy can include combinations of transfected CAR T and NK cells in a single administration, altering or simultaneous administration for example.

In certain embodiments, a subject treated with any of the methods herein can have completed an additional therapeutic regimen, be receiving an additional therapeutic regimen, or can receive an additional therapeutic regimen following treatment disclosed herein. In some embodiments, an additional therapeutic regimen for use herein can include administering a chemotherapeutic agent. In some embodiments, a chemotherapeutic agent can be a cell cycle inhibitor. As used herein “cell cycle inhibitor” can include a chemotherapeutic agent that inhibits or prevents the division and/or replication of cells. In some embodiments, a cell cycle inhibitor can include a chemotherapeutic agent including, but not limited to, Doxorubicin, Melphlan, Roscovitine, Mitomycin C, Hydroxyurea, 5-Fluorouracil, Cisplatin, Ara-C, Etoposide, Gemcitabine, Bortezomib, Sunitinib, Sorafenib, Sodium Valproate, a HDAC Inhibitor, or Dacarbazine. More examples of additional chemotherapeutic agents include, but are not limited to, HDAC inhibitors such as FR01228, Trichostatin A, SAHA and/or PDX101. In some embodiments, the cell cycle inhibitor is a DNA synthesis inhibitor. As used herein, a “DNA synthesis inhibitor” can include a chemotherapeutic agent that inhibits or prevents the synthesis of DNA by a cancer cell. Examples of DNA synthesis inhibitors include, but are not limited to, AraC (cytarabine), 6-mercaptopurine, 6-thioguanine, 5-fluorouracil, capecitabine, floxuridine, gemcitabine, decitabine, vidaza (aza), fludarabine, nelarabine, cladribine, clofarabine, pentostatin, thiarabine, troxacitabine, sapacitabine or forodesine. More examples of additional chemotherapeutic agents include, but are not limited to, FLT3 inhibitors such as Semexanib (SCT5416), Sunitinib (SU 11248), Midostaurin (PKC412), Lestautinib (CEP-701), Tandutinib (MLN518), CHIR-258, Sorafenib (BAY-43-9006) and/or KW-2449. More non-limiting examples of additional chemotherapeutic agents include farnesyltransferase inhibitors such as tipifarnib (RI 15777, Zarnestra), lonafarnib (SCH66336, Sarasar™) and/or BMS-214662. More examples of additional chemotherapeutic agents include, but are not limited to, topoisomerase II inhibitors such as the epipodophyllotoxins etoposide, teniposide, anthracyclines doxorubicin and/or 4-epi-doxorubicin. More non-limiting examples of additional chemotherapeutic agents include P-glycoprotein modulators such as zosuquidar trihydrochloride (Z.3HCL), vanadate, or verapamil. More non-limiting examples of additional chemotherapeutic agents include, but are not limited to, hypomethylating agents such as 5-aza-cytidine or 2′ deoxyazacitidine.

In certain embodiments, polynucleotides, polypeptides, vectors-containing constructs disclosed herein, and/or cells expressing constructs and/or pharmaceutical compositions (e.g., CAR T cell therapy) described herein can be administered in dosages and by techniques well known to those skilled in the medical and veterinary arts. In accordance with these embodiments, healthcare professions will take into consideration such factors as the age, sex, weight, and condition of the specific patient to be treated, and the composition form used for administration (e.g., solid vs. liquid). Dosages for humans or other mammals can be determined without undue experimentation by the skilled artisan, from this disclosure, and the knowledge in the art.

In certain embodiments, provided herein are methods for killing a target cell comprising contacting the target cell with a cell of any one of the embodiments described above. In certain embodiments, the target cell comprises surface expressed B7H3. In certain embodiments, the target cell further secretes a cytokine, such as a chemokine, for example an interleukin, e.g., IL-1, 2, 3, 5, 6, 7, 8, or a combination thereof. An illustrative example includes providing a composition comprising an engineered T cell comprising a polynucleotide construct encoding a B7H3 CAR and CXCR2, wherein the B7H3 CAR and the CXCR2 are expressed on the surface of the cell, to a sample comprising a target cell comprising surface-expressed B7H3 and secreted IL-8. The engineered T cell, upon binding to IL-8 homes towards the target cell in response to the IL-8 gradient, the B7H3 CAR on the engineered T cell binds to the B7H3 antigen on the target cell, whereby the engineered T cell stimulates cell death of the target cell through one or more mechanisms.

In some embodiments, kits are contemplated of use to generate transfected cells disclosed herein. In other embodiments, kits can include therapeutic cell populations where the therapeutic cell populations can be used immediately or frozen and stored for transport and later use. In other embodiments, a kit can include any of the polynucleotides. polypeptides, vectors-containing constructs disclosed herein, and/or cells containing constructs or expression constructs, or pharmaceutically acceptable formulation disclosed herein. In some embodiments, a kit can further include T cells disclosed herein and/or buffers/reagents needed to collect and culture any of the immune cells disclosed herein. In certain embodiments, T cells can be generated from a patient and transfected for re-introduction to that patient, personalized.

In some embodiments, a kit can further include any lymphocytes contemplated herein and/or buffers/reagents needed to collect and/or culture any of the lymphocytes of use herein for expansion and later use. In some embodiments, a kit can further include a culture medium including, but not limited to, an anti-CD3 antibody, an anti-CD28 antibody, a cytokine, or other T cell or NK cell supplement or a combination thereof. In some embodiments, a kit can include one or more reagents for culturing cells disclosed herein. For example, a kit can include media, growth factors, cytokines and/or one or more selection reagent for increasing transfection and construct-containing/construct-expressing cells as disclosed herein. In some embodiments, a kit can further include an insert with instruction to generate a CAR T cell therapy disclosed herein according to the methods disclosed herein.

In other embodiments, kits are provided for use in treating or alleviating a targeted disease or condition treatable by use of a CAR T cell therapy disclosed herein such as cancer and/or a tumor or other malignancy as described herein. In some embodiments, the kit can include instructions for use in accordance with any of the methods described herein. The included instructions can include a description of administration of any the polynucleotides, vectors expressing constructs disclosed herein, cells and/or pharmaceutical compositions (e.g., CAR T cell therapy) described herein and optionally, radiation therapy to treat, delay the onset, or alleviate a target disease as those described herein. The kit can further include a description of selecting an individual suitable for treatment based on identifying whether that individual has the target disease, e.g., applying the diagnostic method as described herein. In yet other embodiments, the instructions can include a description of administering a CAR T cell therapy to a subject at risk of the target disease.

In certain embodiments, kits include instructions for using the components of the kit, for example relating to the use of a CAR T cell therapy generally include information as to dosage, dosing schedule, and route of administration for the intended treatment. The containers can be unit doses, bulk packages (e.g., multi-dose packages) or sub-unit doses. Instructions supplied in the kits of the invention can be written instructions on a label or package insert (e.g., a paper sheet included in the kit), but machine-readable instructions (e.g., instructions carried on a magnetic or optical storage disk) are also acceptable.

The label or package insert indicates that the composition is used for treating, delaying the onset and/or alleviating the disease, such as cancer or a tumor (e.g., a sarcoma) or other malignancy. Instructions can be provided for practicing any of the methods described herein.

Kits disclosed herein include suitable packaging. Suitable packaging includes, but is not limited to, vials, bottles, microfuge or other tubes, plates, jars, flexible packaging (e.g., sealed Mylar or plastic bags), and the like. Also contemplated are packages for use in combination with a specific device, such as an inhaler, nasal administration device (e.g., an atomizer) or an infusion device such as a minipump, a syringe for portal vein introduction or bolus introduction. A kit can have a sterile access port (for example the container can be an intravenous solution bag or a vial having a stopper pierceable by a hypodermic injection needle). The container can also have a sterile access port (for example the container can be an intravenous solution bag or a vial having a stopper pierceable by a hypodermic injection needle). At least one active agent in the composition can be a CAR T cell therapy as those described herein.

Kits can optionally provide additional components such as buffers and interpretive information. Normally, the kit includes a container and a label or package insert(s) on or associated with the container. In some embodiments, the invention provides articles of manufacture including contents of the kits described above.

### EMBODIMENTS

In embodiment 1 provided herein is a polynucleotide construct encoding at least one chimeric antigen receptor (CAR) and at least one chemokine receptor, wherein the CAR comprises a single-chain variable fragment (scFv) able to bind to B7H3 (B7 Homolog 3, CD276). In embodiment 2 provided herein is the polynucleotide construct according to embodiment 1, wherein the CAR polynucleotide comprises at least 85% identity up to 100% identity to the polynucleotide represented by SEQ ID NO: 1. In embodiment 3 provided herein is the polynucleotide construct according to any one of embodiments 1 or 2, wherein the polynucleotide encoding the chemokine receptor comprises a polynucleotide encoding at least one of C-X-C chemokine receptor 1 (CXCR1), C-X-C chemokine receptor 2 (CXCR2), C-X-C chemokine receptor 3 (CXCR3), C-X-C chemokine receptor 4 (CXCR4), C-X-C chemokine receptor 5 (CXCR5), C-X-C chemokine receptor 6 (CXCR6), C-C chemokine receptor 1 (CCR1), C-C chemokine receptor 2 (CCR2), C-C chemokine receptor 3 (CCR3), C-C chemokine receptor 4 (CCR4), C-C chemokine receptor 5 (CCR5), C-C chemokine receptor 6 (CCR6), C-C chemokine receptor 7 (CCR7), C-C chemokine receptor 8 (CCR8), C-C chemokine receptor 9 (CCR9), C-C chemokine receptor 10 (CCR10), C-C chemokine receptor 11 (CCR11), C chemokine receptor (XCR1), CX3C chemokine receptor (CX3CR1), midkine receptor, or a combination thereof or biologically active fragment thereof. In embodiment 4 provided herein is the polynucleotide construct according to any one of embodiment 1 or 2, wherein the polynucleotide encoding the chemokine receptor comprises a polynucleotide comprising CXCR2 or a biologically active fragment thereof. In embodiment 5 provided herein is the polynucleotide construct according to embodiment 4, wherein the polynucleotide encoding CXCR2 comprises at least 85% identity up to 100% identity to the polynucleotide represented by SEQ ID NO: 2. In embodiment 6 provided herein is the polynucleotide construct according to any one of embodiments 1 through 5, wherein the polynucleotide construct encodes a fusion protein comprising a CAR and a chemokine receptor linked via a polypeptide linker. In embodiment 7 provided herein is the polynucleotide construct according to embodiment 6, wherein the polypeptide linker comprises a self-cleaving polypeptide. In embodiment 8 provided herein is the polynucleotide construct according to embodiment 7, wherein the self-cleaving polypeptide is a 2A peptide.

In embodiment 9 provided herein is a cell comprising a polynucleotide construct according to any one of embodiments 1 through 8.

In embodiment 10 provided herein are one or more polypeptides encoded by a polynucleotide construct according to any one of embodiments 1 through 8.

In embodiment 11 provided herein is a cell comprising one or more polypeptides of embodiment 10.

In embodiment 12 provided herein is a vector comprising a polynucleotide according to any one of embodiments 1 through 8. In embodiment 13 provided herein is the vector according to embodiment 12, wherein the vector is a viral vector, or baboon pseudotyped vector. In embodiment 14 provided herein is the vector according to any one of embodiments 12 through 13, wherein the vector is a viral vector, and the viral vector is a lentiviral, an adenoviral adeno-associated viral, a herpesviral, or a retroviral vector.

In embodiment 15 provided herein is a cell comprising the vector according to any one of embodiments 12 through 14.

In embodiment 16 provided herein is a composition comprising: (a) a first polypeptide comprising a CAR; and (b) a second polypeptide comprising a chemokine receptor. In embodiment 17 provided herein is the composition of embodiment 16, wherein the first and second polypeptides are linked by a polypeptide linker. In embodiment 18 provided herein is the composition of embodiment 17, wherein the polypeptide peptide linker comprises a cleavage site. In embodiment 19 provided herein is the composition of embodiment 18, wherein the cleavage site comprises a self-cleaving polypeptide. In embodiment 20 provided herein is the composition of embodiment 19, wherein the self-cleaving polypeptide comprises a 2A peptide. In embodiment 21 provided herein is the composition of any one of embodiments 16 through 20, wherein the first and the second polypeptides are separate polypeptides. In embodiment 22 provided herein is the composition of any one of embodiments 16 through 21, wherein the first polypeptide comprising a CAR comprises a single-chain variable fragment (scFv) able to bind to B7H3 (B7 Homolog 3, CD276). In embodiment 23 provided herein is the composition of embodiment 22, wherein the first polypeptide comprising a CAR comprises a sequence at least 85% identical up to 100% identical to a polypeptide encoded by SEQ ID NO: 1. In embodiment 24 provided herein is the composition of any one of embodiments 16 through 23, wherein the second polypeptide comprising a chemokine receptor comprises a Cys-Cys motif receptor (CCR) or a Cys-X-Cys motif receptor (CXCR). In embodiment 25 provided herein is the composition of embodiment 24, wherein the second polypeptide comprising a chemokine receptor comprises a C-X-C chemokine receptor 1 (CXCR1), C-X-C chemokine receptor 2 (CXCR2), C-X-C chemokine receptor 3 (CXCR3), C-X-C chemokine receptor 4 (CXCR4), C-X-C chemokine receptor 5 (CXCR5), C-X-C chemokine receptor 6 (CXCR6), C-C chemokine receptor 1 (CCR1), C-C chemokine receptor 2 (CCR2), C-C chemokine receptor 3 (CCR3), C-C chemokine receptor 4 (CCR4), C-C chemokine receptor 5 (CCR5), C-C chemokine receptor 6 (CCR6), C-C chemokine receptor 7 (CCR7), C-C chemokine receptor 8 (CCR8), C-C chemokine receptor 9 (CCR9), C-C chemokine receptor 10 (CCR10), C-C chemokine receptor 11 (CCR11), C chemokine receptor (XCR1), CX3C chemokine receptor (CX3CR1), midkine receptor, or a combination thereof or biologically active fragment thereof. In embodiment 26 provided herein is the composition of embodiment 25, wherein the second polypeptide comprising a chemokine receptor comprises CXCR1 or a CXCR2. In embodiment 27 provided herein is the composition of embodiment 26, wherein the second polypeptide comprising a chemokine receptor comprises CXCR1. In embodiment 28 provided herein is the composition of embodiment 27, wherein the second polypeptide comprising a chemokine receptor is at least 85% up to 100% identical to a polypeptide encoded by SEQ ID NO: 11. In embodiment 29 provided herein is the composition of embodiment 26, wherein the second polypeptide comprising a chemokine receptor comprises CXCR2. In embodiment 30 provided herein is the composition of embodiment 29, wherein the second polypeptide comprising a chemokine receptor is at least 85% up to 100% identical to a polypeptide encoded by SEQ ID NO: 2.

In embodiment 31 provided herein are one or more polynucleotides encoding a composition of any one of embodiments 16 through 30.

In embodiment 32 provided herein is a cell comprising one or more polypeptides of anyone of embodiments 16 through 30 and/or one or more polynucleotide of embodiment 31.

In embodiment 33 provided herein is the cell according to any one of embodiments 9, 11, 15, or 32, wherein the cell comprises a human immune cell or a human stem cell. In embodiment 34 provided herein is the cell according to embodiment 33, wherein the human immune cell comprises a neutrophil, eosinophil, basophil, mast cell, monocyte, macrophage, dendritic cell, natural killer cell, or a lymphocyte. In embodiment 35 provided herein is the cell according to embodiment 34, wherein the human immune cell comprises a natural killer (NK) cell. In embodiment 36 provided herein is the cell according to embodiment 34, wherein the human immune cell comprises a T cell. In embodiment 37 provided herein is the cell according to embodiment 36, wherein the T cell comprises a CD8+ T cell, a CD4+ T cell, a CD8+/CD4+ double positive T cell, or a combination thereof. In embodiment 38 provided herein is the cell according to embodiment 37, wherein the CD8+ T cell comprises a naive CD8+ T cell, a central memory CD8+ T cell, an effector memory CD8+ T cell, a bulk CD8+ T cell, or a combination thereof. In embodiment 39 provided herein is the cell according to embodiment 37, wherein the CD4+ T cell comprises a naive CD4+ T cell, a central memory CD4+ T cell, an effector memory CD4+ T cell, a bulk CD4+ T cell, or a combination thereof. In embodiment 40 provided herein is the cell according to embodiment 36, wherein the T cell comprises a precursor T cell. In embodiment 41 provided herein is the cell according to embodiment 33, wherein the human stem cell comprises a human pluripotent, multipotent stem cell, embryonic stem cell, induced pluripotent stem cell, hematopoietic stem cell, CD34+ cell. In embodiment 42 provided herein is the cell according to embodiment 41, wherein the human stem cell comprises a hematopoietic stem cell. In embodiment 43 provided herein is the cell according to embodiment 41, wherein the human stem cell comprises an induced pluripotent stem cell. In embodiment 44 provided herein is the cell according to any one of embodiments 32 through 43, wherein the cell comprises at least 1.2, 1.4, 1.6, 1.8, 2, 4, 5, 6, 7, 8, or 9-fold and/or no more than 10, 9, 8, 7, 6, 5, 4, 2, 1.8, 1.6, or 1.4-fold increased mitochondrial mass as compared to a corresponding cell lacking a chemokine receptor. In embodiment 45 provided herein is the cell according to embodiment 44, wherein the cell comprises at least 2-fold increased mitochondrial mass as compared to a corresponding cell lacking a chemokine receptor. In embodiment 46 provided herein is the cell according to any one of embodiments 32 through 45, wherein the cell comprises at least 1.2, 1.4, 1.6, 1.8, 2, 4, 5, 6, 7, 8, or 9-fold and/or no more than 10, 9, 8, 7, 6, 5, 4, 2, 1.8, 1.6, or 1.4-fold increased ATP production as compared to a corresponding cell lacking a chemokine receptor. In embodiment 47 provided herein is the cell according to embodiment 46, wherein the cell comprises at least 2-fold increased ATP production as compared to a corresponding cell lacking a chemokine receptor.

In embodiment 48 provided herein is an engineered T cell comprising: (a) a first polynucleotide encoding a first polypeptide comprising a CAR; and (b) a second polynucleotide encoding a second polypeptide comprising a chemokine receptor. In embodiment 49 provided herein is the engineered T cell of embodiment 48, wherein the second polypeptide comprising a chemokine receptor comprises CXCR1 or CXCR2. In embodiment 50 provided herein is the engineered T cell of embodiment 49, wherein the second polypeptide comprising a chemokine receptor comprises CXCR1. In embodiment 51 provided herein is the engineered T cell of embodiment 49, wherein the second polypeptide comprising a chemokine receptor comprises CXCR2. In embodiment 52 provided herein is the engineered T cell of any one of embodiments 48 through 51, further comprising a third polynucleotide encoding a third polypeptide comprising CXCR1 or CXCR2, wherein the third polynucleotide encodes a chemokine receptor different from the chemokine receptor encoded by second polynucleotide. In embodiment 53 provided herein is the engineered T cell of any one of embodiments 48 through 52, wherein the CAR comprises a single-chain variable fragment (scFv) able to bind to B7H3 (B7 Homolog 3, CD276). In embodiment 54 provided herein is the engineered T cell of embodiment 53, wherein the first polynucleotide comprises a sequence at least 85% identical up to 100% identical to SEQ ID NO: 1. In embodiment 55 provided herein is the engineered T cell of any one of embodiments 48 through 54, wherein the second or third polynucleotide is at least 85% up to 100% identical to SEQ ID NO: 11. In embodiment 56 provided herein is the engineered T cell of any one of embodiments 48 through 54, wherein the second or third polynucleotide is at least 85% up to 100% identical to SEQ ID NO: 2. In embodiment 57 provided herein is the engineered T cell of any one of embodiments 48 through 54, wherein the second polynucleotide is at least 85% up to 100% identical to SEQ ID NO: 2 and the third polynucleotide is at least 85% up to 100% identical to SEQ ID NO: 11. In embodiment 58 provided herein is the engineered T cell of any one of embodiments 48 through 57, wherein the engineered T cell comprises a CD8+ T cell, a CD4+ T cell, a CD8+/CD4+ double positive T cell, or a combination thereof. In embodiment 59 provided herein is the engineered T cell of embodiment 58, wherein the CD8+ T cell comprises a naive CD8+ T cell, a central memory CD8+ T cell, an effector memory CD8+ T cell, a bulk CD8+ T cell, or a combination thereof. In embodiment 60 provided herein is the engineered T cell of embodiment 58, wherein the CD4+ T cell comprises a naive CD4+ T cell, a central memory CD4+ T cell, an effector memory CD4+ T cell, a bulk CD4+ T cell, or a combination thereof. In embodiment 61 provided herein is the engineered T cell of any one of embodiments 48 through 57, wherein the engineered T cell comprises a precursor T cell.

In embodiment 62 provided herein is a pharmaceutical composition comprising a cell of any one of embodiments 9, 11, 15, or 32 through 61 and a pharmaceutically acceptable excipient or carrier.

In embodiment 63 provided herein is a method of generating a cell population expressing a polypeptide according to embodiment 10 comprising: (a) introducing the vector according to any one of embodiments 12 through 14 or a transposon including a polynucleotide according to any one of embodiments 1 to 5 into a lymphocyte population or editing a lymphocyte population to express a polypeptide according to embodiment 10; (b) culturing the lymphocyte population in media and one or more of an anti-CD3 antibody, an anti-CD28 antibody, a cytokine; and (c) enriching for the lymphocyte population expressing the polypeptide according to embodiment 10. In embodiment 64 provided herein is the method according to embodiment 63, wherein enriching for the lymphocyte population comprises contacting the lymphocyte population comprising the vector according to any one of embodiments 6-8 with a selection reagent. In embodiment 65 provided herein is the method according to embodiment 64, wherein the selection reagent comprises methotrexate or other suitable selection agent. In embodiment 66 provided herein is the method according to any one of embodiments 63 through 65, wherein the cytokine comprises one or more of interleukin-2 (IL-2), interleukin-7 (IL-17), interleukin-15 (IL-15), interleukin-7 (IL-7), interleukin-12 (IL-12), interleukin-18 (IL-18), interleukin-21 (IL-21), or a combination thereof. In embodiment 67 provided herein is the method according to any one of embodiments 63 through 66, wherein the lymphocyte population comprises comprise CD8+ T cell, a CD4+ T cell, a CD8+/CD4+ double positive T cell, or a combination thereof. In embodiment 68 provided herein is the method according to embodiment 63, wherein the lymphocyte population is further exposed to interleukin-8 (IL-8) to induce caspase production in the lymphocyte population.

In embodiment 69 provided herein is a method of killing a target cell comprising surface expressed B7H3, the method comprising contacting the target cell with a cell of any one of embodiments 9, 11, 15, or 32 through 61 or a pharmaceutical composition of embodiment 62. In embodiment 70 provided herein is the method of embodiment 69, wherein the target cell secretes a cytokine. In embodiment 71 provided herein is the method of embodiment 70, wherein the cytokine comprises a chemokine. In embodiment 72 provided herein is the method of embodiment 71, wherein the chemokine comprises an interleukin. In embodiment 73 provided herein is the method of embodiment 72, wherein the interleukin comprises IL-1, 2, 3, 5, 6, 7, 8, or a combination thereof. In embodiment 74 provided herein is the method of embodiment 73, wherein the interleukin comprises IL-6, IL-8, or a combination thereof. In embodiment 75 provided herein is the method of embodiment 74, wherein the interleukin comprises IL-8.

In embodiment 76 provided herein is a method of preventing development of, treating, or ameliorating a cancer in a subject, the method comprising administering the pharmaceutical composition of embodiment 62. In embodiment 77 provided herein is the method according to embodiment 76, wherein the cancer comprises a tumor or a malignancy. In embodiment 78 provided herein is the method according to any one of embodiments 76 or 77, further comprising irradiating the tumor prior to, simultaneously, or after administering the composition to the subject. In embodiment 79 provided herein is the method according to embodiment 78, wherein irradiating the tumor comprises administering at least one dose of radiation ranging from about 2 Gy to about 50 Gy. In embodiment 80 provided herein is the method according to any one of embodiments 76 through 79, wherein the tumor comprises at least one B7H3+ cell, at least one IL-8+ cell, or a combination thereof. In embodiment 81 provided herein is the method according to any one of embodiments 76 through 80, wherein the administration is intravenously, by bolus, intrarenally, topically or other suitable mode. In embodiment 82 provided herein is the method according to any one of embodiments 76 through 80, wherein the administration is locally to the tumor in the subject. In embodiment 83 provided herein is the method according to any one of embodiments 76 through 82, wherein the tumor comprises a solid tumor. In embodiment 84 provided herein is the method according to any one of embodiments 76 through 82, wherein the tumor comprises breast, lung, brain, head and neck, prostate, esophageal, stomach or other gastrointestinal tumor, colon, liver, kidney, eye, skin, or other tumor or blood malignancy or other solid tumor or other malignancy. In embodiment 85 provided herein is the method according to any one of embodiments 76 through 83, wherein the tumor comprises a sarcoma. In embodiment 86 provided herein is the method according to embodiment 85, wherein the sarcoma comprises a bone sarcoma, a soft-tissue sarcoma, or other sarcoma or a combination thereof. In embodiment 87 provided herein is the method according to embodiment 86, wherein the bone sarcoma comprises one or more of osteosarcoma, chondrosarcoma, poorly differentiated round/spindle cell tumors, Ewing sarcoma, hemangioendothelioma, angiosarcoma, fibrosarcoma/myofibrosarcoma, chordoma, adamantinoma, liposarcoma, leiomyosarcoma, malignant peripheral nerve sheath tumor, rhabdomyosarcoma, synovial sarcoma, malignant solitary fibrous tumor, or other bone sarcoma. In embodiment 88 provided herein is the method according to embodiment 86, wherein the soft-tissue sarcoma comprises one or more of liposarcoma, atypical lipomatous tumor, dermatofibrosarcoma protuberans, malignant solitary fibrous tumor, inflammatory myofibroblastic tumor, low-grade myofibroblastic sarcoma, fibrosarcoma, myxofibrosarcoma, low-grade fibromyxoid sarcoma, giant cell tumor of soft tissues, leiomyosarcoma, malignant glomus tumor, rhabdomyosarcoma, hemangioendothelioma, angiosarcoma of soft tissue, extraskeletal osteosarcoma, gastrointestinal stromal tumor, malignant, malignant peripheral nerve sheath tumor, malignant Triton tumor, malignant granular cell tumor, malignant ossifying fibromyxoid tumor, stromal sarcoma not otherwise specified, myoepithelial carcinoma, malignant phosphaturic mesenchymal tumor, synovial sarcoma, epithelioid sarcoma, alveolar soft part sarcoma, clear cell sarcoma of soft tissue, extraskeletal myxoid chondrosarcoma, extraskeletal Ewing sarcoma, desmoplastic small round cell tumor, extrarenal rhabdoid tumor, perivascular epithelioid cell tumor, intimal sarcoma, undifferentiated spindle cell sarcoma, undifferentiated pleomorphic sarcoma, undifferentiated round cell sarcoma, undifferentiated epithelioid sarcoma, undifferentiated sarcoma, not otherwise specified, or other soft tissue sarcoma or a combination thereof.

### EXAMPLES

The following examples are included to illustrate certain embodiments. It should be appreciated by those of skill in the art that the techniques disclosed in the examples which follow represent techniques discovered to function well in the practice of the claimed methods, compositions, and apparatus. However, those of skill in the art should, in light of the present disclosure, appreciate that changes can be made in some embodiments which are disclosed and still obtain a like or similar result without departing from the spirit and scope of the invention.

### Example 1

In one exemplary method, chemokine expression in cancer cells was analyzed in the presence and absence of radiation. In this method, chemokine expression was measured in osteosarcoma and rhabdomyosarcoma cell lines with and without irradiation. Two human osteosarcoma cell lines (HOS and OS-17) and three human rhabdomyosarcoma cell lines (RH30, RD, and JR-1) were grown to confluence and were either remained untreated (control) or irradiated with 50 gray (Gy) radiation, a clinically achievable dose of irradiation. After treatment cell lysates were prepared. For this example, cells were rinsed with PBS before adding lysis buffer. Cells were solubilized at 1×10⁷ cells/mL in lysis buffer. Cells were pipetted up and down to resuspend and the lysates were rocked gently at 2-8° C. for 30 minutes. Cells were then microcentrifuged at 14,000×g for 5 minutes, and the supernatant was transfer into a clean test tube. Quantitation of protein concentration in the cell lysate was determined using a total protein assay.

Then cell lysate samples were run on each human chemokine array. The human chemokine array used in the examples was a membrane-based sandwich immunoassay that simultaneously detected expression of the following human chemokines in a head-to-head manner in duplicate: CL1/I-309; CCL21/6Ckine; CXCL8/IL-8; CCL2/MCP-1; CCL22/MDC; CXCL9/MIG; CCL3/CCL4 (MIP-1 alpha/MIP-1 beta); CCL26/Eotaxin-3; CXCL10/IP-10; CCL5/RANTES; CCL28; CXCL11/I-TAC; CCL7/MCP-3; Chemerin; CXCL12/SDF-1; CCL14/HCC-1/HCC-3; CX3CL1/Fractalkine; CXCL16; CCL15/MIP-1 delta/LKN-1; CXCL1/GRO alpha; CXCL17/VCC-1; CCL17/TARC; CXCL4/PF4; IL-16; CCL18/PARC; CXCL5/ENA-78; Midkine; CCL19/MIP-3 beta; CXCL7/NAP-2; and XCL1/Lymphotactin.

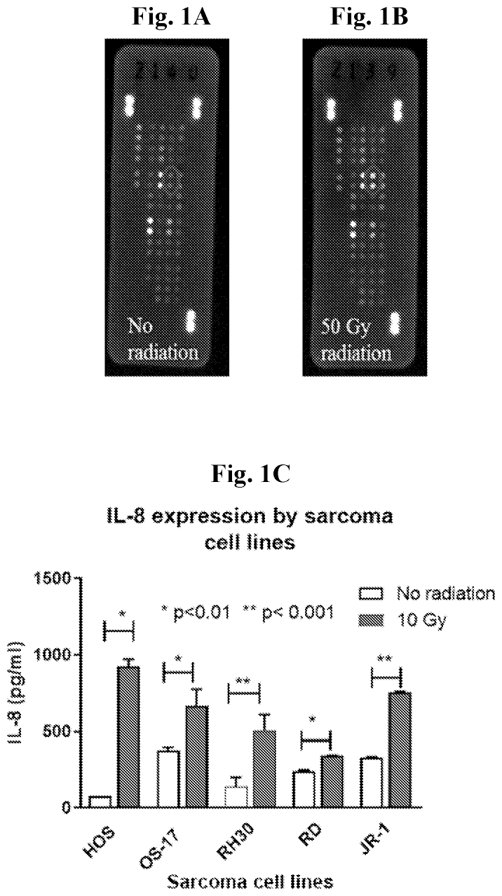

FIGS. 1A and 1B illustrates representative images of chemokine expression in human osteosarcoma cells that were untreated (FIG. 1A) or irritated (FIG. 1B) were each dot represents a different chemokine. Although several chemokines were detected at baseline (untreated cells, FIG. 1A), IL-8 was the only chemokine upregulated by 50 Gy radiation (FIG. 1B). The circled dots in FIGS. 1A and 1B represent IL-8 expression detected on the array.

Two human osteosarcoma cell lines (HOS and OS-17) and three human rhabdomyosarcoma cell lines (RH30, RD, and JR-1) were this time irradiated with 10 Gy radiation prior to harvesting the cells and preparing cell lysate as described above. Cell lysate from irradiated and untreated cells were subjected to ELISA analysis to determine IL-8 expression. FIG. 1C illustrates that IL-8 expression was significantly increased in all irradiated cells compared to untreated cells.

In other methods, an IL-8 knockout RH30 cell line was created using CRISPR-Cas9. Any suitable CRISPR-cas system can be used, such as a Class I or a Class II system, for example a Cas9, Cas12a, Cas12b, Cas12c, Cas12d, or Cas12e.

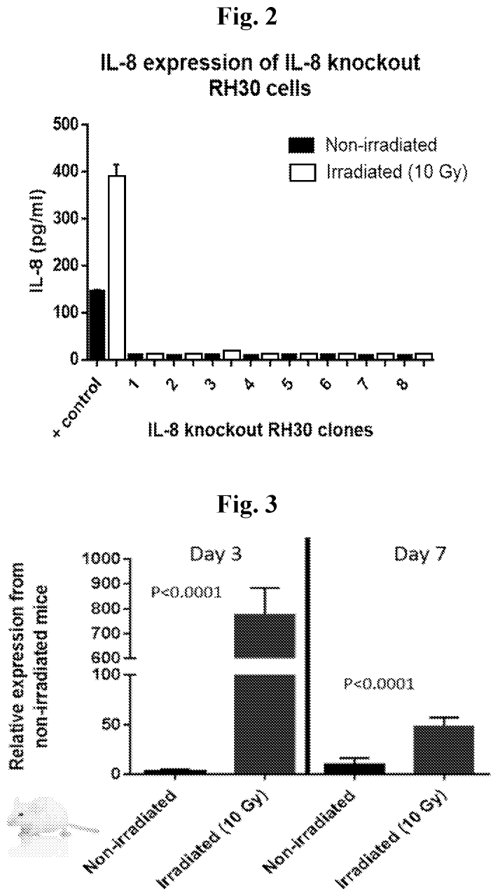

FIG. 2 illustrates a complete knockout of IL-8 expression in eight RH30 clones which is a human abdomyosarcoma cell line. It was observed that all clones of the IL-8 knockout RH30 cell line cells grow similarly to non-edited RH30 cells in vitro and do not increase IL-8 expression in response to radiation (FIG. 2).

Next, patient-derived osteosarcoma xenografts were implanted into the pretibial space of immunodeficient NOD-scid IL2rynull (NSG) mice. The mice carry two mutations on the NOD/ShiLtJ genetic background: severe combined immune deficiency (scid) and a complete null allele of the IL2 receptor common gamma chain (IL2rg^{null}). Nin brief NSG were implanted with osteosarcoma tumor cells (10⁶ cells/mouse) in the lateral thigh muscle. Tumors were permitted to grow to 0.5 cm³ in size, at which time the tumors in half of the mice were irradiated (10 Gy) and the other half served as non-irradiated controls. The tumor tissue was harvested at 3 days and at 7 days following irradiation. The harvested tissue was processed and subjected to qRT-PCR analysis to measure CXCL8 (IL-8) expression in the tumor tissue. As illustrated in FIG. 3, IL-8 expression was not elevated in the tumors of the untreated mice; however, tumors harvested after irradiation had increased IL-8 expression demonstrating the effect of irradiation on IL-8 expression.

The data provided in these exemplary methods demonstrates IL-8 was induced to high levels in OS and RMS tumors after subtherapeutic doses of irradiation in vitro and in vivo.

### Example 2

B7H3 a tumor-associated ligand selectively expressed on multiple tumor types but has limited expression in normal tissues.

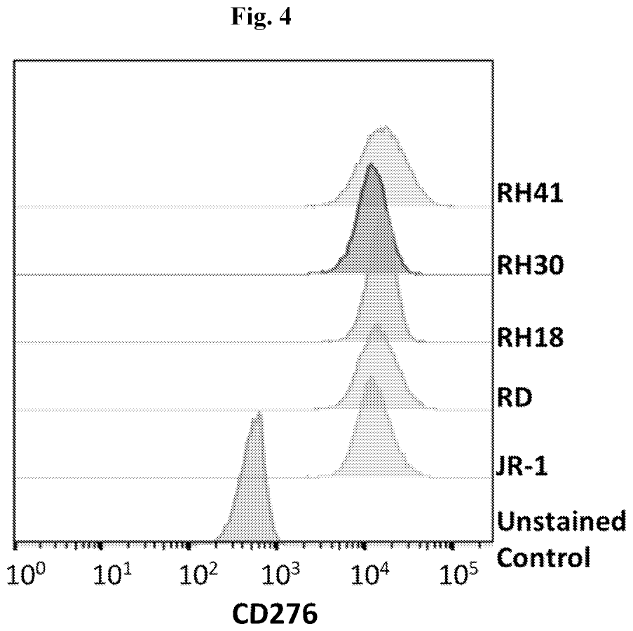

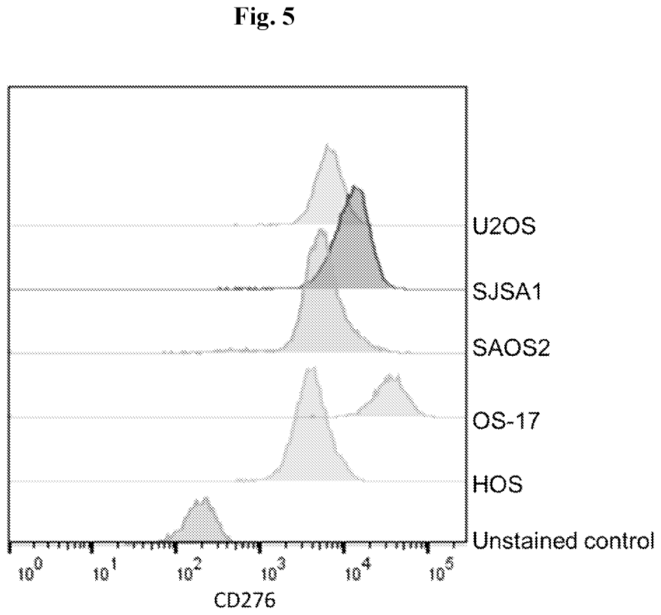

In another exemplary method, to determine if B7H3 (CD276) is uniformly expressed in sarcoma cells, five human rhabdomyosarcoma cell lines (RH41, RH30, RH18, RD, and JR-1) were subjected to flow cytometry and stained for CD276. As illustrated in FIG. 4, B7H3 (CD276) was uniformly expressed on all tested cell lines. The experiment was repeated, this time subjecting a human rhabdomyosarcoma cell line (RMS) and a human osteosarcoma cell line (OS) to flow cytometry and stained for CD276 which is also referred to as B7H3. FIG. 5 illustrates that B7H3 (CD276) was uniformly expressed in both human rhabdomyosarcomas and human osteosarcomas. The data provided in this exemplary method demonstrated that B7H3 was highly expressed by sarcomas.

### Example 3

In another exemplary method, the IL-8 receptor, CXCR2, was cloned into a retroviral vector using gateway cloning techniques. To construct the B7H3-T2A-CXCR2 retroviral transfer vector (B2C), three major steps were performed: 1) cut Her2bbz, a retroviral backbone plasmid, with XhoI and BamHI restriction enzymes; 2) then PCR amplify B7H3 CAR (B7H3 scFv-CD28 TM-41BB-CD3 zeta) using the B7H3 CAR plasmid as the template; and 3) generate a gblock of CXCR2 from Integrated DNA technologies (IDT). Each of these products (vectors and inserts) had an overhang between 20-35 bps with the desired annealing sites. Using NEB HiFi Assembly, the three fragments were then combined in equimolar ratios, incubated for 15 minutes at 50° C., and transformed DH5-alpha E coli.

To PCR amplify B7H3, the following primers were used: B7H3_R_KW: CCCTCTCCACTGCCGCTAGCCCTTGGGGGCAGGGCCTG (SEQ ID NO: 7) and RetroF: CTGCCGACCCCGGGGGTGGACCATCCTCTAGACTGCTCGAGGCCCGCCACCATGCTG CTG (SEQ ID NO: 8). The CXCR2 gblock had the following sequence:

(CXCR2 gblock)
SEQ ID NO: 9
TGCAGGCCCTGCCCCCAAGGGCTAGCGGCAGTGGAGAGGGCAGAGG
AAGTCTGCTAACATGCGGTGACGTCGAGGAGAATCCTGGCCCAAT
GGAAGATTTTAACATGGAGAGTGACAGCTTTGAAGATTTCTGGAA
AGGTGAAGATCTTAGTAATTACAGTTACAGCTCTACCCTGCCCCC
TTTTCTACTAGATGCCGCCCCATGTGAACCAGAATCCCTGGAAAT
CAACAAGTATTTTGTGGTCATTATCTATGCCCTGGTATTCCTGCT
GAGCCTGCTGGGAAACTCCCTCGTGATGCTGGTCATCTTATACAG
CAGGGTCGGCCGCTCCGTCACTGATGTCTACCTGCTGAACCTAGC
CTTGGCCGACCTACTCTTTGCCCTGACCTTGCCCATCTGGGCCGC
CTCCAAGGTGAATGGCTGGATTTTTGGCACATTCCTGTGCAAGGT
GGTCTCACTCCTGAAGGAAGTCAACTTCTATAGTGGCATCCTGCT
ACTGGCCTGCATCAGTGTGGACCGTTACCTGGCCATTGTCCATGC
CACACGCACACTGACCCAGAAGCGCTACTTGGTCAAATTCATATG
TCTCAGCATCTGGGGTCTGTCCTTGCTCCTGGCCCTGCCTGTCTT
ACTTTTCCGAAGGACCGTCTACTCATCCAATGTTAGCCCAGCCTG
CTATGAGGACATGGGCAACAATACAGCAAACTGGCGGATGCTGTT
ACGGATCCTGCCCCAGTCCTTTGGCTTCATCGTGCCACTGCTGAT
CATGCTGTTCTGCTACGGATTCACCCTGCGTACGCTGTTTAAGGC
CCACATGGGGCAGAAGCACCGGGCCATGCGGGTCATCTTTGCTGT
CGTCCTCATCTTCCTGCTCTGCTGGCTGCCCTACAACCTGGTCCT
GCTGGCAGACACCCTCATGAGGACCCAGGTGATCCAGGAGACCTG
TGAGCGCCGCAATCACATCGACCGGGCTCTGGATGCCACCGAGAT
TCTGGGCATCCTTCACAGCTGCCTCAACCCCCTCATCTACGCCTT
CATTGGCCAGAAGTTTCGCCATGGACTCCTCAAGATTCTAGCTAT
ACATGGCTTGATCAGCAAGGACTCCCTGCCCAAAGACAGCAGGCC
TTCCTTTGTTGGCTCTTCTTCAGGGCACACTTCCACTACTCTCTA
AGGATCCGATAAAATAAAAGATTTTATTTAGTCTCCAGAAAAAGG
G

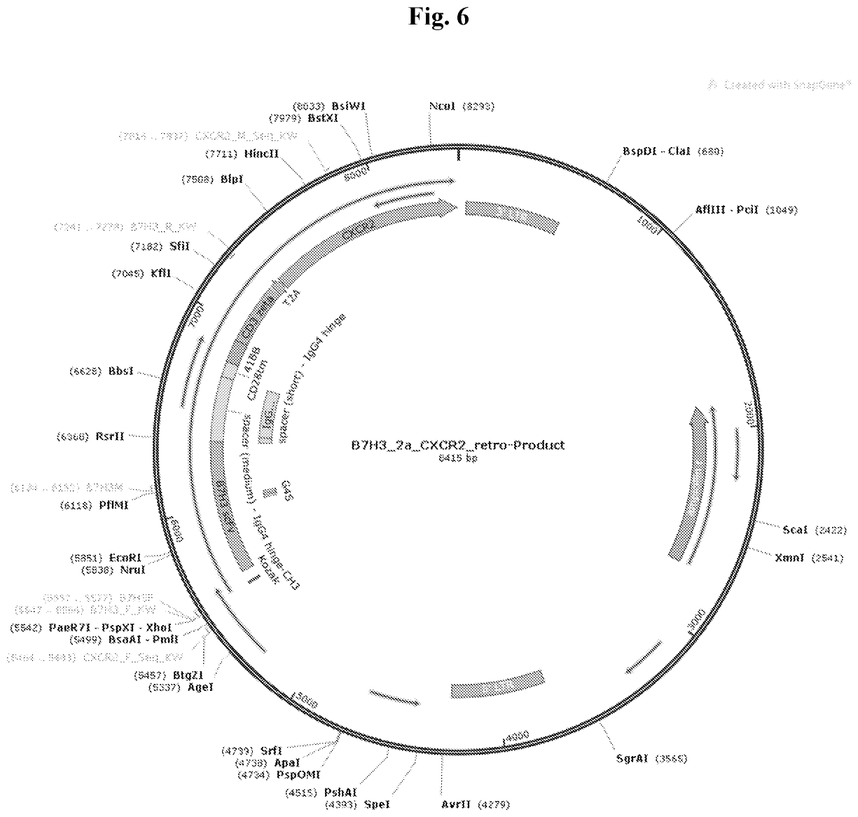

The CXCR2-T2A-B7H3 CAR (as illustrated in FIG. 6) was cloned into lentiviral vectors containing an EF1α promoter using NEBuilder HiFi DNA assembly. The constructed a retroviral transfer plasmid was then transformed into E. coli, selected for the plasmid with carbenicillin, isolated the plasmid DNA from the E. coli cultures. Correct incorporation of IL-8R and CAR sequence were confirmed by Sanger sequencing.

Lentiviral production was done using Lipofectamine® 3000. Using B2C and RD114 (a packaging vector) 293GP cells were then transfected on day 0, refed the virus on day 1, and harvested the viral supernatant on day 2 and day 3. Using this supernatant, T cells were transduced using an optimized protocol.

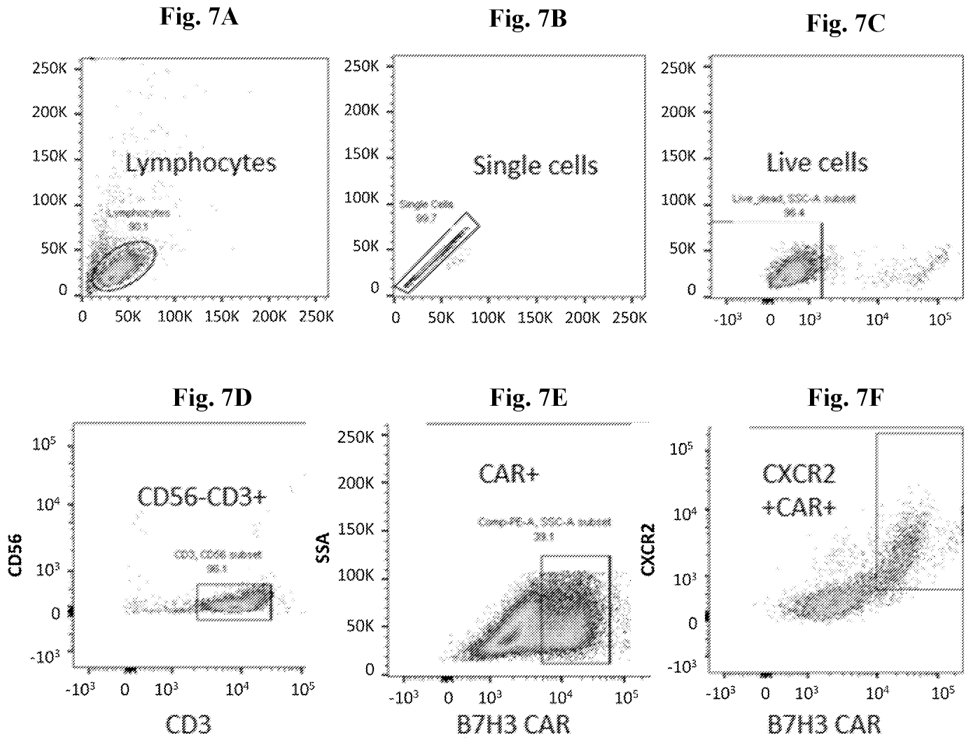

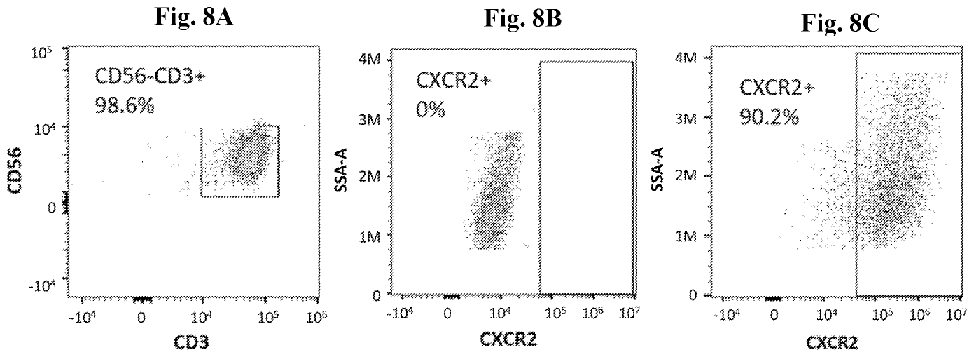

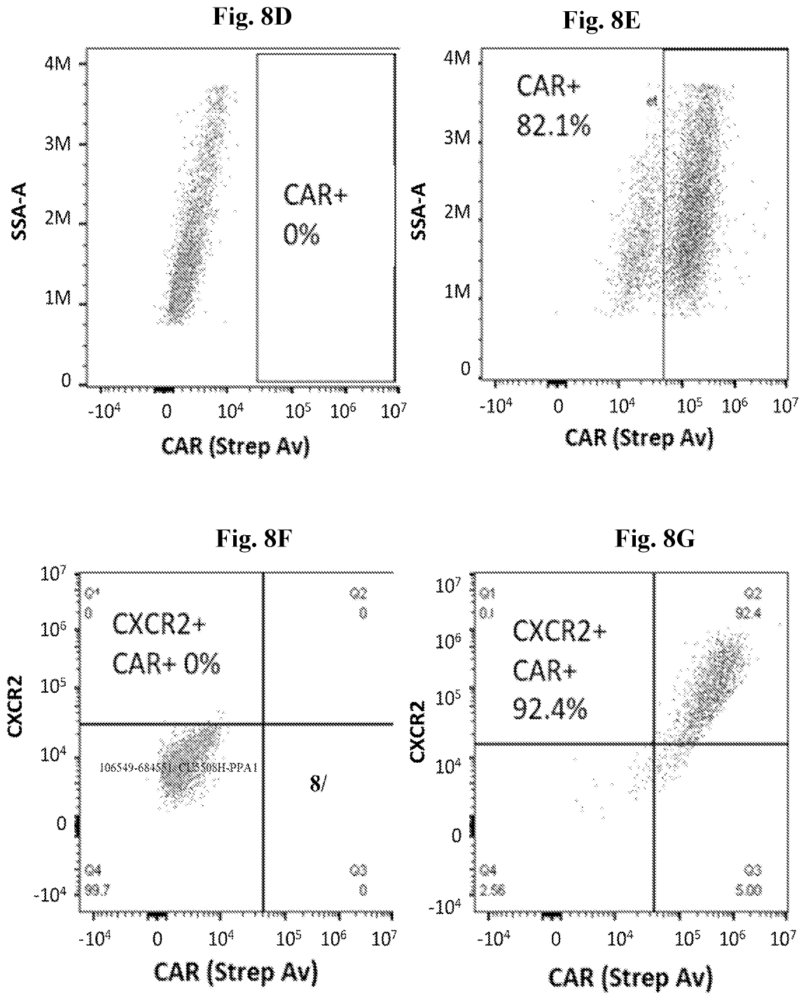

Flow cytometry was performed on the transduced T cells stimulated with CD3/28 beads and IL-2. FIGS. 7A-7F and 8A-8G demonstrated successful transduction of both the CXCR2-T2A-B7H3 CAR and the B7H3 CAR vectors. FIGS. 7A-7F illustrate flow cytometry of T cells gated for lymphocytes (FIG. 7A), single cells (FIG. 7B), live cells (FIG. 7C), cells expressing CD56-CD3+(FIG. 7D), B7H3 CAR+ T cells (FIG. 7E), and CXCR2+B7H3 CAR+ T cells (FIG. 7F). FIGS. 8A-8G illustrate flow cytometry of non-transfected CD56-CD+ T cells (FIG. 8A), non-transfected CXCR2+ T cells (FIG. 8B), CXCR2-transduced CXCR2+ T cells (FIG. 8C), non-transfected CAR+ T cells (FIG. 8D), B7H3 CAR-transduced CAR+ T cells (FIG. 8E), non-transfected CXCR2+CAR+ T cells (FIG. 8F), and dual CXCR2+B7H3 CAR-transduced CXCR2+CAR+ T cells (FIG. 8G).

The data provided in this one exemplary method showed preparation of vectors containing dual CXCR2+B7H3 CAR and that T cells were successfully transduced by retroviruses containing the vectors to product CXCR, B7H3 CAR and dual CXCR2+B7H3 CAR T cells.

In another exemplary method, the IL-8 receptor, CXCR2, is cloned into the “safe harbor” locus AVVS1, which is resistant to gene silencing, using CRISPR/Cas9 gene targeting strategies.

### Example 4

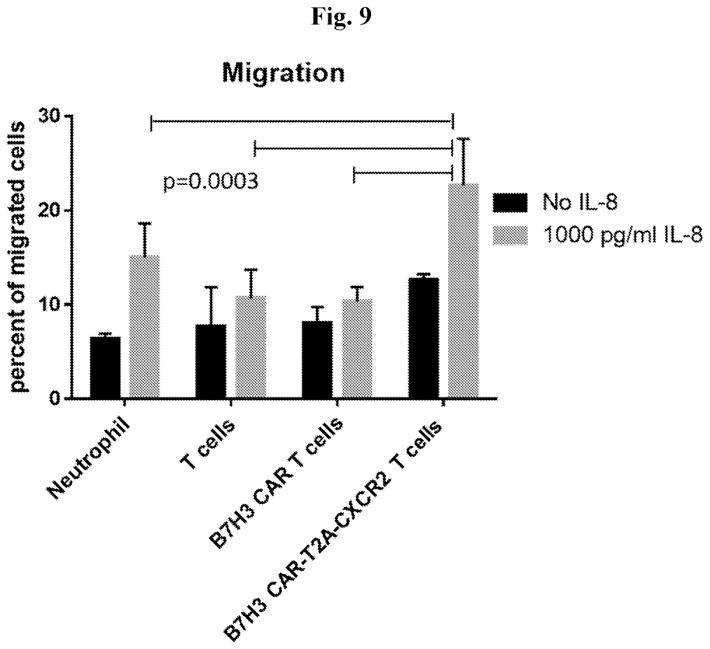

In another exemplary method, effects of the B7H3 CAR T and dual CXCR2+B7H3 CAR T cells were examined. First, a transwell cell migration assay was performed. A transwell cell migration assay measures the chemotactic capability of cells toward a chemo-attractant. FIG. 9 illustrate a transwell migration assay illustrating the increased migration toward an IL-8 gradient of the dual-expressing T cells over B7H3 CAR-expressing T cells and neutrophils, which naturally express both IL-8Rs. These data illustrated that IL-8 receptor+B7H3 CAR-expressing T cells home to an IL-8 gradient in vitro better than B7H3 CAR-expressing T cells.

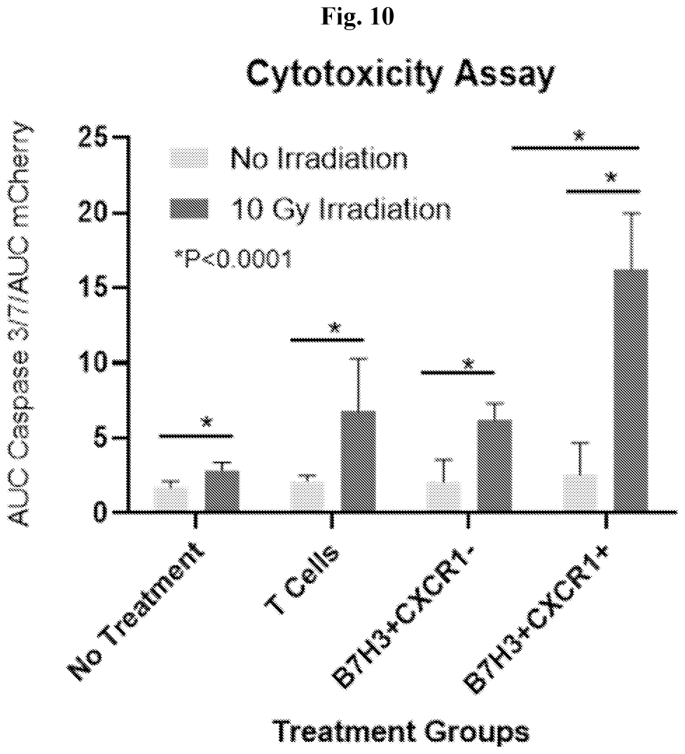

Next, a cytotoxicity was assessed using a cytotoxicity assay (e.g., Incucyte). In brief, sarcoma cells were grown to confluency and were either not irradiated (non-irradiated) or irradiated with 10 Gy radiation. Next, non-irradiated and irradiated cells were treated with either CXCR2+B7H3 CAR T cells or B7H3 CAR (alone) T cells. Incucyte Cytotox Dyes were then added to the culture medium. Incucyte Cytotox Dyes are inert, non-fluorescent and do not enter viable cells. As the cells died and membrane integrity was lost, the Cytotox Dye entered the cell and fluorescently labeled the nuclei. Dying cells were identified and quantified over time by the appearance of green- (or red) labelled nuclei. As illustrated in FIG. 10, CXCR+B7H3 CAR-expressing T cells killed irradiated sarcoma cells better than B7H3 CAR-expressing T cells.

The data provided in this exemplary method demonstrated that CXCR+B7H3 CAR-expressing T cells homed to the tumor and achieved killing of sarcoma tumors with forced expression of the IL-8R via irradiation and did so better that T cells only expressing B7H3.

It is noted that T cells expressing transfected CXCR (e.g., CXCR2) demonstrated improved function and metabolism of use in tumor microenvironments to enhance tumor killing (data not shown).

### Example 5

In another exemplary method, it is determined how modification of the T cells alters their expansion, survival, or cytotoxic abilities. IL-8R signaling in neutrophils is known to not only affect homing but also mediate activation and survival. As such, B7H3 CAR-expressing T cells and CXCR+B7H3 CAR-expressing T cells were subjected to cell counting/membrane-dye dilution assays to measure proliferation. Cytokine production (interferon-γ, TNF, granzyme B, perform) is measured in the B7H3 CAR-expressing T cells and CXCR+B7H3 CAR-expressing T cells by flow cytometry.

### Example 6

In another exemplary method, homing of CXCR+B7H3 CAR-expressing T cells to a sarcoma and killing tumor cells was assessed in vivo. To determine if IL-8 is an important driver behind homing of T cells toward the tumors, NOD-scidIL2rynull (NSG) mice were implanted with luciferase positive RMS tumor cells (10⁶ cells/mouse) in the lateral thigh muscle. Tumors were grown to 0.5 cm³ in size, at which time the tumors in half of the mice are irradiated (10 Gy), and the other half are non-irradiated controls. Three to five days after irradiation mice were injected (via the tail vein, 5×10⁶ cells/mouse) with IL-8R-expressing T cells versus GFP-expressing T cells. Three days after injection, the mice were sacrificed. Tumors were excised, and a portion of the tumor was formalin fixed and paraffin embedded, and the rest rendered to a single cell suspension (using RPMI, collagenase, FBS, and DNase). Tumor samples were assessed for infiltration of T cells based on expression of human CD45, CD3, CD4, CD8, GFP and the presence of IL-8R (CXCR1, CXCR2) by flow cytometry and immunohistochemistry. Mice that receive no T cells are used to provide clean gating for flow cytometry when comparing other groups receiving chemokine receptor-transduced T cells (given that NSG mice do not have native T cells).

To assess ability of CXCR+B7H3 CAR-expressing T cells to kill tumor cells in vivo, NSG mice were implanted with luciferase positive RMS tumor cells (RH30) and irradiated as described above. Three to five days after irradiation, mice were injected (via the tail vein, 5×10⁶ cells/mouse) with either GFP-expressing T cells, B7H3 CAR-expressing T cells, or CXCR+B7H3 CAR-expressing T cells. Mice then underwent tumor volume measurement and bioluminescent imaging once weekly for 4-8 weeks to evaluate tumor reduction/growth. At the end of the evaluation period or when tumors become larger than 2 cm in diameter, a subset of mice were sacrificed, and tumors were processed as described above. Exhaustion of the transferred T cells is assessed in the various groups by flow cytometry using surface receptor expression (given the limited number of cells in the tumor) of standard markers (PD1, TIM3, LAG3, TOX1, TOX2). The remaining subset of mice were used for growth curve analysis to assess for changes in long-term survival.

### Example 7

In another exemplary method, chemotactic migration of B7H3 CAR T cells and B7H3-CXCR2 CAR T cells to IL-8 was assessed. In brief, an Incucyte transwell plate was loaded with recombinant IL-8 at 100 ng/ml (in Roswell Park Memorial Institute Medium (RPMI medium) in the bottom wells. The membrane in the Incucyte transwell plate was coated first with Protein G and ICAM (Intercellular Cell Adhesion Molecule) to simulate extracellular matrix. The membrane was then blocked with phospho-buffered saline (PBS)+2% bovine serum albumin. Two thousand B7H3 or B7H3-CXCR2 CAR T cells were loaded to the top wells (resuspended in RPMI) in quadruplicate wells. The transwell plate was then placed in the Incucyte for imaging for 30 hours.

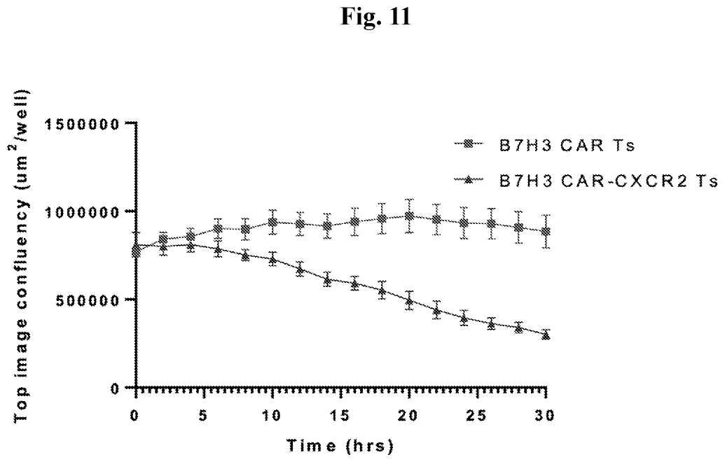

FIG. 11 illustrates that the number of B7H3 CAR T cells and B7H3-CXCR2 CAR T cells that remained in the top chamber of the transwell plate (e.g., the chamber that did not contain recombinant IL-8) over time. The decrease in B7H3-CXCR2 CAR T cells in the top chamber over time reflects that the cells migrated to the recombinant IL-8 in the bottom chamber of the transwell plate. B7H3 CAR T cell numbers in the top chamber remain unchanged over time, demonstrating that, unlike the B7H3-CXCR2 CAR T cells, B7H3 CAR T cells do not migrate to IL-8. These data confirmed that expression of CXCR1/2 (either or both) on the B7H3 CAR T was required for responsiveness to IL-8.

### Example 8

In another exemplary method, interferon-γ (INF-γ) and interleukin-2 (IL-2) levels were assessed in B7H3-CXCR2 CAR T cells and B7H3 CAR T cells co-cultured with irradiated rhabdomyosarcoma. In brief, RH30 (rhabdomyosarcoma) cells were plated in a 96 well plate at 10,000 cells/well in RPMI medium and left overnight in the incubator (˜5% CO₂ at 37° C.) to settle and attach. The following morning (˜12 hours) 10,000 T cells, B7H3 CAR T cells or B7H3-CXCR2 CAR T cells were added to the wells with tumor. All conditions were done in triplicate. After 24 hours, the plate was spun down and supernatant was removed and used for INF-γ and IL-2 ELISA following standard protocols.

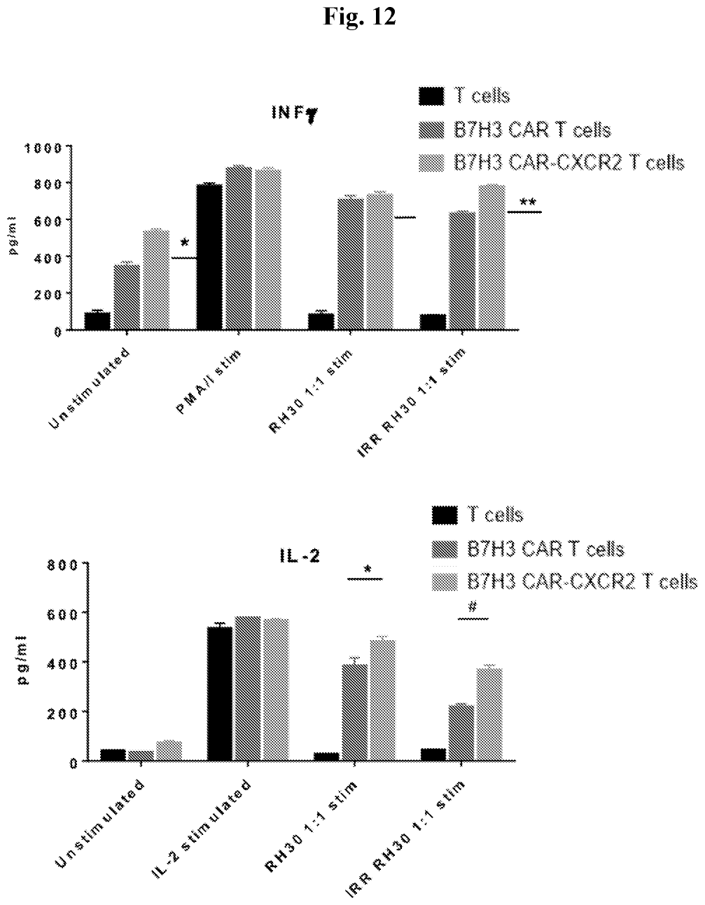

Irradiation leads to IL-8 production in sarcoma cells. INF-γ is critical for the cytotoxic activity of CAR T cells whereas IL-2 promotes T cell expansion. Both INF-γ and IL-2 were significantly increased in the B7H3-CXCR2 CAR T cells that were co-cultured with irradiated rhabdomyosarcoma as compared to the B7H3 CAR T cells co-cultured with irradiated rhabdomyosarcoma (FIG. 12).

As illustrated in FIG. 12, in one set of conditions (labelled IRR RH30 1:1 stim), resting T cells did not respond to irradiated RH30 cells due to a lack of recognition. B7H3 CAR T cells responded to irradiated RH30 cells (due to the CAR) and produced IFN-γ and IL-2. However, B7H3-CXCR2 CAR T cells made significantly more IFN-γ and IL-2 in response to irradiated RH30 cells suggesting that the combination of CAR and CXCR2 signaling was at least additive (and potentially synergistic which was unexpected and surprising).

### Example 9

In another exemplary method, caspase activity—a method of determining killing activity in cells—was assessed in tumor spheroids in the absence or presence of B7H3 CAR T cells or B7H3-CXCR2 CAR T cells. In brief, RH30 cells expressing mCherry were mixed with Poly-vinyl alcohol (PVA) and Matrigel and plated in a low-adherent U bottom plate to form tumor spheroids. The plate was spun down and placed in incubator overnight (˜12 hours). After tumor spheroid formation and incubation, B7H3-CAR T cells or B7H3-CXCR2 CAR T cells were added to the tumor spheroids at a 1:1 ratio and 50 ng/ml of IL-8 was added to each well. Caspase green was then added to the wells. The plate was loaded into the Incucyte machine and images were collected at hour 2 of co-culture and hour 48 of co-culture.

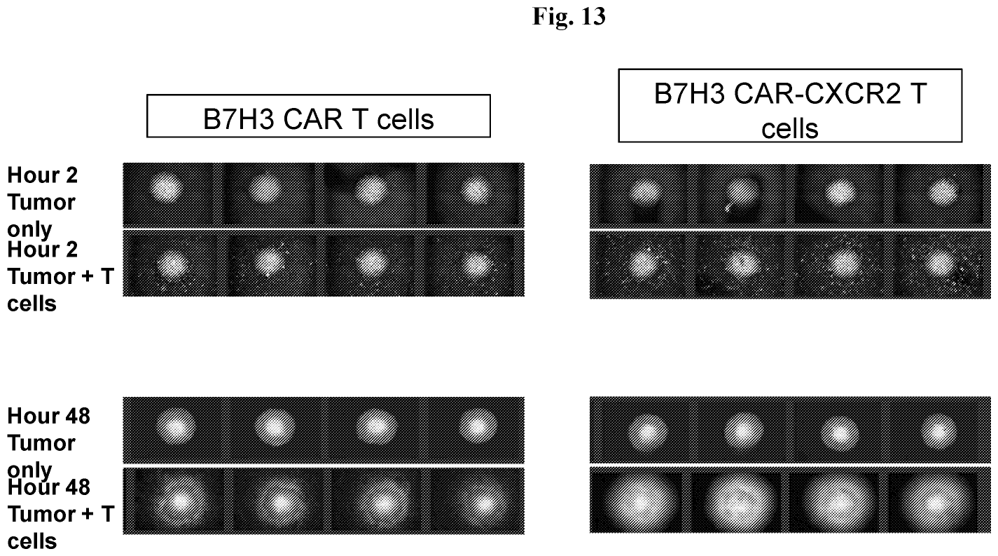

FIG. 13 illustrates that B7H3-CXCR2 CAR T cells in co-culture with RH30 spheroids had greater caspase activity and decreased tumor volume after 48 hours compared to tumor spheroids cultured alone or with B7H3-CAR T cells. The increase in caspase activity after 48 hours demonstrated that B7H3-CXCR2 CAR T cells have a higher cytotoxicity, and therefore, killed more tumor cells than B7H3-CAR T cells alone.

### Example 10

In another exemplary method, caspase activity was assessed over time in tumor spheroids in the presence of B7H3 CAR T cells or B7H3-CXCR2 CAR T cells. In brief, RH30 cells expressing mCherry were mixed with PVA and Matrigel and plated in a low-adherent U bottom plate to form tumor spheroids. The plate was spun down and placed in incubator overnight. After tumor spheroid formation and incubation, B7H3-CAR T cells or B7H3-CXCR2 CAR T cells were added to the tumor spheroids 1:1 and 50 ng/ml of IL-8 was added to each well. Caspase green was then added to the wells. The plate was loaded into the Incucyte machine and images were taken every 2 hours. Within the Incucyte program, a filter was set for minimum fluorescence (green) and caspase activity was quantified by calculating by intensity of fluorescence times the area.

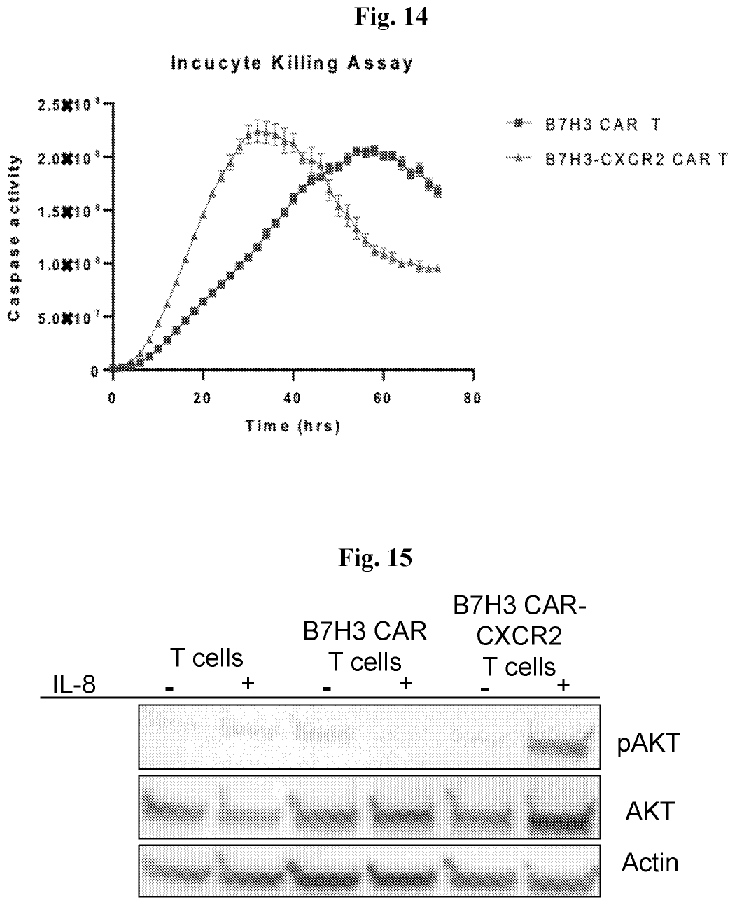

FIG. 14 demonstrates that the B7H3-CXCR2 CAR T cells killed the RH30 tumor earlier, and faster, than the B7H3 CAR T cells alone. FIG. 14 further illustrates that B7H3-CXCR2 CAR T cells killed more tumor cells (e.g., the killing was complete) compared to B7H3 CAR T cells alone.

### Example 11

In another exemplary method, phosphorylation of AKT (Protein kinase B), a downstream target of IL-8 receptor activation, was assessed by Western blot analysis in B7H3 CAR T cells and B7H3-CXCR2 CAR T cells with and without IL-8 stimulation. In brief, T cells, B7H3 CAR T cells, and B7H3-CXCR2 CAR T cells were taken at Day 10 of culture. One million of each were stimulated with 50 ng/ml IL-8 for 10 minutes. After 10 minutes, the stimulated cells as well as 1 million unstimulated of each were pelleted. Protein was extracted using standard techniques. Forty-five nanograms of each sample was lysed in buffer, ran on a 4-20% polyacrylamide gel, and separated by SDS electrophoresis. The gels were then transferred to PVDF membranes and blocked in 5% RPI dry milk powder in a tris buffered saline and 0.1% tween 20 (TBST). After 1 hour, the membranes were left overnight in a 1:1000 dilution of rabbit anti-human primary antibody (pAKT) in 5% BSA in TBST. The following day, the membranes were washed 3 times with TBST and then left in a 1:2000 goat anti-rabbit secondary antibody in 5% milk for 1 hour. Membranes were washed 3 times before being developed using a west fempto maximum sensitivity substrate and imaged (Syngene G: Box). Membrane was stripped following standard Western blot protocols and the process of incubating overnight in primary antibody (AKT, Actin) and additional steps were repeated as detailed above. Actin expression used as a control.

FIG. 15 illustrates that B7H3-CXCR2 CAR T cells stimulated with IL-8 had increased phosphorylation and total AKT protein expression compared to unstimulated B7H3-CXCR2 CAR T cells or both unstimulated and stimulated T cells and B7H3 CAR T cells. AKT is a known pathway in CXCR2 signaling and pAKT is downstream of IL-8 receptor signaling. These data demonstrate a significant increase in pAKT in the B7H3-CXCR2 CAR T cells compared to T cells or B7H3 CAR T cells, the latter of which lack CXCR2 and were incapable of activating IL-8 downstream signaling events.

### Example 12

In another exemplary method, B7H3 CAR T cell and B7H3-CXCR2 CAR T cell effects on tumor cells were assessed in vivo. In brief, NOD-scid IL2rynull (NSG) mice were injected with 250,000 RH30 IL-8 overexpressing tumor cells into their left thigh after cleaning the area with alcohol (Day-3). The mice were imaged three days later and then injected with 3×10⁶ transduced or equivalent number of T cells, B7H3 CAR T cells, or B7H3-CXCR2 CAR T cells. Mice were then imaged weekly until Day 35 and then every 2 weeks. A survival analysis was calculated using GraphPad Prism for each experimental group.

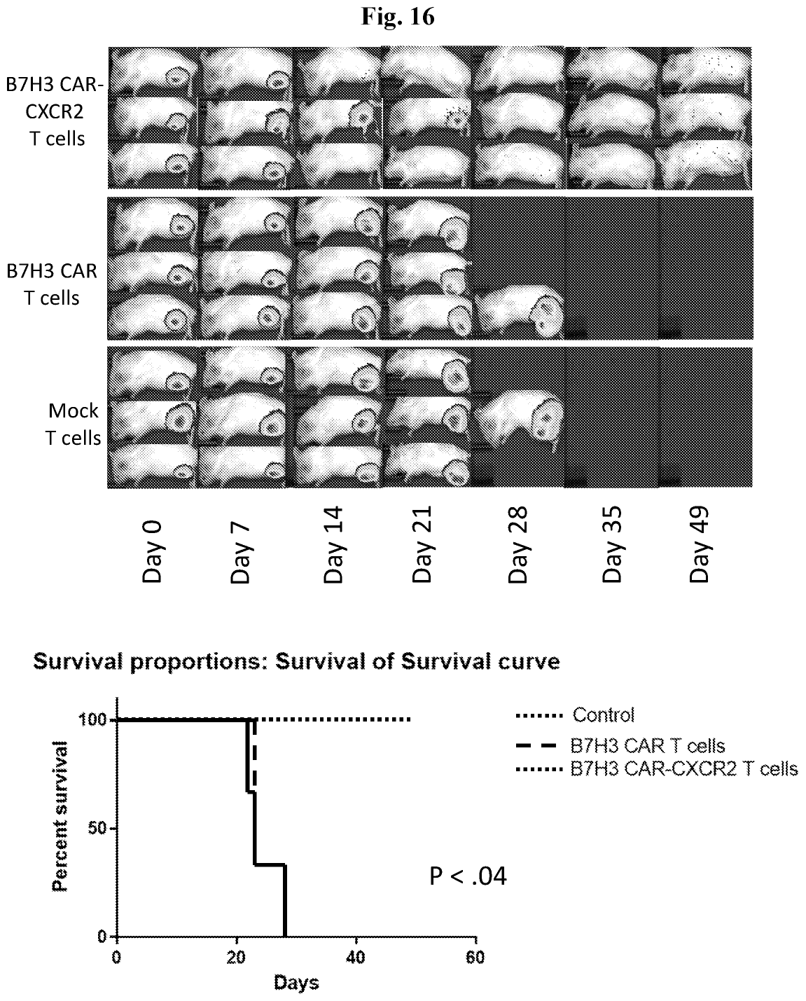

FIG. 16 illustrates bioluminescent imaging of representative mice from each treatment group at day 0, 7, 14, 21, 28, 35, and 49. Tumor volume was reduced in mice injected with B7H3-CXCR2 CAR T cells by about day 14 and there were no tumors detected in mice injected with B7H3-CXCR2 CAR T cells at days 28-49 (FIG. 16). However, mice injected with either T cells or B7H3 CAR T cells did not see improvement in tumor volumes over time and, as reflected in the Kaplan Meier (survival) curve illustrated in FIG. 16, mice from these two treatment groups did not survive past day 35 of the study.

Without being bound by theory, these data suggest that although CAR T cells do not typically home to muscle tissue or solid tumors, B7H3-CXCR2 CAR T cells were more effective at homing to RH30 cells (a cell line of a representative cancer of connective tissue) that expressed IL-8 (e.g., tumor cells) compared to B7H3 CAR T cells not expressing B7H3-CXCR2 (or CXCR1 or both, data not shown).

### Example 13

In another exemplary method, mitochondrial respiration (oxygen consumption) and glycolytic activity (acid production) were assessed in unstimulated and IL-8 stimulated T cells, B7H3 CAR T cells, and B7H3-CXCR2 CAR T cells. In brief, T cells, B7H3 CAR T cells and B7H3-CXCR2 CAR T cells were taken at Day 9 of culture. Half of each were stimulated for 24 hours with 50 ng/ml IL-8. After 24 hours, 1.5 million of each condition were taken for Agilent Seahorse Assay to measure mitochondrial respiration and glycolytic rate following optimized protocols. The Agilent Seahorse Assay measured both the mitochondrial respiration (oxygen consumption) and glycolytic activity (acid production)—both of which are important for the health and adaptability of cells to stressful environments.

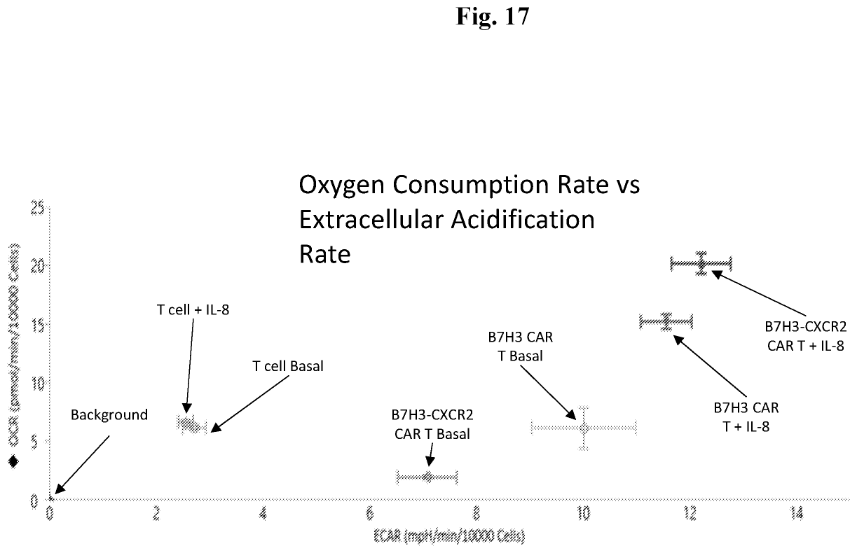

Extracellular oxygen consumption and acid production were greatest in the B7H3-CXCR2 CAR T cells stimulated with IL-8 than the basal (unstimulated) B7H3-CXCR2 CAR T cells or any of the T cell or B7H3 CAR T cell groups (FIG. 17). As both-mitochondrial respiration and glycolytic activity-were increased in the B7H3-CXCR2 CAR T cells compared to T cells and B7H3 CAR T cells, the data showed that these cells had a greater metabolic flexibility to maintain their cytotoxic function in inhospitable environments such as a tumor microenvironment and/or environments of high IL-8 activation.

### Example 14

In another exemplary method, mitochondrial respiration (oxygen consumption) and glycolytic activity (acid production), ATP production, and mitochondrial mass were assessed in unstimulated and IL-8 stimulated B7H3 CAR T cells and B7H3-CXCR2 CAR T cells.

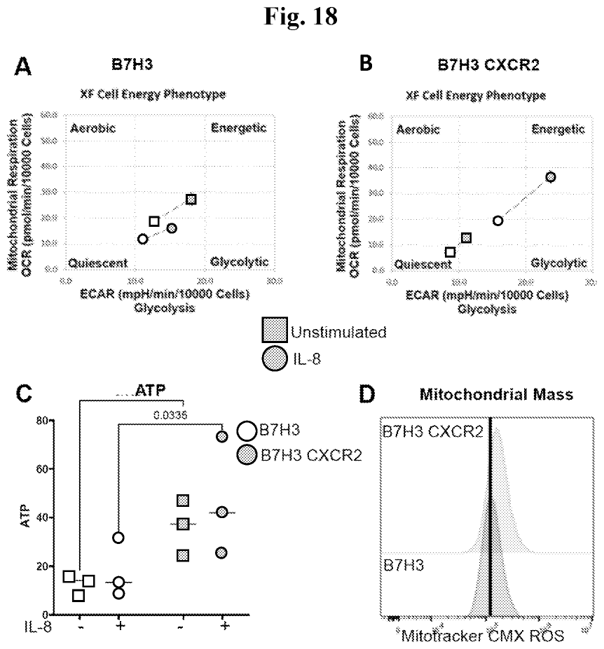

In brief, B7H3 CAR T cells and B7H3-CXCR2 CAR T cells were taken at Day 9 of culture. Half of each were stimulated for 24 hours with 50 ng/ml IL-8. After 24 hours, 1.5 million of each condition were taken for Agilent Seahorse Assay (described above). IL-8 signaling upregulated oxygen consumption (OCR) and extracellular acidification rate (ECAR), which drove B7H3-CXCR2 CARs into a highly energetic state (FIG. 18B). This was not seen in B7H3 alone CAR T cells (FIG. 18A), suggesting that IL-8 impacted both glycolysis and mitochondrial metabolism. Even in the absence of IL-8 stimulation, B7H3-CXCR2 CAR T cells showed higher ATP production (FIG. 18C) and more mitochondria (FIG. 18D) than B7H3 CAR T cells alone. The latter data implies that the B7H3-CXCR2 CAR T cells are metabolically primed to activate OXPHOS by regulating mitochondrial abundance and/or quality prior to IL-8 exposure.

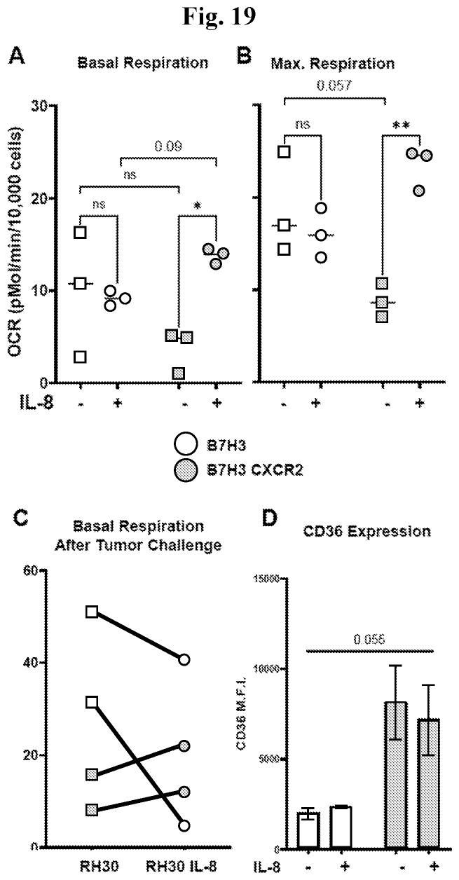

In the absence of IL-8, basal and maximal respiration trended lower in B7H3-CXCR2 compared to B7H3 (FIG. 19 A and B) as measured by mitochondrial stress tests using the Agilent Seahorse assay. Following IL-8 stimulation significant elevations in mitochondrial respiration were observed in B7H3-CXCR2 CAR T-cells and not in the B7H3 CAR T cells (FIG. 19A and B). Given that both glycolytic and OXPHOS pathways were upregulated by IL-8 in B7H3-CXCR2 CAR T cells, the metabolic phenotype after challenge with IL-8 producing RMS cells was ascertained. To do this, a 48-hour co-culture of either B7H3 or B7H3-CXCR2 CAR T cells was performed with either RH30 cells or an RH-30 IL-8 overexpressing cell line (n=2). Following the co-culture, the CAR T cells were isolated for metabolic flux analysis. While basal respiration declined in B7H3 CAR T cells during challenge, B7H3-CXCR2 CAR T increased their basal respiration when cultured with IL-8 producing targets (FIG. 19C). Finally, expression of nutrient uptake receptors on B7H3 vs B7H3-CXCR2 CAR T cells was examined and consistently identified an upregulation in the fatty acid scavenging receptor (CD36) (FIG. 19D), which was independent of IL-8 stimulation.

Expression of CD71 (Transferrin receptor) and CD98 (Amino acid transporter), which are highly expressed on activated T-cells, were also analyzed, but these were similar between B7H3 and B7H3 CXCR2 (data not shown). Collectively this data implies that B7H3-CXCR2 CAR T-cells have superior metabolic potential compared to B7H3 alone. In part, this is due to upregulation of the glycolytic and OXPHOS bioenergetic pathways in B7H3-CXCR2 CAR T cells. CXCR2 expression resulted in an intrinsic increased in mitochondrial mass and lipid uptake capacity (FIGS. 18D and 19D), seemingly independent of exogenous IL-8 stimulation. This finding implicates a potential role for lipid metabolism in fueling B7H3-CXCR2 CAR-T cells, which can suggest a role survival and persistence in the TME, where hypoxia, glucose and glutamine depletion disrupts CAR-T cell functioning.

### Example 15

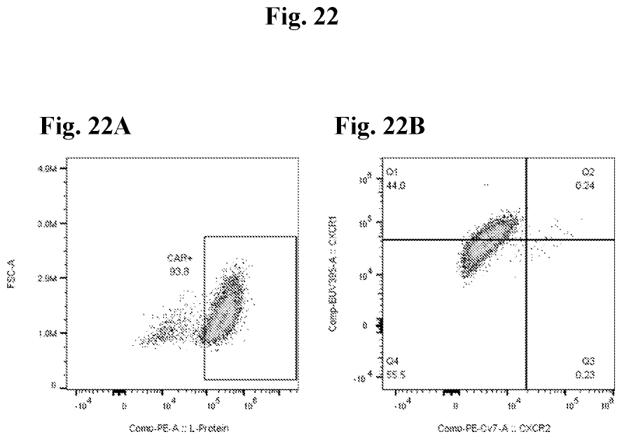

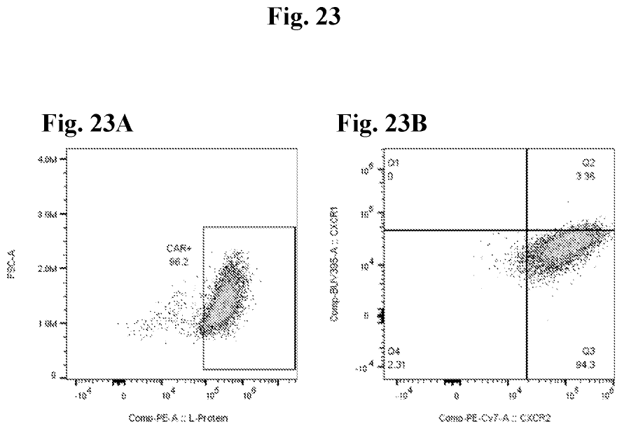

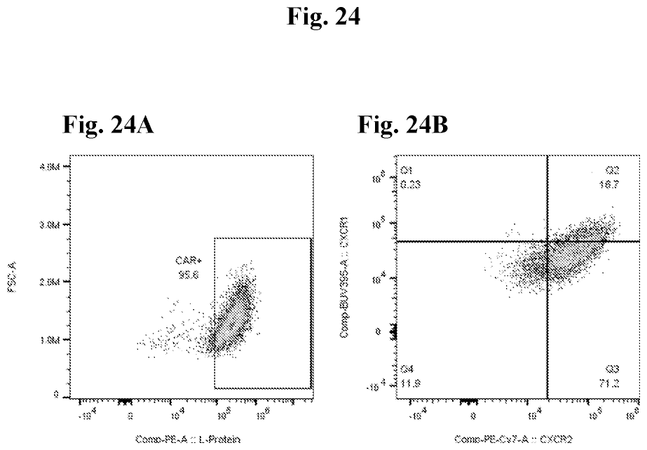

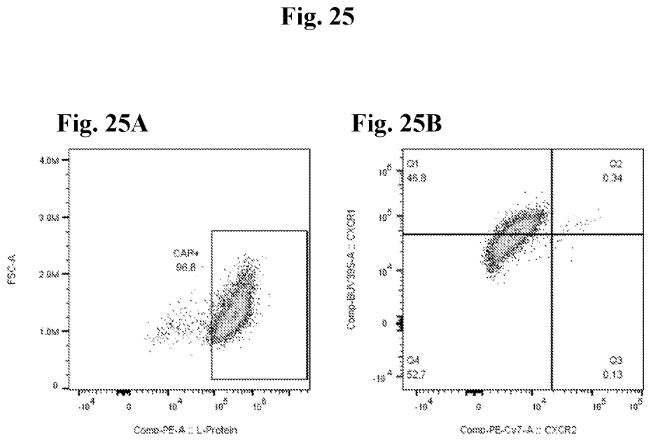

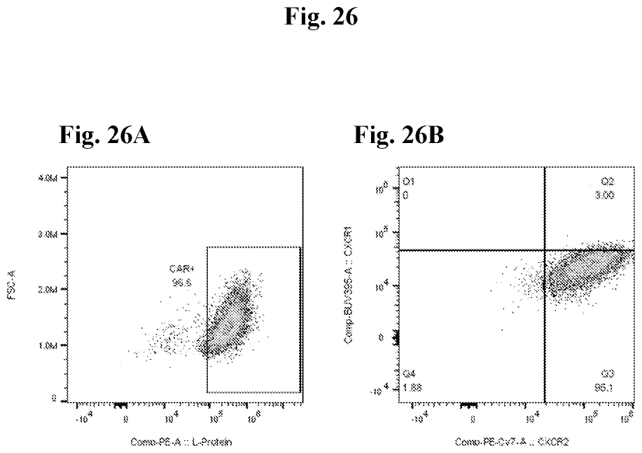

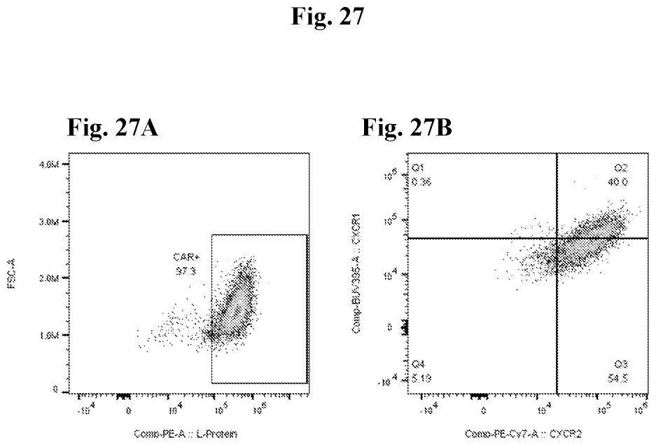

In another exemplary method, expression of B7H3 CAR and one or more chemokine receptors (CXCR1 and/or CXCR2) was measured by flow cytometry. FIGS. 22 and 25 illustrate expression of both B7H3 CAR and CXCR1 in T cells. FIGS. 23 and 26 illustrate expression of both B7H3 CAR and CXCR2 in T cells. FIGS. 24 and 27 illustrate expression of B7H3 CAR, CXCR1, and CXCR2 in T cells.

In brief, retrovirus was made for two different vectors (vector 1, FIGS. 22-24; vector 2, FIGS. 25-27). B7H3 CXCR1, B7H3 CXCR2, B7H3 CXCR1/2 following Verneris lab transfection protocols. T cells were isolated from peripheral blood mononuclear cells (obtained from healthy donor blood) using Ficoll separation and magnetic isolation via StemCell T cell isolation kits. After isolation, T cells were stimulated with 3:1 anti-CD3/28 beads and put into culture in AIMV media with 100 u/ml IL-2. After 2 days in culture, T cells were transduced with the different retroviruses using techniques developed in the Verneris lab. On day 4 of culture, anti-CD3/28 beads were removed using magnets. Around day 5-6 of culture, 500,000 cells were stained for flow cytometry following standard procedures and ran on a Cytek Aurora flow cytometer.

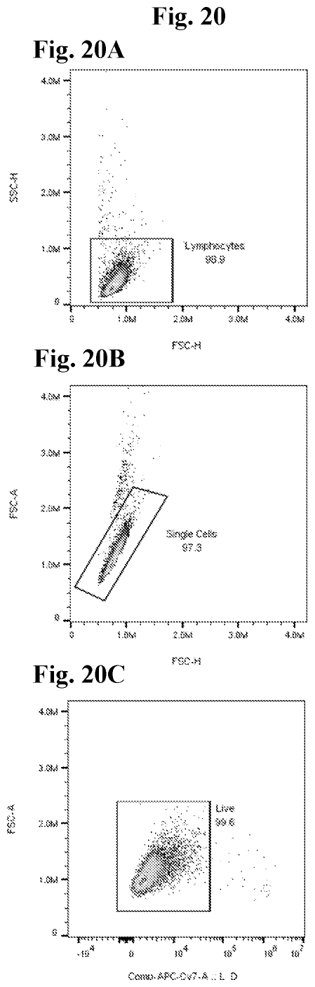

FIG. 20 illustrates the gating strategy for (A) lymphocytes, (B) single cells, and (C) live cells.

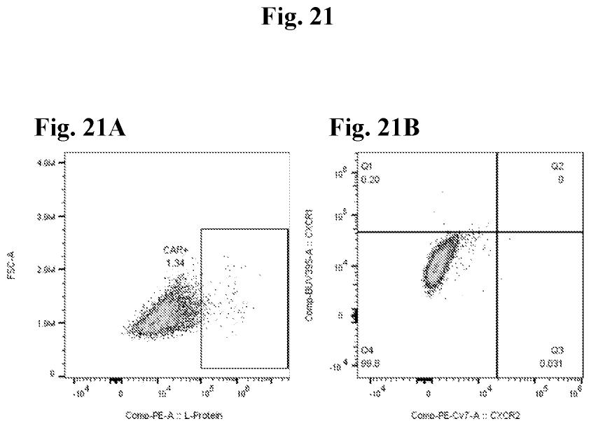

FIG. 21 illustrates flow cytometry data for non-transduced T-cells for (A) L-protein for B7H3 CAR and (B) CXCR1 (y-axis) and CXCR2 (x-axis). 98.66% of non-transduced cells show no discernable L-protein staining (A), where the 1.34% of cells showing high fluorescence represent non-specific binding of the dye to the cells, and 99.8% of cells show no discernable CXCR signal in either the CXCR1 or CXCR2 channels.

FIG. 22 illustrates flow cytometry data for T-cells transduced with vector 1 comprising a B7H3 CAR and CXCR1 chemokine receptor for (A) L-protein for B7H3 CAR and (B) CXCR1 (y-axis) and CXCR2 (x-axis). 93.8% of transduced cells show B7H3 expression (A), and the T-cells show increased CXCR1 fluorescence (B) as compared to the non-transduced control (FIG. 21B).

FIG. 23 illustrates flow cytometry data for T-cells transduced with vector 1 comprising a B7H3 CAR and CXCR2 chemokine receptor for (A) L-protein for B7H3 CAR and (B) CXCR1 (y-axis) and CXCR2 (x-axis). 96.2% of transduced cells show B7H3 expression (A), and the T-cells show increased CXCR2 fluorescence (B) as compared to the non-transduced control (FIG. 22B).

FIG. 24 illustrates flow cytometry data for T-cells transduced with vector 1 comprising a B7H3 CAR, CXCR1, and CXCR2 chemokine receptor for (A) L-protein for B7H3 CAR and (B) CXCR1 (y-axis) and CXCR2 (x-axis). 95.6% of transduced cells show B7H3 expression (A), and the T-cells show increased CXCR1 and CXCR2 fluorescence (B) as compared to the non-transduced control (FIG. 22B).

FIG. 25 illustrates flow cytometry data for T-cells transduced with vector 2 comprising a B7H3 CAR and CXCR1 chemokine receptor for (A) L-protein for B7H3 CAR and (B) CXCR1 (y-axis) and CXCR2 (x-axis). 96.8% of transduced cells show B7H3 expression (A), and the T-cells show increased CXCR1 fluorescence (B) as compared to the non-transduced control (FIG. 21B).

FIG. 23 illustrates flow cytometry data for T-cells transduced with vector 2 comprising a B7H3 CAR and CXCR2 chemokine receptor for (A) L-protein for B7H3 CAR and (B) CXCR1 (y-axis) and CXCR2 (x-axis). 96.6% of transduced cells show B7H3 expression (A), and the T-cells show increased CXCR2 fluorescence (B) as compared to the non-transduced control (FIG. 22B).

FIG. 24 illustrates flow cytometry data for T-cells transduced with vector 2 comprising a B7H3 CAR, CXCR1, and CXCR2 chemokine receptor for (A) L-protein for B7H3 CAR and (B) CXCR1 (y-axis) and CXCR2 (x-axis). 97.3% of transduced cells show B7H3 expression (A), and the T-cells show increased CXCR1 and CXCR2 fluorescence (B) as compared to the non-transduced control (FIG. 22B).

### Example 16

In another exemplary method, chemotaxis of B7H3 CAR T cells comprising one or more chemokine receptors (CXCR1 and/or CXCR2) as compared to a non-transduced T cell control was measured using microscopy in the presence of 100 ng/mL IL-8.

Briefly, a 96 well Incucyte chemotaxis plate was prepared by coating the inserts with 20 uL of 20 ug/mL Protein G and placed at 37 degrees for 1 hour. Next, inserts were washed with PBS by pipetting 40 uL of PBS/insert and removing 60 uL. ICAM 20 uL/well of 5 ug/mL was then added to each insert and incubated for 2 hours at 37 degrees. Both sides of the membrane were blocked with PBS+1% BSA for 30 minutes at room temperature and then washed with PBS. PBS was removed. 2500 of each cell type (B7H3-CXCR1, B7H3-CXCR2, B7H3-CXCR1/2 or non-transduced T cells) were added above the membrane in 60 uL of RPMI+0.5% fetal bovine serum (FBS) and done in triplicate. To the bottom well, either 200 uL of media only (RPMI+0.5% FBS) or 200 uL of media+100 ng/mL recombinant IL-8. Each condition was done in triplicate. Plate was run on the Incucyte for 48 hours.

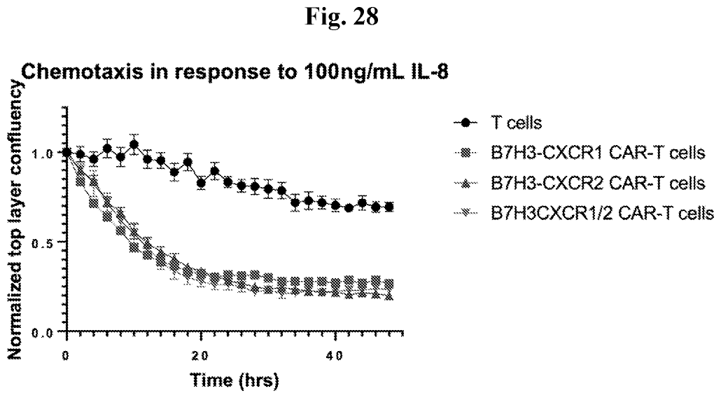

Results show a similar decrease in T cells in the top well in all the CAR T cell types as compared to the non-transduced T cells, representing migration to the bottom well towards the IL-8 gradient (FIG. 28). Each cell type was normalized to its media only condition.

All the COMPOSITIONS and METHODS disclosed and claimed herein may be made and executed without undue experimentation in light of the present disclosure. While the COMPOSITIONS and METHODS have been described in terms of preferred embodiments, it will be apparent to those of skill in the art that variation may be applied to the COMPOSITIONS and METHODS and in the steps or in the sequence of steps of the METHODS described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

What is claimed is:

1. A polynucleotide construct encoding at least one chimeric antigen receptor (CAR) and at least one chemokine receptor, wherein the CAR comprises a single-chain variable fragment (scFv) able to bind to B7H3 (B7 Homolog 3, CD276).

2. The polynucleotide construct according to claim 1, wherein the CAR polynucleotide comprises at least 85% identity up to 100% identity to the polynucleotide represented by SEQ ID NO: 1.

3. The polynucleotide construct according to any one of claim 1 or 2, wherein the polynucleotide encoding the chemokine receptor comprises a polynucleotide encoding at least one of C-X-C chemokine receptor 1 (CXCR1), C-X-C chemokine receptor 2 (CXCR2), C-X-C chemokine receptor 3 (CXCR3), C-X-C chemokine receptor 4 (CXCR4), C-X-C chemokine receptor 5 (CXCR5), C-X-C chemokine receptor 6 (CXCR6), C-C chemokine receptor 1 (CCR1), C-C chemokine receptor 2 (CCR2), C-C chemokine receptor 3 (CCR3), C-C chemokine receptor 4 (CCR4), C-C chemokine receptor 5 (CCR5), C-C chemokine receptor 6 (CCR6), C-C chemokine receptor 7 (CCR7), C-C chemokine receptor 8 (CCR8), C-C chemokine receptor 9 (CCR9), C-C chemokine receptor 10 (CCR10), C-C chemokine receptor 11 (CCR11), C chemokine receptor (XCR1), CX₃C chemokine receptor (CX3CR1), midkine receptor, or a combination thereof or biologically active fragment thereof.

4. The polynucleotide construct according to any one of claim 1 or 2, wherein the polynucleotide encoding the chemokine receptor comprises a polynucleotide comprising CXCR2 or a biologically active fragment thereof.

5. The polynucleotide construct according to claim 4, wherein the polynucleotide encoding CXCR2 comprises at least 85% identity up to 100% identity to the polynucleotide represented by SEQ ID NO: 2.

6. The polynucleotide construct according to any one of claims 1 through 5, wherein the polynucleotide construct encodes a fusion protein comprising a CAR and a chemokine receptor linked via a polypeptide linker.

7. The polynucleotide construct according to claim 6, wherein the polypeptide linker comprises a self-cleaving polypeptide.

8. The polynucleotide construct according to claim 7, wherein the self-cleaving polypeptide is a 2A peptide.

9. A cell comprising a polynucleotide construct according to any one of claims 1 through 8.

10. One or more polypeptides encoded by a polynucleotide construct according to any one of claims 1 through 8.

11. A cell comprising one or more polypeptides of claim 10.

12. A vector comprising a polynucleotide according to any one of claims 1 through 8.

13. The vector according to claim 12, wherein the vector is a viral vector, or baboon pseudotyped vector.

14. The vector according to any one of claims 12 through 13, wherein the vector is a viral vector and the viral vector is a lentiviral, an adenoviral adeno-associated viral, a herpesviral, or a retroviral vector.

15. A cell comprising the vector according to any one of claims 12 through 14.

16. A composition comprising:
a. a first polypeptide comprising a CAR; and
b. a second polypeptide comprising a chemokine receptor.

17. The composition of claim 16, wherein the first and second polypeptides are linked by a polypeptide linker.

18. The composition of claim 17, wherein the polypeptide peptide linker comprises a cleavage site.

19. The composition of claim 18, wherein the cleavage site comprises a self-cleaving polypeptide.

20. The composition of claim 19, wherein the self-cleaving polypeptide comprises a 2A peptide.

21. The composition of any one of claims 16 through 20, wherein the first and the second polypeptides are separate polypeptides.

22. The composition of any one of claims 16 through 21, wherein the first polypeptide comprising a CAR comprises a single-chain variable fragment (scFv) able to bind to B7H3 (B7 Homolog 3, CD276).

23. The composition of claim 22, wherein the first polypeptide comprising a CAR comprises a sequence at least 85% identical up to 100% identical to a polypeptide encoded by SEQ ID NO: 1.

24. The composition of any one of claims 16 through 23, wherein the second polypeptide comprising a chemokine receptor comprises a Cys-Cys motif receptor (CCR) or a Cys-X-Cys motif receptor (CXCR).

25. The composition of claim 24, wherein the second polypeptide comprising a chemokine receptor comprises a C-X-C chemokine receptor 1 (CXCR1), C-X-C chemokine receptor 2 (CXCR2), C-X-C chemokine receptor 3 (CXCR3), C-X-C chemokine receptor 4 (CXCR4), C-X-C chemokine receptor 5 (CXCR5), C-X-C chemokine receptor 6 (CXCR6), C-C chemokine receptor 1 (CCR1), C-C chemokine receptor 2 (CCR2), C-C chemokine receptor 3 (CCR3), C-C chemokine receptor 4 (CCR4), C-C chemokine receptor 5 (CCR5), C-C chemokine receptor 6 (CCR6), C-C chemokine receptor 7 (CCR7), C-C chemokine receptor 8 (CCR8), C-C chemokine receptor 9 (CCR9), C-C chemokine receptor 10 (CCR10), C-C chemokine receptor 11 (CCR11), C chemokine receptor (XCR1), CX₃C chemokine receptor (CX3CR1), midkine receptor, or a combination thereof or biologically active fragment thereof.

26. The composition of claim 25, wherein the second polypeptide comprising a chemokine receptor comprises CXCR1 or a CXCR2.

27. The composition of claim 26, wherein the second polypeptide comprising a chemokine receptor comprises CXCR1.

28. The composition of claim 27, wherein the second polypeptide comprising a chemokine receptor is at least 85% up to 100% identical to a polypeptide encoded by SEQ ID NO: 11.

29. The composition of claim 26, wherein the second polypeptide comprising a chemokine receptor comprises CXCR2.

30. The composition of claim 29, wherein the second polypeptide comprising a chemokine receptor is at least 85% up to 100% identical to a polypeptide encoded by SEQ ID NO: 2.

31. One or more polynucleotides encoding a composition of any one of claims 16 through 30.

32. A cell comprising one or more polypeptides of anyone of claims 16 through 30 and/or one or more polynucleotide of claim 31.

33. The cell according to any one of claims 9, 11, 15, or 32, wherein the cell comprises a human immune cell or a human stem cell.

34. The cell according to claim 33, wherein the human immune cell comprises a neutrophil, eosinophil, basophil, mast cell, monocyte, macrophage, dendritic cell, natural killer cell, or a lymphocyte.

35. The cell according to claim 34, wherein the human immune cell comprises a natural killer (NK) cell.

36. The cell according to claim 34, wherein the human immune cell comprises a T cell.

37. The cell according to claim 36, wherein the T cell comprises a CD8+ T cell, a CD4+ T cell, a CD8+/CD4+ double positive T cell, or a combination thereof.

38. The cell according to claim 37, wherein the CD8+ T cell comprises a naive CD8+ T cell, a central memory CD8+ T cell, an effector memory CD8+ T cell, a bulk CD8+ T cell, or a combination thereof.

39. The cell according to claim 37, wherein the CD4+ T cell comprises a naive CD4+ T cell, a central memory CD4+ T cell, an effector memory CD4+ T cell, a bulk CD4+ T cell, or a combination thereof.

40. The cell according to claim 36, wherein the T cell comprises a precursor T cell.

41. The cell according to claim 33, wherein the human stem cell comprises a human pluripotent, multipotent stem cell, embryonic stem cell, induced pluripotent stem cell, hematopoietic stem cell, CD34+ cell.

42. The cell according to claim 41, wherein the human stem cell comprises a hematopoietic stem cell.

43. The cell according to claim 41, wherein the human stem cell comprises an induced pluripotent stem cell.

44. The cell according to any one of claims 32 through 43, wherein the cell comprises at least 1.2, 1.4, 1.6, 1.8, 2, 4, 5, 6, 7, 8, or 9-fold and/or no more than 10, 9, 8, 7, 6, 5, 4, 2, 1.8, 1.6, or 1.4-fold increased mitochondrial mass as compared to a corresponding cell lacking a chemokine receptor.

45. The cell according to claim 44, wherein the cell comprises at least 2-fold increased mitochondrial mass as compared to a corresponding cell lacking a chemokine receptor.

46. The cell according to any one of claims 32 through 45, wherein the cell comprises at least 1.2, 1.4, 1.6, 1.8, 2, 4, 5, 6, 7, 8, or 9-fold and/or no more than 10, 9, 8, 7, 6, 5, 4, 2, 1.8, 1.6, or 1.4-fold increased ATP production as compared to a corresponding cell lacking a chemokine receptor.

47. The cell according to claim 46, wherein the cell comprises at least 2-fold increased ATP production as compared to a corresponding cell lacking a chemokine receptor.

48. An engineered T cell comprising:
a. a first polynucleotide encoding a first polypeptide comprising a CAR; and
b. a second polynucleotide encoding a second polypeptide comprising a chemokine receptor.

49. The engineered T cell of claim 48, wherein the second polypeptide comprising a chemokine receptor comprises CXCR1 or CXCR2.

50. The engineered T cell of claim 49, wherein the second polypeptide comprising a chemokine receptor comprises CXCR1.

51. The engineered T cell of claim 49, wherein the second polypeptide comprising a chemokine receptor comprises CXCR2.

52. The engineered T cell of any one of claims 48 through 51, further comprising a third polynucleotide encoding a third polypeptide comprising CXCR1 or CXCR2, wherein the third polynucleotide encodes a chemokine receptor different from the chemokine receptor encoded by second polynucleotide.

53. The engineered T cell of any one of claims 48 through 52, wherein the CAR comprises a single-chain variable fragment (scFv) able to bind to B7H3 (B7 Homolog 3, CD276).

54. The engineered T cell of claim 53, wherein the first polynucleotide comprises a sequence at least 85% identical up to 100% identical to SEQ ID NO: 1.

55. The engineered T cell of any one of claims 48 through 54, wherein the second or third polynucleotide is at least 85% up to 100% identical to SEQ ID NO: 11.

56. The engineered T cell of any one of claims 48 through 54, wherein the second or third polynucleotide is at least 85% up to 100% identical to SEQ ID NO: 2.

57. The engineered T cell of any one of claims 48 through 54, wherein the second polynucleotide is at least 85% up to 100% identical to SEQ ID NO: 2 and the third polynucleotide is at least 85% up to 100% identical to SEQ ID NO: 11.

58. The engineered T cell of any one of claims 48 through 57, wherein the engineered T cell comprises a CD8+ T cell, a CD4+ T cell, a CD8+/CD4+ double positive T cell, or a combination thereof.

59. The engineered T cell of claim 58, wherein the CD8+ T cell comprises a naive CD8+ T cell, a central memory CD8+ T cell, an effector memory CD8+ T cell, a bulk CD8+ T cell, or a combination thereof.

60. The engineered T cell of claim 58, wherein the CD4+ T cell comprises a naive CD4+ T cell, a central memory CD4+ T cell, an effector memory CD4+ T cell, a bulk CD4+ T cell, or a combination thereof.

61. The engineered T cell of any one of claims 48 through 57, wherein the engineered T cell comprises a precursor T cell.

62. A pharmaceutical composition comprising a cell of any one of claims 9, 11, 15, or 32 through 61 and a pharmaceutically acceptable excipient or carrier.

63. A method of generating a cell population expressing a polypeptide according to claim 10 comprising:
a. introducing the vector according to any one of claims 12 through 14 or a transposon including a polynucleotide according to any one of claims 1 to 5 into a lymphocyte population or editing a lymphocyte population to express a polypeptide according to claim 10;
b. culturing the lymphocyte population in media and one or more of an anti-CD3 antibody, an anti-CD28 antibody, a cytokine; and
c. enriching for the lymphocyte population expressing the polypeptide according to claim 10.

64. The method according to claim 63, wherein enriching for the lymphocyte population comprises contacting the lymphocyte population comprising the vector according to any one of claims 6-8 with a selection reagent.

65. The method according to claim 64, wherein the selection reagent comprises methotrexate or other suitable selection agent.

66. The method according to any one of claims 63 through 65, wherein the cytokine comprises one or more of interleukin-2 (IL-2), interleukin-7 (IL-17), interleukin-15 (IL-15), interleukin-7 (IL-7), interleukin-12 (IL-12), interleukin-18 (IL-18), interleukin-21 (IL-21), or a combination thereof.

67. The method according to any one of claims 63 through 66, wherein the lymphocyte population comprises comprise CD8+ T cell, a CD4+ T cell, a CD8+/CD4+ double positive T cell, or a combination thereof.

68. The method according to claim 63, wherein the lymphocyte population is further exposed to interleukin-8 (IL-8) to induce caspase production in the lymphocyte population.

69. A method of killing a target cell comprising surface expressed B7H3, the method comprising contacting the target cell with a cell of any one of claims 9, 11, 15, or 32 through 61 or a pharmaceutical composition of claim 62.

70. The method of claim 69, wherein the target cell secretes a cytokine.

71. The method of claim 70, wherein the cytokine comprises a chemokine.

72. The method of claim 71, wherein the chemokine comprises an interleukin.

73. The method of claim 72, wherein the interleukin comprises IL-1, 2, 3, 5, 6, 7, 8, or a combination thereof.

74. The method of claim 73, wherein the interleukin comprises IL-6, IL-8, or a combination thereof.

75. The method of claim 74, wherein the interleukin comprises IL-8.

76. A method of preventing development of, treating, or ameliorating a cancer in a subject, the method comprising administering the pharmaceutical composition of claim 62.

77. The method according to claim 76, wherein the cancer comprises a tumor or a malignancy.

78. The method according to any one of claim 76 or 77, further comprising irradiating the tumor prior to, simultaneously, or after administering the composition to the subject.

79. The method according to claim 78, wherein irradiating the tumor comprises administering at least one dose of radiation ranging from about 2 Gy to about 50 Gy.

80. The method according to any one of claims 76 through 79, wherein the tumor comprises at least one B7H3⁺ cell, at least one IL-8+ cell, or a combination thereof.

81. The method according to any one of claims 76 through 80, wherein the administration is intravenously, by bolus, intrarenally, topically or other suitable mode.

82. The method according to any one of claims 76 through 80, wherein the administration is locally to the tumor in the subject.

83. The method according to any one of claims 76 through 82, wherein the tumor comprises a solid tumor.

84. The method according to any one of claims 76 through 82, wherein the tumor comprises breast, lung, brain, head and neck, prostate, esophageal, stomach or other gastrointestinal tumor, colon, liver, kidney, eye, skin, or other tumor or blood malignancy or other solid tumor or other malignancy.

85. The method according to any one of claims 76 through 83, wherein the tumor comprises a sarcoma.

86. The method according to claim 85, wherein the sarcoma comprises a bone sarcoma, a soft-tissue sarcoma, or other sarcoma or a combination thereof.

87. The method according to claim 86, wherein the bone sarcoma comprises one or more of osteosarcoma, chondrosarcoma, poorly differentiated round/spindle cell tumors, Ewing sarcoma, hemangioendothelioma, angiosarcoma, fibrosarcoma/myofibrosarcoma, chordoma, adamantinoma, liposarcoma, leiomyosarcoma, malignant peripheral nerve sheath tumor, rhabdomyosarcoma, synovial sarcoma, malignant solitary fibrous tumor, or other bone sarcoma.

88. The method according to claim 86, wherein the soft-tissue sarcoma comprises one or more of liposarcoma, atypical lipomatous tumor, dermatofibrosarcoma protuberans, malignant solitary fibrous tumor, inflammatory myofibroblastic tumor, low-grade myofibroblastic sarcoma, fibrosarcoma, myxofibrosarcoma, low-grade fibromyxoid sarcoma, giant cell tumor of soft tissues, leiomyosarcoma, malignant glomus tumor, rhabdomyosarcoma, hemangioendothelioma, angiosarcoma of soft tissue, extraskeletal osteosarcoma, gastrointestinal stromal tumor, malignant, malignant peripheral nerve sheath tumor, malignant Triton tumor, malignant granular cell tumor, malignant ossifying fibromyxoid tumor, stromal sarcoma not otherwise specified, myoepithelial carcinoma, malignant phosphaturic mesenchymal tumor, synovial sarcoma, epithelioid sarcoma, alveolar soft part sarcoma, clear cell sarcoma of soft tissue, extraskeletal myxoid chondrosarcoma, extraskeletal Ewing sarcoma, desmoplastic small round cell tumor, extrarenal rhabdoid tumor, perivascular epithelioid cell tumor, intimal sarcoma, undifferentiated spindle cell sarcoma, undifferentiated pleomorphic sarcoma, undifferentiated round cell sarcoma, undifferentiated epithelioid sarcoma, undifferentiated sarcoma, not otherwise specified, or other soft tissue sarcoma or a combination thereof.


SEQUENCE LISTING (17 sequences)

SEQ ID NO: 1 - DNA, 1711 bp, Artificial Sequence
  Description of Artificial Sequence Synthetic polynucleotide
gcccgccaccatgctgctgctggtgacatccctgctgctgtgcgaactgcctcatcccgctttcctgctgattcctgaagtccagctggtcgagagcggaggaggactggtgcagcctggaggatcactgagactgagctgcgccgcttccggattcacctttagctccttcggcatgcactgggtgaggcaggcaccaggaaaaggcctggagtgggtcgcttacatctctagtgactcaagcgccatctactatgcagataccgtgaaaggcaggtttacaatcagtcgcgacaacgctaagaattccctgtatctgcagatgaactctctgcgcgacgaggatacagcagtctactattgcgggcggggaagagaaaatatctactatggaagccgactggactactggggacagggaaccacagtgacagtctcctctggaggaggaggaagcggaggaggaggatccggaggaggcgggtctgatatccagctgactcagagcccctccttcctgtctgccagtgtgggcgacagggtcactattacctgtaaggcatcccagaacgtggataccaatgtcgcctggtaccagcagaagcccgggaaagcacctaaggccctgatctattcagccagctaccgatattctggcgtgccaagtcggttctccggatctggcagtgggactgactttacactgactattagttcactgcagcccgaagattttgctacctactattgtcagcagtacaataactacccattcaccttcggacaggggacaaaactggaaatcaaagaaagcaagtacggaccgccctgccccccttgccctggccagcctagagaaccccaggtgtacaccctgcctcccagccaggaagagatgaccaagaaccaggtgtccctgacctgcctggtcaaaggcttctaccccagcgatatcgccgtggaatgggagagcaacggccagcccgagaacaactacaagaccaccccccctgtgctggacagcgacggcagcttcttcctgtactcccggctgaccgtggacaagagccggtggcaggaaggcaacgtcttcagctgcagcgtgatgcacgaggccctgcacaaccactacacccagaagtccctgagcctgagcctgggcaagatgttctgggtgctggtggtggtcggaggcgtgctggcctgctacagcctgctggtcaccgtggccttcatcatcttttgggtgaaacggggcagaaagaaactcctgtatatattcaaacaaccatttatgagaccagtacaaactactcaagaggaagatggctgtagctgccgatttccagaagaagaagaaggaggatgtgaactgcgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaagg

SEQ ID NO: 2 - DNA, 1080 bp, Artificial Sequence
  Description of Artificial Sequence Synthetic polynucleotide
atggaagattttaacatggagagtgacagctttgaagatttctggaaaggtgaagatcttagtaattacagttacagctctaccctgcccccttttctactagatgccgccccatgtgaaccagaatccctggaaatcaacaagtattttgtggtcattatctatgccctggtattcctgctgagcctgctgggaaactccctcgtgatgctggtcatcttatacagcagggtcggccgctccgtcactgatgtctacctgctgaacctagccttggccgacctactctttgccctgaccttgcccatctgggccgcctccaaggtgaatggctggatttttggcacattcctgtgcaaggtggtctcactcctgaaggaagtcaacttctatagtggcatcctgctactggcctgcatcagtgtggaccgttacctggccattgtccatgccacacgcacactgacccagaagcgctacttggtcaaattcatatgtctcagcatctggggtctgtccttgctcctggccctgcctgtcttacttttccgaaggaccgtctactcatccaatgttagcccagcctgctatgaggacatgggcaacaatacagcaaactggcggatgctgttacggatcctgccccagtcctttggcttcatcgtgccactgctgatcatgctgttctgctacggattcaccctgcgtacgctgtttaaggcccacatggggcagaagcaccgggccatgcgggtcatctttgctgtcgtcctcatcttcctgctctgctggctgccctacaacctggtcctgctggcagacaccctcatgaggacccaggtgatccaggagacctgtgagcgccgcaatcacatcgaccgggctctggatgccaccgagattctgggcatccttcacagctgcctcaaccccctcatctacgccttcattggccagaagtttcgccatggactcctcaagattctagctatacatggcttgatcagcaaggactccctgcccaaagacagcaggccttcctttgttggctcttcttcagggcacacttccactactctc

SEQ ID NO: 3 - protein, 18 aa, Thosea asigna virus
EGRGSLLTCGDVEENPGP

SEQ ID NO: 4 - protein, 19 aa, Teschovirus A
ATNFSLLKQAGDVEENPGP

SEQ ID NO: 5 - protein, 20 aa, Equine rhinitis A virus
QCTNYALLKLAGDVESNPGP

SEQ ID NO: 6 - protein, 22 aa, Foot-and-mouth disease virus
VKQTLNFDLLKLAGDVESNPGP

SEQ ID NO: 7 - DNA, 38 bp, Artificial Sequence
  Description of Artificial Sequence Synthetic primer
ccctctccactgccgctagcccttgggggcagggcctg

SEQ ID NO: 8 - DNA, 60 bp, Artificial Sequence
  Description of Artificial Sequence Synthetic primer
ctgccgaccccgggggtggaccatcctctagactgctcgaggcccgccaccatgctgctg

SEQ ID NO: 9 - DNA, 1217 bp, Artificial Sequence
  Description of Artificial Sequence Synthetic polynucleotide
tgcaggccctgcccccaagggctagcggcagtggagagggcagaggaagtctgctaacatgcggtgacgtcgaggagaatcctggcccaatggaagattttaacatggagagtgacagctttgaagatttctggaaaggtgaagatcttagtaattacagttacagctctaccctgcccccttttctactagatgccgccccatgtgaaccagaatccctggaaatcaacaagtattttgtggtcattatctatgccctggtattcctgctgagcctgctgggaaactccctcgtgatgctggtcatcttatacagcagggtcggccgctccgtcactgatgtctacctgctgaacctagccttggccgacctactctttgccctgaccttgcccatctgggccgcctccaaggtgaatggctggatttttggcacattcctgtgcaaggtggtctcactcctgaaggaagtcaacttctatagtggcatcctgctactggcctgcatcagtgtggaccgttacctggccattgtccatgccacacgcacactgacccagaagcgctacttggtcaaattcatatgtctcagcatctggggtctgtccttgctcctggccctgcctgtcttacttttccgaaggaccgtctactcatccaatgttagcccagcctgctatgaggacatgggcaacaatacagcaaactggcggatgctgttacggatcctgccccagtcctttggcttcatcgtgccactgctgatcatgctgttctgctacggattcaccctgcgtacgctgtttaaggcccacatggggcagaagcaccgggccatgcgggtcatctttgctgtcgtcctcatcttcctgctctgctggctgccctacaacctggtcctgctggcagacaccctcatgaggacccaggtgatccaggagacctgtgagcgccgcaatcacatcgaccgggctctggatgccaccgagattctgggcatccttcacagctgcctcaaccccctcatctacgccttcattggccagaagtttcgccatggactcctcaagattctagctatacatggcttgatcagcaaggactccctgcccaaagacagcaggccttcctttgttggctcttcttcagggcacacttccactactctctaaggatccgataaaataaaagattttatttagtctccagaaaaaggg

SEQ ID NO: 10 - protein, 350 aa, Artificial Sequence
  Description of Artificial Sequence Synthetic polypeptide
MSNITDPQMWDFDDLNFTGMPPADEDYSPCMLETETLNKYVVIIAYALVFLLSLLGNSLVMLVILYSRVGRSVTDVYLLNLALADLLFALTLPIWAASKVNGWIFGTFLCKVVSLLKEVNFYSGILLLACISVDRYLAIVHATRTLTQKRHLVKFVCLGCWGLSMNLSLPFFLFRQAYHPNNSSPVCYEVLGNDTAKWRMVLRILPHTFGFIVPLFVMLFCYGFTLRTLFKAHMGQKHRAMRVIFAVVLIFLLCWLPYNLVLLADTLMRTQVIQESCERRNNIGRALDATEILGFLHSCLNPIIYAFIGQNFRHGFLKILAMHGLVSKEFLARHRVTSYTSSSVNVSSNL

SEQ ID NO: 11 - DNA, 1053 bp, Artificial Sequence
  Description of Artificial Sequence Synthetic polynucleotide
atgtcaaatattacagatccacagatgtgggattttgatgatctaaatttcactggcatgccacctgcagatgaagattacagcccctgtatgctagaaactgagacactcaacaagtatgttgtgatcatcgcctatgccctagtgttcctgctgagcctgctgggaaactccctggtgatgctggtcatcttatacagcagggtcggccgctccgtcactgatgtctacctgctgaacctggccttggccgacctactctttgccctgaccttgcccatctgggccgcctccaaggtgaatggctggatttttggcacattcctgtgcaaggtggtctcactcctgaaggaagtcaacttctacagtggcatcctgctgttggcctgcatcagtgtggaccgttacctggccattgtccatgccacacgcacactgacccagaagcgtcacttggtcaagtttgtttgtcttggctgctggggactgtctatgaatctgtccctgcccttcttccttttccgccaggcttaccatccaaacaattccagtccagtttgctatgaggtcctgggaaatgacacagcaaaatggcggatggtgttgcggatcctgcctcacacctttggcttcatcgtgccgctgtttgtcatgctgttctgctatggattcaccctgcgtacactgtttaaggcccacatggggcagaagcaccgagccatgagggtcatctttgctgtcgtcctcatcttcctgctttgctggctgccctacaacctggtcctgctggcagacaccctcatgaggacccaggtgatccaggagasctgtgagcgccgcaacaacatcggccgggccctggatgccactgagattctgggatttctccatagctgcctcaaccccatcatctacgccttcatcggccaaaattttcgccatggattcctcaagatcctggctatgcatggcctggtcagcaaggagttcttggcacgtcatcgtgttacctcctacacttcttcgtctgtcaatgtctcttccaacctctga

SEQ ID NO: 12 - protein, 360 aa, Artificial Sequence
  Description of Artificial Sequence Synthetic polypeptide
MEDFNMESDSFEDFWKGEDLSNYSYSSTLPPFLLDAAPCEPESLEINKYFVVIIYALVFLLSLLGNSLVMLVILYSRVGRSVTDVYLLNLALADLLFALTLPIWAASKVNGWIFGTFLCKVVSLLKEVNFYSGILLLACISVDRYLAIVHATRTLTQKRYLVKFICLSIWGLSLLLALPVLLFRRTVYSSNVSPACYEDMGNNTANWRMLLRILPQSFGFIVPLLIMLFCYGFTLRTLFKAHMGQKHRAMRVIFAVVLIFLLCWLPYNLVLLADTLMRTQVIQETCERRNHIDRALDATEILGILHSCLNPLIYAFIGQKFRHGLLKILAIHGLISKDSLPKDSRPSFVGSSSGHTSTTL

SEQ ID NO: 13 - DNA, 2459 bp, Artificial Sequence
  Description of Artificial Sequence Synthetic polynucleotide
actctgatctctgactgcagctcctactgttggacacacctggccggtgcttcagttagatcaaaccattgctgaaactgaagaggacatgtcaaatattacagatccacagatgtgggattttgatgatctaaatttcactggcatgccacctgcagatgaagattacagcccctgtatgctagaaactgagacactcaacaagtatgttgtgatcatcgcctatgccctagtgttcctgctgagcctgctgggaaactccctggtgatgctggtcatcttatacagcagggtcggccgctccgtcactgatgtctacctgctgaacctggccttggccgacctactctttgccctgaccttgcccatctgggccgcctccaaggtgaatggctggatttttggcacattcctgtgcaaggtggtctcactcctgaaggaagtcaacttctacagtggcatcctgctgttggcctgcatcagtgtggaccgttacctggccattgtccatgccacacgcacactgacccagaagcgtcacttggtcaagtttgtttgtcttggctgctggggactgtctatgaatctgtccctgcccttcttccttttccgccaggcttaccatccaaacaattccagtccagtttgctatgaggtcctgggaaatgacacagcaaaatggcggatggtgttgcggatcctgcctcacacctttggcttcatcgtgccgctgtttgtcatgctgttctgctatggattcaccctgcgtacactgtttaaggcccacatggggcagaagcaccgagccatgagggtcatctttgctgtcgtcctcatcttcctgctttgctggctgccctacaacctggtcctgctggcagacaccctcatgaggacccaggtgatccaggagagctgtgagcgccgcaacaacatcggccgggccctggatgccactgagattctgggatttctccatagctgcctcaaccccatcatctacgccttcatcggccaaaattttcgccatggattcctcaagatcctggctatgcatggcctggtcagcaaggagttcttggcacgtcatcgtgttacctcctacacttcttcgtctgtcaatgtctcttccaacctctgaaaaccatcgatgaaggaatatctcttctcagaaggaaagaataaccaacaccctgaggttgtgtgtggaaggtgatctggctctggacaggcactatctgggttttggggggacgctataggatgtggggaagttaggaactggtgtcttcaggggccacaccaaccttctgaggagctgttgaggtacctccaaggaccggcctttgcacctccatggaaacgaagcaccatcattcccgttgaacgtcacatctttaacccactaactggctaattagcatggccacatctgagccccgaatctgacattagatgagagaacagggctgaagctgtgtcctcatgagggctggatgctctcgttgaccctcacaggagcatctcctcaactctgagtgttaagcgttgagccaccaagctggtggctctgtgtgctctgatccgagctcaggggggtggttttcccatctcaggtgtgttgcagtgtctgctggagacattgaggcaggcactgccaaaacatcaacctgccagctggccttgtgaggagctggaaacacatgttccccttgggggtggtggatgaacaaagagaaagagggtttggaagccagatctatgccacaagaaccccctttacccccatgaccaacatcgcagacacatgtgctggccacctgctgagccccaagtggaacgagacaagcagcccttagcccttcccctctgcagcttccaggctggcgtgcagcatcagcatccctagaaagccatgtgcagccaccagtccattgggcaggcagatgttcctaataaagcttctgttccgtgcttgtccctgtggaagtatcttggttgtgacagagtcaagggtgtgtgcagcattgttggctgttcctgcagtagaatgggggcagcacctcctaagaaggcacctctctgggttgaagggcagtgttccctggggctttaactcctgctagaacagtctcttgaggcacagaaactcctgttcatgcccatacccctggccaaggaagatccctttgtccacaagtaaaaggaaatgctcctccagggagtctcagcttcaccctgaggtgagcatcatcttctgggttaggccttgcctaggcatagccctgcctcaagctatgtgagctcaccagtccctccccaaatgctttccatgagttgcagttttttcctagtctgttttccctccttggagacagggccctgtcggtttattcactgtatgtccttggtgcctggagcctactaaatgctcaataaataatgatcacaggaatgaa

SEQ ID NO: 14 - DNA, 335 bp, Artificial Sequence
  Description of Artificial Sequence Synthetic polynucleotide
caatgttagcccagcctgctatgaggacatgggcaacaatacagcaaactggcggatgctgttacggatcctgccccagtcctttggcttcatcgtgccactgctgatcatgctgttctgctacggattcaccctgcgtacgctgtttaaggcccacatggggcagaagcaccgggccatgcgggtcatctttgctgttgtcctcatcttcctgctctgctggctgccctacaacctggtcctgctggcagacaccctcatgaggacccaggtgatccaggagacctgtgagcgccgcaatcacatcgaccgggctctggatgccaccgagattc

SEQ ID NO: 15 - DNA, 336 bp, Artificial Sequence
  Description of Artificial Sequence Synthetic polynucleotide
ggtcattatctatgccctggtattcctgctgagcctgctgggaaactccctcgtgatgctggtcatcttatacagcagggtcggctgctccgtcactgatgtctacctgctgaacctagccttggccgacctactctttgccctgaccttgcccatctgggccgcctccaaggtgaatggctggatttttggcacattcctgtgcaaggtggtctcactcctgaaggaagtcaacttctatagtggcatcctgctactggcctgcatcagtgtggaccgttacctggccattgtccatgccacacgcacactgacccagaagcgctacttggtcaa

SEQ ID NO: 16 - DNA, 2803 bp, Artificial Sequence
  Description of Artificial Sequence Synthetic polynucleotide
agagacagaaggtggatagacaaatctccaccttcagactggtaggctcctccagaagccatcagacaggaagatgtgaaaatccccagcactcatcccagaatcactaagtggcacctgtcctgggccaaagtcccaggacagacctcattgttcctctgtgggaatacctccccaggagggcatcctggatttcccccttgcaacccaggtcagaagtttcatcgtcaaggttgtttcatcttttttttcctgtctaacagctctgactaccacccaaccttgaggcacagtgaagacatcggtggccactccaataacagcaggtcaggatttaagtttacctcaaaaatggaagattttaacatggagagtgacagctttgaagatttctggaaaggtgaagatcttagtaattacagttacagctctaccctgcccccttttctactagatgccgccccatgtgaaccagaatccctggaaatcaacaagtattttgtggtcattatctatgccctggtattcctgctgagcctgctgggaaactccctcgtgatgctggtcatcttatacagcagggtcggccgctccgtcactgatgtctacctgctgaacctagccttggccgacctactctttgccctgaccttgcccatctgggccgcctccaaggtgaatggctggatttttggcacattcctgtgcaaggtggtctcactcctgaaggaagtcaacttctatagtggcatcctgctactggcctgcatcagtgtggaccgttacctggccattgtccatgccacacgcacactgacccagaagcgctacttggtcaaattcatatgtctcagcatctggggtctgtccttgctcctggccctgcctgtcttacttttccgaaggaccgtctactcatccaatgttagcccagcctgctatgaggacatgggcaacaatacagcaaactggcggatgctgttacggatcctgccccagtcctttggcttcatcgtgccactgctgatcatgctgttctgctacggattcaccctgcgtacgctgtttaaggcccacatggggcagaagcaccgggccatgcgggtcatctttgctgtcgtcctcatcttcctgctctgctggctgccctacaacctggtcctgctggcagacaccctcatgaggacccaggtgatccaggagacctgtgagcgccgcaatcacatcgaccgggctctggatgccaccgagattctgggcatccttcacagctgcctcaaccccctcatctacgccttcattggccagaagtttcgccatggactcctcaagattctagctatacatggcttgatcagcaaggactccctgcccaaagacagcaggccttcctttgttggctcttcttcagggcacacttccactactctctaagacctcctgcctaagtgcagccccgtggggttcctcccttctcttcacagtcacattccaagcctcatgtccactggttcttcttggtctcagtgtcaatgcagcccccattgtggtcacaggaagtagaggaggccacgttcttactagtttcccttgcatggtttagaaagcttgccctggtgcctcaccccttgccataattactatgtcatttgctggagctctgcccatcctgcccctgagcccatggcactctatgttctaagaagtgaaaatctacactccagtgagacagctctgcatactcattaggatggctagtatcaaaagaaagaaaatcaggctggccaacggggtgaaaccctgtctctactaaaaatacaaaaaaaaaaaaaaattagccgggcgtggtggtgagtgcctgtaatcacagctacttgggaggctgagatgggagaatcacttgaacccgggaggcagaggttgcagtgagccgagattgtgcccctgcactccagcctgagcgacagtgagactctgtctcagtccatgaagatgtagaggagaaactggaactctcgagcgttgctgggggggattgtaaaatggtgtgaccactgcagaagacagtatggcagctttcctcaaaacttcagacatagaattaacacatgatcctgcaattccacttataggaattgacccacaagaaatgaaagcagggacttgaacccatatttgtacaccaatattcatagcagcttattcacaagacccaaaaggcagaagcaacccaaatgttcatcaatgaatgaatgaatggctaagcaaaatgtgatatgtacctaacgaagtatccttcagcctgaaagaggaatgaagtactcatacatgttacaacacggacgaaccttgaaaactttatgctaagtgaaataagccagacatcaacagataaatagtttatgattccacctacatgaggtactgagagtgaacaaatttacagagacagaaagcagaacagtgattaccagggactgaggggaggggagcatgggaagtgacggtttaatgggcacagggtttatgtttaggatgttgaaaaagttctgcagataaacagtagtgatagttgtaccgcaatgtgacttaatgccactaaattgacacttaaaaatggtttaaatggtcaattttgttatgtatattttatatcaatttaaaaaaaaacctgagccccaaaaggtattttaatcaccaaggctgattaaaccaaggctagaaccacctgcctatattttttgttaaatgatttcattcaatatcttttttttaataaaccatttttacttgggtgtttata

SEQ ID NO: 17 - protein, 5 aa, Artificial Sequence
  Description of Artificial Sequence Synthetic peptide
GGGGS